SEQUENCE LISTING (2 sequences)

SEQ ID NO: 1 - DNA, 20 bp, synthetic construct
  source 1..20: mol_type = other DNA
agtctgtccaccccctctac

SEQ ID NO: 2 - DNA, 20 bp, synthetic construct
  source 1..20: mol_type = other DNA
actccactccaccgtcaaac

(19) United States
(12) Patent Application Publication
(10) Pub. No.: US 2025/0130239 A1
(43) Pub. Date: Apr. 24, 2025

(54) PROTEIN DEGRADER

(71) Applicant: University of Dundee, Dundee (GB)

(72) Inventors: Alessio Ciulli, Dundee (GB); Adam George Bond, Dundee (GB); Connor George Craigon, Dundee (GB); Kwok-Ho Chan, Stevenage (GB); Andrea Testa, Newhouse, Chapelhall, Motherwell (GB); Dario Alessi, Dundee (GB); Thomas Macartney, Dundee (GB)

(21) Appl. No.: 18/694,524

(22) PCT Filed: Sep. 23, 2022
(86) PCT No.: PCT/GB2022/052408

§ 371 Date: Mar. 22, 2024

(30) Foreign Application Priority Data
Sep. 24, 2021 (GB) 2113656.9

(51) Int. Cl. G01N 33/68; C07D 495/14; C07K 14/47

(52) U.S. Cl. CPC G01N 33/6803; C07D 495/14; C07K 14/47; C07K 2319/70

(57) ABSTRACT

The present invention concerns a method of degrading target proteins, by forming fusion proteins which comprise hole-modified mutant BET bromodomains conjugated to the target protein and using a degrader compound to initiate protein degradation.

### FIELD OF THE INVENTION

The present invention concerns a method of degrading target proteins, by forming fusion proteins which comprise hole-modified mutant BET bromodomains conjugated to the target protein and using a degrader compound to initiate protein degradation. There are also provided compounds of formula (I), which are able to bind to hole-modified mutant BET bromodomains, particularly a mutant Brd4 bromodomain tagged to a target protein. In addition, the present invention concerns degrader compounds of formula (IA), comprising a segment derived from a compound of formula (I) and capable of binding to hole-modified mutant Brd4 bromodomains, a linker, and a segment capable of binding to an E3 ubiquitin ligase. The invention concerns the use of compounds of formula (IA) to degrade proteins, particularly proteins that are endogenously tagged with hole-modified mutant Brd4 bromodomains.

### BACKGROUND

Targeted protein degradation is established as a powerful modality of chemical biology and drug discovery. Proteolysis targeting chimeras (PROTACs) are heterobifunctional molecules, which hijack the ubiquitin proteasome system by recruiting an E3 ubiquitin ligase to a target protein of interest, promoting the protein's polyubiquitination and subsequent proteasomal degradation (Bond, M. J.; Crews, C. M., RSC Chemical Biology 2021, 2 (3), 725-742). The ability to rapidly remove a protein entirely as opposed to merely blocking a single activity or interaction offers an attractive mechanism of action to study the target protein biology, therapeutic potential and to action that pharmacologically. However, the PROTAC approach is limited by the availability of small-molecule ligands that engage the protein target. While good ligands are available for many target proteins, a large proportion of the human proteome lack such binding ligands (Oprea, T. I. et al., Nat Rev Drug Discov. 2018, 17 (5), 317-332). It is therefore important to develop new methodologies to address unligandable proteins, many of which remain unexplored in biology and disease.

To action on proteins that lack binding ligands, a complementary strategy involves modifying the gene which encodes for the protein of interest by adding a tag, also called “degron tag”, which allows small molecules to bind to and directly recruit the E3 ligase to ubiquitinate and degrade the target protein. Examples of tag-based degron systems include: the auxin-inducible degron (AID), whereby a target protein is fused with the AID/IAA17 degron sequence that is recognized by the plant Cullin RING E3 ligase TIR1 in the presence of the molecular glue auxin (Natsume, T.; Kiyomitsu, T.; Saga, Y.; Kanemaki, M. T., Cell Reports 2016, 15 (1), 210-218.), or bumped analogues selectively targeting mutant TIR1 (Yesbolatova, A. et al., Nat Comm. 2020, 11 (1), 5701.); HaloPROTACs—bifunctional molecules that bear a chloroalkane warhead forming a covalent bond with a HaloTag fused to the target protein at one end, and to the E3 ligase von Hippel-Lindau (VHL) at the other end (Tovell, H. et al., ACS Chem Biol. 2019, 14 (5), 882-892); and dTAG, bifunctional molecules which bind to a FKBP12^{F36V} tag that is fused to the target protein at one end, and either cereblon (CRBN) or VHL ligases at the other end (Nabet, B. et al., Nat. Chem. Biol. 2018, 14 (5), 431-441). These approaches have been used successfully to induce targeted protein degradation in cells and in vivo, but they all have disadvantages and limitations. For example, AID methods can be leaky (background target degradation even in prior to auxin dosage), require high concentrations of auxin to work, and also require inconvenient additional engineering to allow for the expression of the non-native plant E3 ligase; all limitations that can lead to possible off-target effects. HaloPROTACs react covalently with the tagged protein so require stoichiometric modification of the tagged protein to induce maximal degradation, and therefore lack the sub-stoichiometric, catalytic mode of action, which is an advantage of non-covalent degraders; as a result HaloPROTACs tend not to achieve complete target degradation and tend to plateau at Dₘₐₓ˜85-90% even at the high doses. CRBN-based dTAGs bear phthalimide-based ligands which can exhibit chemical instability and off-target effects (Ishoey, M. et al., ACS Chem Biol. 2018, 13 (3), 553-560).

Compared with classical inhibition by small molecules, PROTACs offer several potential advantages: (1) PROTACs are expected to exert similar phenotypes to those observed via knockdowns using genetic tools, such as small interfering RNA (siRNA), short hairpin RNA (shRNA), or clustered regularly interspaced short palindromic repeats (CRISPR), because the downstream result is the same in all those cases (i.e., depletion of intracellular protein levels). Elimination of a target protein could give additional effect by disrupting formation of biologically functional complexes. (2) PROTACs can work catalytically (i.e., can be recycled so that one PROTAC molecule can turn over multiple molecules of POI) and so can act “sub-stoichiometrically” (i.e., at fractional occupancy of the target protein). As a result of this, PROTACs often show higher target protein degradation than expected based on their binding affinity to the target protein alone. (3) Target protein degradation by PROTACs can suppress resistant mutation and/or upregulation of POI.

Endogenously tagging proteins with hole-modified mutant bromodomains so that such proteins may be degraded by compounds comprising a segment capable of binding to the hole-modified mutant bromodomains has recently been reported. XY-06-007, a compound comprising a “bump” as part of a segment that binds to Brd4 and a CRBN-based ligand, has been developed by R. P. Nowak et al. in J. Med. Chem., 2021, 64, 15, 11637-11650. XY-06-007 is used to degrade proteins comprising a Brd4^{BD1} L94V tag. The present invention provides an alternative tag-based degron system.

### SUMMARY

As described above, the PROTAC approach for targeted protein degradation is limited to the availability of small-molecule ligands that bind the target protein, and it is important to develop new methodologies to address unligandable proteins. The inventors of the present invention have endogenously tagged proteins with hole-modified mutant Brd4 bromodomains. The resultant proteins may be degraded by degrader compounds comprising a segment capable of binding to hole-modified mutant BET bromodomains, a linker, and a segment capable of binding to an E3 ubiquitin ligase. The degrader compounds bind non-covalently and are selective for the tagged proteins over other proteins that may be present. Such a system is suitable to assess the functional consequences of target protein degradation in genetically engineered models.

In a first teaching, there is provided a method of studying an effect of degrading a target protein within a cell, the method comprising endogenously expressing a fusion protein comprising the target protein, fused to a polypeptide comprising a hole-modified mutant bromodomain;
contacting the fusion protein with a degrader compound comprising a segment capable of binding specifically to the hole-modified mutant bromodomain of the fusion protein, a linker, and a von Hippel-Lindau (VHL) ligand capable of binding to a protein complex comprising elongin B, elongin C and cullin-2, which possesses E3 ubiquitin ligase activity; and observing any effect upon the cell of degrading the target protein.

In one embodiment, the degrader compound is a compound according to formula 1A as defined herein, or embodiments and selected compounds as defined herein.

In one embodiment, the target protein is endogenously tagged (in order to create a fusion protein) with one or more hole-modified mutant bromodomain(s) from the Bromo and Extraterminal domain (BET) proteins, Brd2, Brd3, Brd4 and BrdT and the mutation may occur in one of more bromodomains which are present in the protein, or a bromodomain containing fragment thereof.

In one embodiment, the hole-modified mutant bromodomain is a Brd4_{BD2}L387A, Brd4_{BD2}L387V, Brd4_{BD1}L94A, Brd4_{BD1}L94V, Brd2_{BD2}L383A, Brd2_{BD2}L383V, Brd2_{BD1}L110A, Brd2_{BD1}L110V, Brd3_{BD2}L344A, Brd3_{BD2}L344V, Brd3_{BD1}L70A, Brd3_{BD1}L70V, Brd4_{BD2}L306A, Brd4_{BD2}L306V, Brd4_{BD1}L63A, or Brd4_{BD1}L63V bromodomain. In one embodiment, the hole-modified mutant Brd4 bromodomain is a Brd4_{BD2}L387A or Brd4_{BD2}L387V bromodomain. Numbering according to UniProt.

It will be understood that once bound to E3 ubiquitin ligase, ubiquitin is recruited and bound to the fusion protein, resulting in degradation of the fusion protein by the ubiquitin-proteasome pathway within a cell.

The target protein may be any protein expressed by the cell. Exemplary target proteins include enzymes, structural proteins, hormones, cell surface receptors, tumour associated proteins, etc. The target protein may be a protein that is associated with a disease and/or may be a mutant or wild-type protein. For example, a specific disease may be associated with expression of a mutant protein and the present teaching allows the study of what happens to a cell, when the mutant protein is degraded.

The cell may be any suitable eukaryotic cell. In one embodiment the eukaryotic cell, is a mammalian (e.g. human) cell. The cell may be a normal (non-diseased) or diseased cell, for example. A diseased cell may be a cell which has been derived form a subject with a disease. For example, the subject may be suffering from a cancer, and the cell may be a cancer cell; the subject may be suffering from liver disease and the cell may be a liver cell obtained from the subject; the subject may have kidney disease and the cell may be a kidney cell obtained from the subject, etc. The cell may also be from a cell line previously derived from suitable subjects.

The methods described herein permit the study of any effect of degrading a target protein within the cell. It will be appreciated that a comparison may be made with a corresponding cell, to which a degrader compound has not been added, in order that any effect of degrading the target protein may be observed. Any degraded protein may be quantified by measuring non-degraded or degraded fusion protein in or on the surface of said cells using standard methods for identifying and quantifying proteins.

These methods include, inter alia, using protein specific antibodies linked to a reporter, such as a fluorescent or other reporter, such methods including immunoassay (e.g. ELISA, among others) and immunoblot, absorbance assays, mass spectrometric methods and proteomics methods, among numerous others. Methods for quantifying specific proteins in samples are well known in the art and are readily adapted to methods according to the present disclosure. Assaying for degraded protein and the impact of such degradation on the function of a cell, for example, the growth and/or proliferation of the cell (e.g., cell death) or other characteristic (e.g. biological, physiological) of a cell evidences the importance of the protein of interest to cellular growth and function and establishes whether the target protein is a modulator of a disease state or condition, for example and thus a potential target (bioactive agent, including drugs) for the treatment of said disease state or condition. Identifying a target protein as a pharmaceutical target will allow the development of assays to identify compounds and other bioactive agents exhibiting activity as potential inhibitors and/or agonists of the target protein.

As mentioned above, the target protein is endogenously tagged (fused) with a polypeptide comprising one or more hole-modified mutant bromodomain(s), contained within a BET protein, Brd2, Brd3, Brd4 and BrdT, prior to contact with the degrader compound. In some embodiments, the polypeptide comprising the hole-modified mutant bromodomain is from Brd2, Brd3, Brd4 or BrdT and the mutation is Brd4_{BD2}L387A, Brd4_{BD2}L387V, Brd4_{BD1}L94A, Brd4_{BD1}L94V, Brd2_{BD2}L383A, Brd2_{BD2}L383V, Brd2_{BD1}L110A, Brd2_{BD1}L110V, Brd3_{BD2}L344A, Brd3_{BD2}L344V, Brd3_{BD1}L70A, Brd3_{BD1}L70V, Brd4_{BD2}L306A, Brd4_{BD2}L306V, Brd4_{BD1}L63A, or Brd4_{BD1}L63V.

It is understood that conventional one-amino acid letters are used throughout this disclosure and, for example, L387V means that the Leucine at position 387 of the protein has been replaced by a valine.

The cell may be modified using well known recombinant nucleic acid techniques, such that the endogenous nucleic acid, which encodes the target protein, is modified to express a fusion protein comprising the target protein, fused to (or tagged with) the polypeptide comprising a hole-modified mutant bromodomain. The nucleic acid encoding the target protein may be modified by having a 5′ or 3′ in-frame insertion of a nucleic acid encoding the hole-modified mutant bromodomain. In this manner, the polypeptide comprising a hole-modified mutant bromodomain may, for example, be fused to the N-, or C-terminus of the target protein. Suitable methods of achieving this include homologous recombination (including CRISPR/Cas9, and transposon-mediated system techniques known in the art) and non-homologous end joining techniques known in the art.

Fusion proteins according to the present invention are recombinant fusion proteins, created through engineering of a fusion gene. This typically involves removing the stop codon from the sequence coding for the target protein, then appending the sequence encoding the hole-modified mutant bromodomain protein, or fragment thereof in frame through recombinant techniques as described herein. The introduced hole-modified mutant bromodomain sequence will then be expressed along with the target protein sequence by a cell as a single protein. The fusion protein can be engineered to include the full sequence of both the target and hole-modified bromodomain proteins, or only a portion of one or the other. If the two entities are proteins, spacer peptides may be added which make it more likely that the proteins fold independently and behave as expected.

Thus, in one aspect there is provided a nucleic acid sequence which encodes a fusion protein comprising a target protein, fused to a polypeptide comprising a hole-modified mutant bromodomain as defined herein, the nucleic acid sequence comprising a nucleic acid sequence encoding the target protein and having a 5′- or 3′-in-frame insertion of a nucleic acid encoding the polypeptide comprising a hole-modified mutant bromodomain which, when expressed, results in a fusion protein which is capable of being bound by a degrader compound as described herein.

In one embodiment, the nucleic acid sequence is intended to replace the endogenous nucleic acid sequence which encodes the target protein within a cell.

There is further provided a vector, such as a plasmid or virus vector comprising the nucleic acid sequence of the above aspect.

There is further provided cells (including somatic, embryonic stem cells, induced pluripotent cells) and animals, including in particular non-human animals, the genome of which has been modified to express the nucleic acid sequence of the above aspect. As mentioned above, in some embodiments, the nucleic acid sequence, which encodes the fusion protein of the present teaching, may replace the nucleic acid which encodes the endogenous target protein. In this manner, the target protein may only be expressed within a cell or animal in the form of a fusion protein as described herein.

In addition to developing the fusion proteins as described herein, the inventors have found that compounds of formula (I) are able to selectively bind to hole-modified mutant Brd4 bromodomains within a tagged fusion protein. In addition, degrader compounds of formula (IA), are able to selectively bind and promote degradation of hole-modified mutant Brd4 bromodomains within a tagged protein. Consequently, the degrader compounds of formula (IA), are suitable degraders to assess the functional consequences of target protein degradation in genetically engineered models.

Viewed from another aspect, therefore, the disclosure provides a compound of formula (I) for use in a method of studying an effect of degrading a target protein within a cell (such as the methods described above),

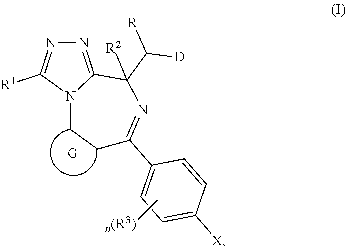

wherein
G is a 5-membered heteroarene optionally substituted with one or two substituents selected from the group consisting of methyl, halo, hydroxy, thiol, halomethyl, amino, methoxy, methylamino, dimethylamino, ethyl, haloethyl, amido, isopropyl and methylthio, or G is a 6-membered arene or heteroarene optionally substituted with methyl, halo, hydroxy and thiol;
R is C₁₋₄alkyl or C₁₋₄haloalkyl;
R¹ is any one selected from the group consisting of C₁₋₄alkyl, C₁₋₄haloalkyl, H and halo;
R² is H, C₁₋₃alkyl, C₁₋₃haloalkyl or halo;
R³ is independently selected from halo, hydroxyl, thiol, amido, NR⁴R⁵, C(O)NR⁴R⁵, C₁₋₆alkyl, C₁₋₆haloalkyl, C₁₋₆alkoxy and C₁₋₆alkylthio;
R⁴ and R⁵ are independently selected from H and C₁₋₃alkyl;
n is 0, 1, 2, 3 or 4;
D is a reactive group; and
X is halo.

As described above, compounds of formula (I) are able to selectively bind to hole-modified mutant Brd4 bromodomains within a tagged fusion protein. Accordingly, viewed from a further aspect, there is provided a degrader compound of formula (IA) for use in a method of studying an effect of degrading a target protein within a cell (such as the methods described above),

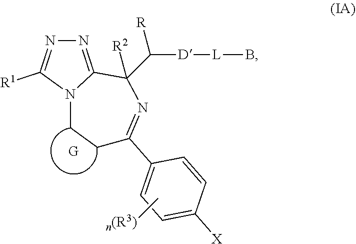

wherein G, R, R¹, R², R³, X and n are as defined above, D′ is the product of a reactive group, D (as defined above), with a pro-linker to form D′-L, L is a molecule capable of binding D′ to B and B is a molecule capable of binding to an E3 ubiquitin ligase.

Without being bound by theory, the inventors have found that molecules with analogous structures to formula (IA), but where G is a methoxy-substituted benzene ring, are unable to form a stable structure with the von Hippel-Lindau (VHL) substrate recognition subunit of E3 ligases. The inventors have found that the methoxy substituents of the benzene ring sterically clash with His110 of the VHL subunit of E3 ligases, resulting in no detectable protein degradation when a VHL binder is used. Therefore, the size of feature G is surprisingly important. When methoxy-substituted benzene is replaced with a dimethylthiophene, the resulting compounds of formula (IA) are highly effective protein degraders.

As described above, degrader compounds of formula (IA), are able to selectively bind and promote degradation of hole-modified mutant Brd4 bromodomains within a tagged fusion protein. Therefore, viewed from yet another aspect, there is provided use of the degrader compounds described above, to degrade a protein of interest, such as a protein comprising a hole-modified mutant Brd4 bromodomain tag.

### DETAILED DESCRIPTION

The present invention uses a PROTAC approach for targeted protein degradation. PROTACs comprise two active domains and a linker. One active domain is able to engage an E3 ubiquitin ligase and the other binds to a target protein, with the intention of degrading the target protein. Generally, this approach is limited to the availability of small-molecule ligands that bind to the target protein. The inventors have found that compounds of formula (I) are able to selectively bind to hole-modified mutant Brd4 bromodomains within a tagged fusion protein. Compounds of formula (I) are able to bind to any protein that may be tagged with a hole-modified mutant Brd4 bromodomain. In addition, degrader compounds of formula (IA), are able to selectively bind and promote degradation of hole-modified mutant Brd4 bromodomains within a tagged fusion protein. Therefore, degrader compounds of formula (IA) may degrade target proteins that would usually be difficult or impossible to degrade by traditional PROTAC approaches (e.g. where small-molecule ligands that bind to the target protein are not available). The degrader compounds of formula (IA), are suitable degraders to assess the functional consequences of target protein degradation in genetically engineered models.

In the discussion that follows, reference is made to a number of terms, which are to be understood to have the meanings provided below, unless a context indicates to the contrary. The nomenclature used herein for defining compounds, in particular the compounds described herein, is intended to be in accordance with the rules of the International Union of Pure and Applied Chemistry (IUPAC) for chemical compounds, specifically the “IUPAC Compendium of Chemical Terminology (Gold Book)” (see A. D. Jenkins et al., Pure & Appl. Chem., 68, 2287-2311 (1996)). For the avoidance of doubt, if an IUPAC rule is contrary to a definition provided herein, the definition herein is to prevail.

The term “comprising” or variants thereof will be understood to imply the inclusion of a stated element, integer or step, or group of elements, integers or steps, but not the exclusion of any other element, integer or step, or group of elements, integers or steps.

The term “consisting” or variants thereof will be understood to imply the inclusion of a stated element, integer or step, or group of elements, integers or steps, and the exclusion of any other element, integer or step or group of elements, integers or steps.

The term “alkyl” is well known in the art and defines univalent groups derived from alkanes by removal of a hydrogen atom from any carbon atom, wherein the term “alkane” is intended to define acyclic branched or unbranched hydrocarbons having the general formula CₙH₂ₙ₊₂, wherein n is an integer ≥1. C₁₋₄alkyl refers to any one selected from the group consisting of methyl, ethyl, n-propyl, iso-propyl, n-butyl, sec-butyl, iso-butyl and tert-butyl.

The term “haloalkyl” refers to alkyl groups in which at least one hydrogen atom has been replaced with a halo atom, such as fluoro, chloro or bromo, typically fluoro. Trifluoromethyl is an example of a haloalkyl.

The term “arene” defines monocyclic or polycyclic aromatic hydrocarbons, where “aromatic” defines a cyclically conjugated molecular entity with a stability (due to delocalisation) significantly greater than that of a hypothetical localised structure. The Hückel rule is often used in the art to assess aromatic character; monocyclic planar (or almost planar) systems of trigonally (or sometimes diagonally) hybridised atoms that contain (4n+2) π-electrons (where n is a non-negative integer) will exhibit aromatic character. The rule is generally limited to n=0 to 5.

The term “heteroarene” defines a monocyclic or polycyclic aromatic hydrocarbon comprising one or more heteroatoms.

The term “alkoxy” defines univalent groups derived from alcohols by removal of the hydrogen atom of the hydroxy group. The term “alcohols” refers to alkanes wherein one hydrogen atom has been replaced with a hydroxy group. Methoxy is an example of an C₁alkoxy group.

The term “alkylthio” defines univalent groups derived from alkylthiols by removal of the hydrogen atom of the thio group. The term “alkylthiol” refers to alkanes wherein one hydrogen atom has been replaced with a thio group. Methylthio is an example of a C₁alkylthio group.

The term “haloalkoxy” refers to alkoxy groups in which at least one hydrogen atom has been replaced with a halo atom, such as fluoro, chloro or bromo, typically fluoro. Trifluoromethoxy is an example of a C₁haloalkoxy.

The term “stereoisomer” is used herein to refer to isomers that possess identical molecular formulae and sequence of bonded atoms, but which differ in the arrangement of their atoms in space.

The term “enantiomer” defines one of a pair of molecular entities that are mirror images of each other and non-superimposable, i.e. cannot be brought into coincidence by translation and rigid rotation transformations. Enantiomers are chiral molecules, i.e. are distinguishable from their mirror image.

The term “racemic” is used herein to pertain to a racemate. A racemate defines a substantially equimolar mixture of a pair of enantiomers.

The term “diastereoisomers” (also known as diastereomers) defines stereoisomers that are not related as mirror images.

The term “solvate” is used herein to refer to a complex comprising a solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

The term “isotope” is used herein to define a variant of a particular chemical element, in which the nucleus necessarily has the same atomic number but has a different mass number owing to it possessing a different number of neutrons.

The terms “binding” or “accommodating into” when used herein in connection with interaction of a hole-modified mutant bromodomain of a fusion protein and a compound of the invention (e.g. the “bump” of compounds of the invention that is accommodated by the “hole” of hole-modified mutant Brd4 bromodomains) refer to association of the hole-modified mutant bromodomain and the compound. Association includes any attractive interaction between the hole-modified mutant bromodomain and the compound. Examples of attractive interactions include hydrogen bonding, Van der Waals forces, dipole-dipole forces, dipole-induced dipole forces, ion-dipole forces, ion-induced dipole forces and ionic bonding.

As described above, there is provided a compound of formula (I) for use in a method of studying an effect of degrading a target protein within a cell,

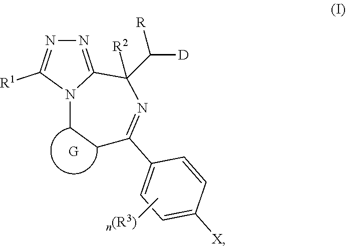

wherein
G is a 5-membered heteroarene optionally substituted with one or two substituents selected from the group consisting of methyl, halo, hydroxy, thiol, halomethyl, amino, methoxy, methylamino, dimethylamino, ethyl, haloethyl, amido, isopropyl and methylthio, or G is a 6-membered arene or heteroarene optionally substituted with methyl, halo, hydroxy and thiol;
R is C₁₋₄alkyl or C₁₋₄haloalkyl;
R¹ is any one selected from the group consisting of C₁₋₄alkyl, C₁₋₄haloalkyl, H and halo;
R² is H, C₁₋₃alkyl, C₁₋₃haloalkyl or halo;
R³ is independently selected from halo, hydroxyl, thiol, amido, NR⁴R⁵, C(O)NR⁴R⁵, C₁₋₆alkyl, C₁₋₆haloalkyl, C₁₋₆alkoxy and C₁₋₆alkylthio;
R⁴ and R⁵ are independently selected from H and C₁₋₃alkyl;
n is 0, 1, 2, 3 or 4;
D is a reactive group; and
X is halo.

As described above, and without being bound by theory, the inventors have found that molecules with analogous structures to formula (IA), but where G is a methoxy-substituted benzene ring, are unable to form a stable structure with the von Hippel-Lindau (VHL) substrate recognition subunit of E3 ligases. The inventors have found that the methoxy substituents of the benzene ring sterically clash with His110 of the VHL subunit of E3 ligases, resulting in no detectable protein degradation when a VHL binder is used. However, when methoxy-substituted benzene is replaced with a smaller group such as dimethylthiophene, the resulting compounds of formula (IA) are highly effective protein degraders. Accordingly, G of compounds (I) and (IA) is less sterically bulky than methoxybenzene.

Therefore, in some embodiments, when G is a 6-membered arene or heteroarene, it is unsubstituted. Examples of suitable 6-membered arenes and heteroarenes include benzene, pyridine, pyrimidine, pyrazine and pyridazine. In some embodiments, the 6-membered arene or heteroarene of G is benzene, pyridine, pyrimidine or pyrazine, typically benzene.

Examples of suitable 5-membered heteroarenes include thiophene, furan, pyrrole, thiazole, imidazole, pyrazole, oxazole, isothiazole and isoxazole. In some embodiments, the 5-membered heteroarene of G is any one selected from the group consisting of thiophene, furan, pyrrole and thiazole, such as thiophene.

When G is a 5-membered heteroarene, it is optionally substituted with one or two substituents selected from the group consisting of methyl, halo, hydroxy, thiol, halomethyl, amino, methoxy, methylamino, dimethylamino, ethyl, haloethyl, amido, isopropyl and methylthio. Sometimes, the 5-membered ring of G is optionally substituted with one or more substituents selected from the group consisting of methyl, halo (such as fluoro), hydroxy, thiol, halomethyl (such as trifluoromethyl) and amino. Often, the 5-membered ring of G is optionally substituted with one or more substituents selected from the group consisting of methyl, fluoro, hydroxy, thiol and trifluoromethyl. Typically, the 5-membered ring of G is optionally substituted with methyl.

In some embodiments, G is an optionally substituted 5-membered heteroarene, typically an optionally substituted thiophene. In some embodiments, G is thiophene substituted one or two times. In some embodiments, G is thiophene substituted once or twice with methyl. Typically, G is thiophene substituted twice with methyl.

As described above, X is halo, such as chloro, fluoro, bromo or iodo. In some embodiments, X is chloro.

R³ is independently selected from halo (such as fluoro), hydroxyl, thiol, amido, NR⁴R⁵, C(O)NR⁴R⁵, C₁₋₆alkyl, C₁₋₆haloalkyl (such as C₁₋₆fluoroalkyl), C₁₋₆alkoxy and C₁₋₆alkylthio, wherein R⁴ and R⁵ are independently selected from H and C₁₋₃alkyl. Often, R³ is independently selected from halo (such as fluoro), hydroxyl, thiol, amido, NH₂, N(CH₃)₂, C(O)NH₂, C(O)N(CH₃)₂, C₁₋₃alkyl, C₁₋₃haloalkyl (such as C₁₋₃fluoroalkyl), C₁₋₃alkoxy and C₁₋₃alkylthio. Often, R³ is independently selected from halo (such as fluoro), hydroxyl, thiol, amido, NH₂, N(CH₃)₂, C(O)NH₂, C(O)N(CH₃)₂, methyl, trifluoromethyl, methoxy and methylthio. Typically, R³ is independently selected from fluoro, hydroxyl, thiol, amido, NH₂, and N(CH₃)₂.

As described above, the number of R³ substituents, n, is 0, 1, 2, 3 or 4. Often, n is 0, 1 or 2, such as 0 or 1. In some embodiments, n is 0.

R¹ is any one selected from the group consisting of C₁₋₄alkyl, C₁₋₄haloalkyl (such as C₁₋₄fluoroalkyl), H and halo (such as fluoro). Often, R¹ is C₁₋₄alkyl or C₁₋₄haloalkyl (such as C₁₋₄fluoroalkyl). In some embodiments, R¹ is methyl or trifluoromethyl, typically methyl.

R² is H, C₁₋₃alkyl, C₁₋₃haloalkyl (such as C₁₋₃fluoroalkyl) or halo (such as fluoro). Often, R² is H, methyl, ethyl, trifluoromethyl or fluoro. In some embodiments, R² is H, methyl, trifluoromethyl or fluoro. Typically, R² is H.

As described above, R is C₁₋₄alkyl or C₁₋₄haloalkyl. The R group provides a “bump” that is accommodated by the “hole” of hole-modified mutant Brd4 bromodomains. Without being bound by theory, the inventors have found that compounds of formula (I) and (IA) (comprising non-hydrogen R groups) are able to bind or be accommodated into the “hole” of hole-modified mutant Brd4 bromodomains, but are unable to bind or be accommodated into the binding sites of non-modified Brd4 bromodomains. Accordingly, compounds of formula (I) and formula (IA) are able to selectively bind or selectively be accommodated into hole-modified mutant Brd4 bromodomains, whereas analogous compounds in which R is hydrogen are not selective. It is envisaged that larger R groups (such as C₅₋₈alkyl or C₅₋₈haloalkyl) could be used to bind or be accommodated into hole-modified mutant Brd4 bromodomains comprising larger holes.

Often, R is C₁₋₃alkyl or C₁₋₃fluoroalkyl. Typically, R is any one selected from the group comprising ethyl, propyl, fluoroethyl and fluoropropyl. In some embodiments, R is ethyl.

D is a reactive group. The term “reactive group” refers to any group that is capable of reacting with a second compound (typically a pro-linker compound) in order to form a bond with the second compound. Provided that D is capable of linking compound (1) with a pro-linker molecule in order to form a bond to a linker, the precise identity of D is not important and the compound of formula (I) need not be limited to specific D groups.

Often, D is any one selected from the group consisting of (CH₂)ₚC(O)OH, (CH₂)ₚC(O)Cl, (CH₂)ₚC(O)Br, (CH₂)_{q}NH₂, (CH₂)_{q}N(C₁₋₃alkyl)H, (CH₂)_{q}SH, (CH₂)_{q}OH, (CH₂)_{q}Br, (CH₂)_{q}I, (CH₂)_{q}N₃ and (CH₂)_{q}CCH; wherein p is an integer from 0 to 4 and q is an integer from 1 to 4. Often, p is 0, 1 or 2, typically 0. Often, q is 1 or 2, typically 1. In some embodiments, p is 0 and q is 1.

D is often any one selected from the group consisting of (CH₂)ₚC(O)OH, (CH₂)ₚC(O)Cl, (CH₂)ₚC(O)Br, (CH₂)_{q}NH₂, (CH₂)_{q}N(C₁₋₃alkyl)H, (CH₂)_{q}SH and (CH₂)_{q}OH. Commonly, D is any one selected from the group consisting of (CH₂)ₚC(O)OH, (CH₂)ₚC(O)Cl and (CH₂)ₚC(O)Br. In some embodiments, D is C(O)OH.

For the avoidance of doubt, groups that function in the same way as the functional groups listed above are included as equivalents of said functional groups.

For example, where D is C(O)OH, protonated variants such as C(O)*HOH (in which the carbonyl group is protonated at the oxygen atom) are included.

Where G is thiophene, it may be bound to the diazepine ring at the 2 and 3 positions or at the 3 and 4 positions. For the avoidance of doubt, the 2, 3 and 4 positions are as labelled below.

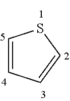

Where G is thiophene, it is typically bonded to the diazepine ring at the 2 and 3 positions, i.e. in some embodiments, the compound is of formula (II):

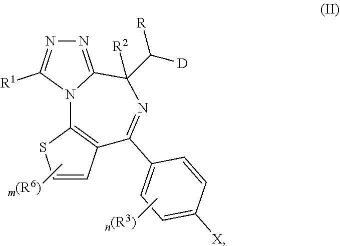

wherein R⁶ is the substituent of G (when G is a 5-membered heteroarene) and m is the number of substituents. For the avoidance of doubt, the embodiments described herein in relation to the substituents of G when G is a 5-membered heteroarene apply mutatis mutandis to R⁶ of this embodiment. For example, R⁶ may be independently selected from the group consisting of methyl, halo (such as fluoro), hydroxy, thiol, halomethyl (such as trifluoromethyl) and amino, typically methyl.

In some embodiments, R¹ is methyl, R² is hydrogen, n is 0, G is thiophene bonded to the diazepine at the 2 and 3 positions and X is chloro, i.e. the compound is of formula (III):

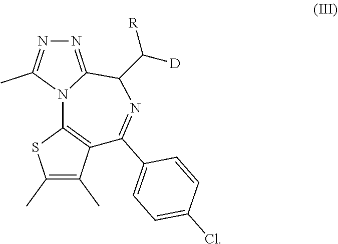

In some embodiments, R¹ is methyl, R² is hydrogen, n is 0, G is thiophene bonded to the diazepine at the 2 and 3 positions, X is chloro and D is C(O)OH, i.e. the compound is of formula (IIIa):

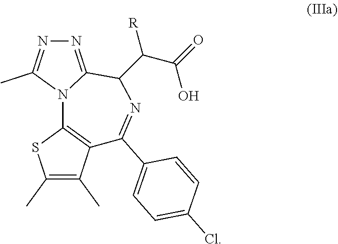

Typically, the compound is any one of formulae (IV), (IVa) and (IVb):

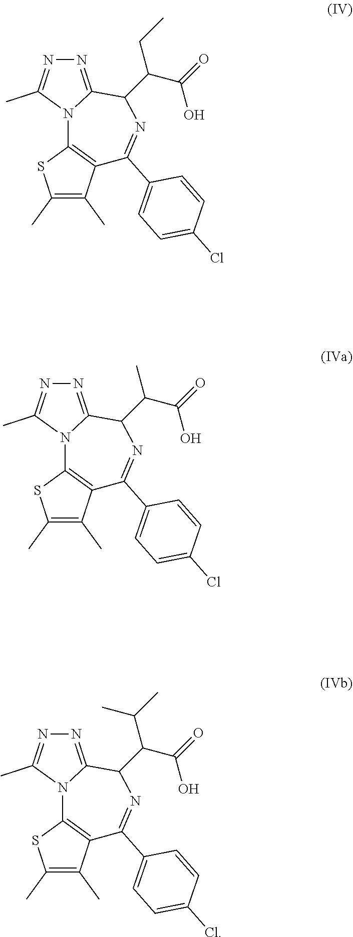 [SMILES: CCC(C(=O)O)C1N=C(c2ccc(Cl)cc2)c2c(sc(C)c2C)-n2c(C)nnc21 ; Cc1sc2c(c1C)C(c1ccc(Cl)cc1)=NC(C(C)C(=O)O)c1nnc(C)n1-2 ; Cc1sc2c(c1C)C(c1ccc(Cl)cc1)=NC(C(C(=O)O)C(C)C)c1nnc(C)n1-2]

In some embodiments, the compound is of formula (IV).

The compounds of the invention exist in different diastereoisomeric forms owing to chirality at the carbon atoms identified with asterisks below.

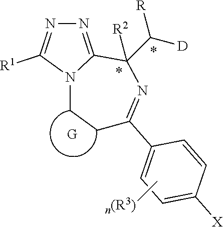

All stereoisomeric forms and mixtures thereof, including enantiomers and racemic mixtures, are included within the scope of the invention. Individual stereoisomers of compounds of formula I, i.e. compounds comprising less than 5%, 2% or 1% (e.g. less than 1%) of the other stereoisomer, are also included. Mixtures of stereoisomers in any proportion, for example a racemic mixture comprising substantially equal amounts of two enantiomers, are also included within the invention.

Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which do not cause racemisation or epimerisation.

Often, the compounds of the invention are enantiomerically pure. Often, the compound is of formula (Ia):

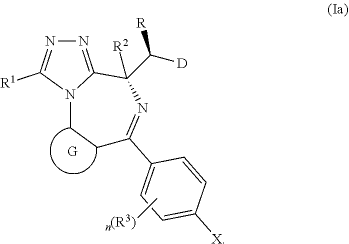

Typically, the compound is any one of formulae (V), (Va) and (Vb):

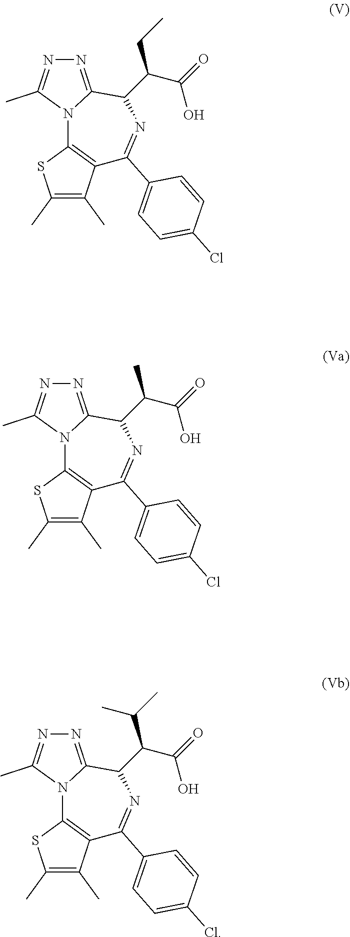

In some embodiments, the compound is of formula (V).

As described above, compounds of formula (I) are able to selectively bind or be accommodated into hole-modified mutant Brd4 bromodomains within a tagged protein.

Accordingly, viewed from another aspect, there is provided a degrader compound of formula (IA) for use in use in a method of studying an effect of degrading a target protein within a cell,

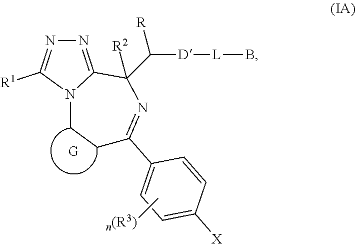

wherein G, R, R¹, R², R³, X and n are as defined above, D′ is the product of a reactive group, D (as defined above), with a pro-linker to form D′-L, L is a molecule capable of binding D′ to B and B is a molecule capable of binding to an E3 ubiquitin ligase.

For the avoidance of doubt, the embodiments described herein in relation to formula (I) apply mutatis mutandis to formula (IA). For example, the degrader compound may be any one of formulae (II), (III), (IIIa), (IV), (IVa), (IVb), (IVc), (Ia), (V), (Va), (Vb) or (Vc), provided that D or C(O)OH is replaced with D′-L-B or C(O)-L-B, respectively.

The inventors have found that molecules with analogous structures to formula (IA), but where G is a methoxy-substituted benzene ring, are unable to form a stable structure with the von Hippel-Lindau (VHL) substrate recognition subunit of E3 ligases.

Without being bound by theory, the methoxy substituents of the benzene ring sterically clash with His110 of the VHL subunit of E3 ligases, resulting in no detectable protein degradation when a VHL binder is used. Therefore, the size of feature G is surprisingly important. When methoxy-substituted benzene is replaced with a dimethylthiophene, the resulting compounds of formula (IA) are highly effective protein degraders.

As described above, D′ is the product of a reactive group, D (as defined above), with a pro-linker to form D′-L. A pro-linker is defined herein as a molecule that is capable of reacting with D′ to form D′-L, where L is a molecule capable of binding D′ to B. Provided that D′ is capable of binding to L, the precise identity of D′ is not important and the compound of formula (IA) need not be limited to specific D′ groups.

In some embodiments, however, D′ is any one selected from the group consisting of (CH₂)ₚC(O), (CH₂)_{q}NH, (CH₂)_{q}S, (CH₂)_{q}O, (CH₂)_{q} and 1,2,3-triazolylene, wherein p is an integer from 0 to 4 and q is an integer from 1 to 4. Often, D′ is any one selected from the group consisting of (CH₂)ₚC(O), (CH₂)_{q}NH, (CH₂)_{q}S, and (CH₂)_{q}O, typically (CH₂)ₚC(O).

Often, p is an integer from 0 to 2 and q is 1 or 2. In some embodiments, p is 0 and q is 1. Accordingly, in some embodiments, D′ is C(O).

As described above, L is a molecule capable of binding D′ to B and may be any one of the linkers described in R. I. Troup, C. Fallan and M. G. J. Baud, Explo. Target Anitiumor Ther. 2020, 1, 273-312. In some embodiments, L is of formula (VIA):

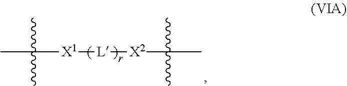

wherein
the wavy lines indicate the positions of attachment to D′ and B;
X¹ is optionally present and is any one selected from the group consisting of O(CH₂)ₛ, NH(CH₂)ₛ and C(O)(CH₂)ₛ;
X² is optionally present and is selected from the group consisting of O(CH₂)ᵤC(O), (CH₂)ᵤNH, (CH₂)ᵤO and (CH₂)ᵤC(O);
L′ is selected from the group consisting of O(CH₂)ₜ, CH₂, alkynylene, triazolylene, piperazinylene and piperidinylene;
s is an integer from 0 to 4, u is an integer from 1 to 4 and t is an integer from 1 to 4.

For the avoidance of doubt, X¹ of formula (VIA) binds to D′ and X² binds to B. Where X¹ is absent, D′ binds directly to (L′)ᵣ, and where X² is absent, (L′)ᵣ binds directly to B. In some embodiments, X¹ and X² are present.

In some embodiments, X¹ is any one selected from the group consisting of O(CH₂)ₛ and HN(CH₂)ₛ. Often, s is 0 to 2, typically 2. In some embodiments, X¹ is O(CH₂)₂.

L′ is often selected from the group consisting of O(CH₂)ₜ, CH₂ and alkynylene, where t is an integer form 1 to 4. Typically, L′ is O(CH₂)ₜ. t is often 2 or 3, thus in some embodiments, L′ is O(CH₂)₂ or O(CH₂)₃.

In some embodiments, X² is O(CH₂)ᵤC(O) or (CH₂)ᵤNH. Often, u is 1 or 2, typically 1. Therefore, in some embodiments, X² is OCH₂C(O) or CH₂NH.

In some embodiments, the degrader compound is in accordance with any of the teaching above, wherein B is selected from the following structures represented by any one of formulae (VIIA) to (XVA):

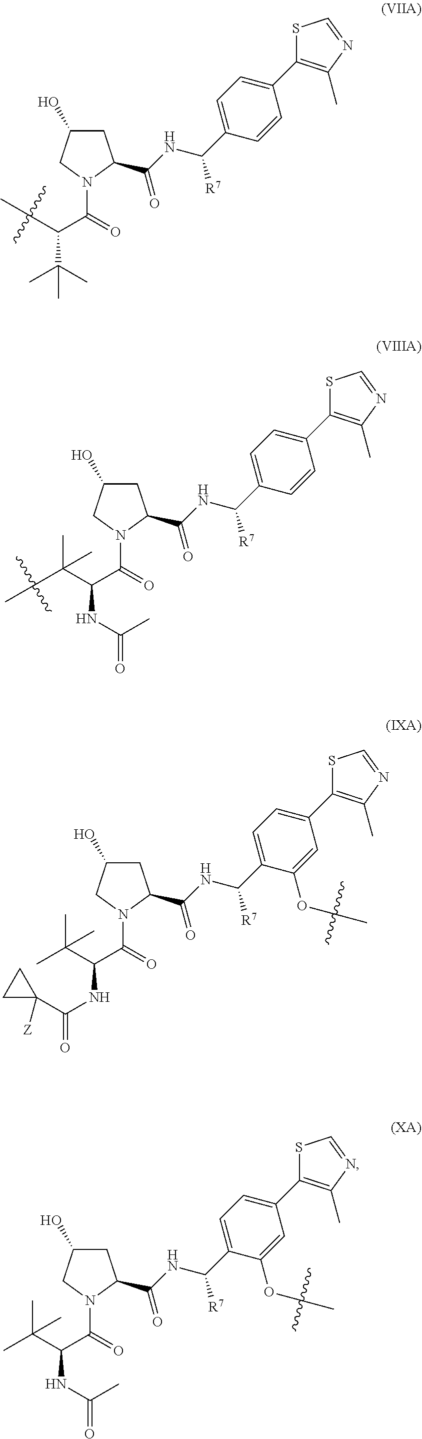

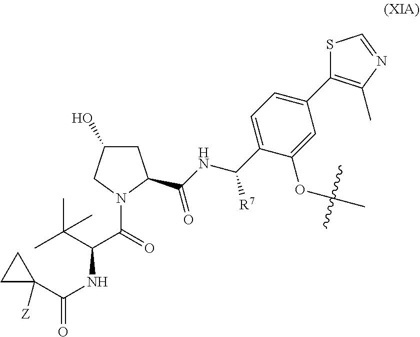

wherein R⁷ is H or methyl and Z is F or CN, such as F.

In some embodiments, B is the structure represented by any one of formulae (VIIA) to (IXA). In some embodiments B is the structure represented by formula (VIIA).

In some embodiments of structures (VIIA), (VIIIA), (IXA), (XA) and (XIA), R⁷ is H.

In some embodiments, the degrader compound is any one of formulae (XIIA) to (XIVA):

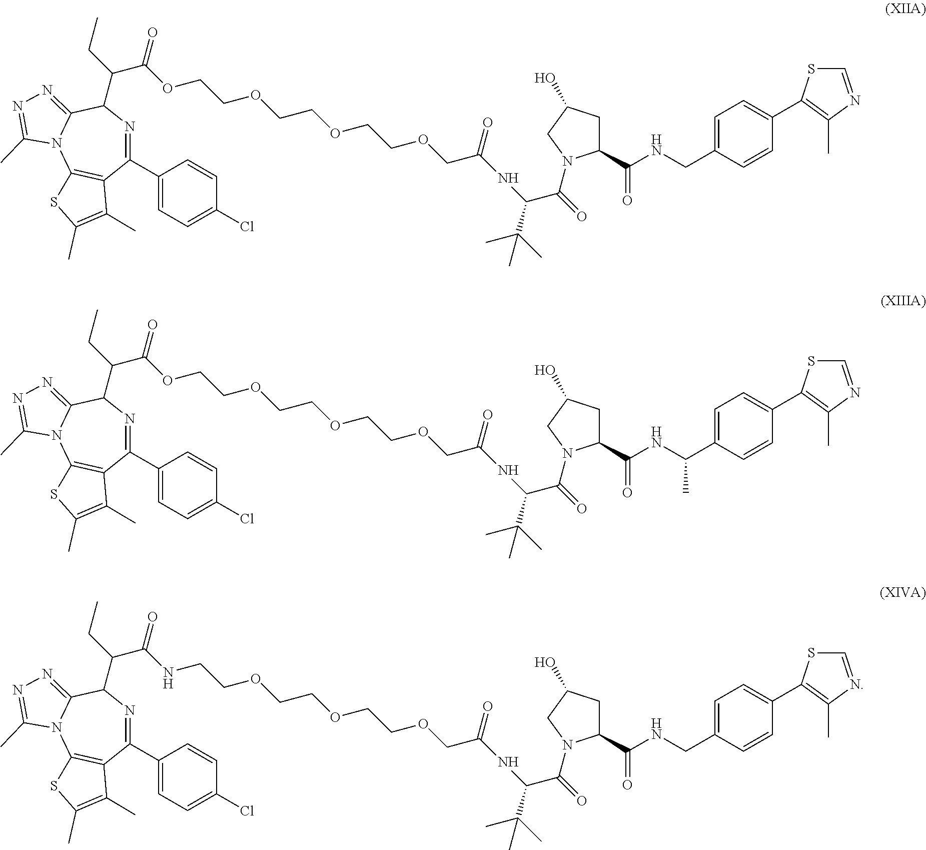 [SMILES: CCC(C(=O)OCCOCCOCCOCC(=O)N[C@H](C(=O)N1C[C@H](O)C[C@H]1C(=O)NCc1ccc(-c2scnc2C)cc1)C(C)(C)C)C1N=C(c2ccc(Cl)cc2)c2c(sc(C)c2C)-n2c(C)nnc21 ; CCC(C(=O)OCCOCCOCCOCC(=O)N[C@H](C(=O)N1C[C@H](O)C[C@H]1C(=O)N[C@@H](C)c1ccc(-c2scnc2C)cc1)C(C)(C)C)C1N=C(c2ccc(Cl)cc2)c2c(sc(C)c2C)-n2c(C)nnc21 ; CCC(C(=O)NCCOCCOCCOCC(=O)N[C@H](C(=O)N1C[C@H](O)C[C@H]1C(=O)NCc1ccc(-c2scnc2C)cc1)C(C)(C)C)C1N=C(c2ccc(Cl)cc2)c2c(sc(C)c2C)-n2c(C)nnc21]

In some embodiments, the degrader compound is of formula (XIIA).

In more specific embodiments, the degrader compound is any one of formulae (XVA) to (XVIIA):

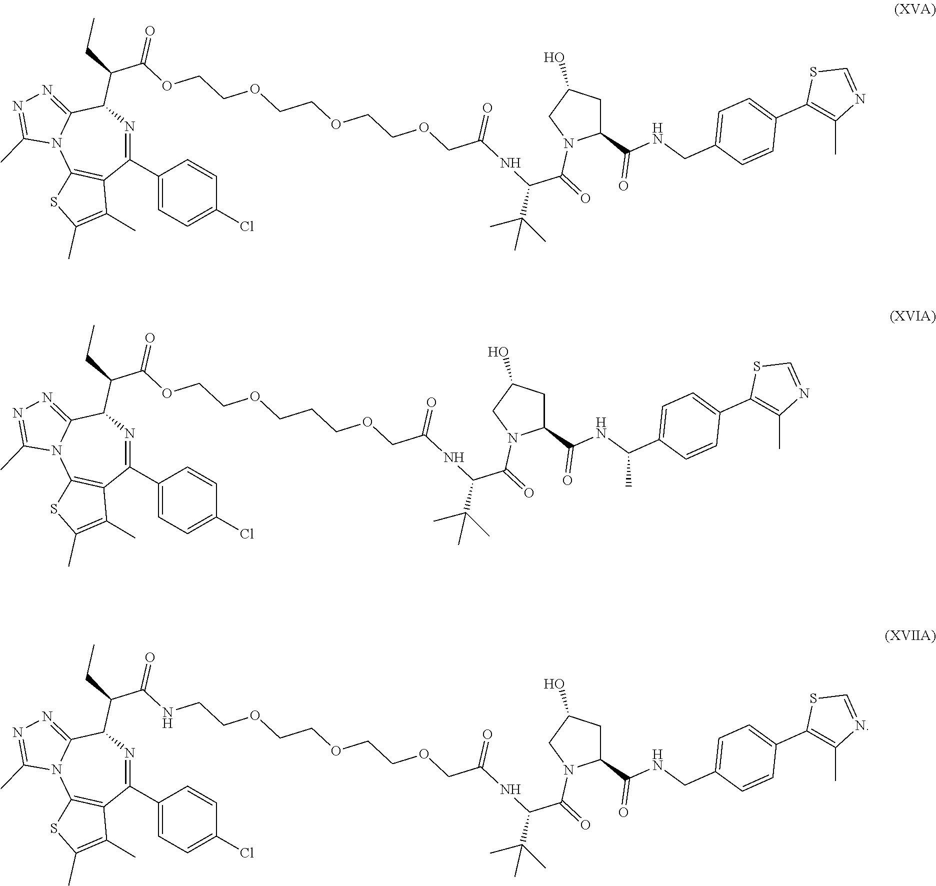 [SMILES: CC[C@@H](C(=O)OCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)OCCOCCCOCC(=O)N[C@H](C(=O)N1C[C@H](O)C[C@H]1C(=O)N[C@@H](C)c1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)NCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21]

In one embodiment, the degrader compound is of formula (XVA).

The degrader compounds of the present disclosure may find particular use in degrading proteins, in vitro or in vivo. Thus, the degrader compounds may be used in a method of degrading target proteins, the method comprising contacting the degrader compound with a suitable hole-modified bromodomain tagged fusion protein, for a period of time, in order to degrade the target protein which is part of the fusion protein.

As described above, the compounds of the present disclosure (comprising non-hydrogen R groups) are able to selectively bind or accommodate into hole-modified mutant bromodomains (such as mutant Brd4 bromodomains), whereas analogous compounds in which R is hydrogen are not selective. By “selectively bind” is meant that the compounds bind or are accommodated into hole-modified mutant bromodomains (such as mutant Brd4 bromodomains) more strongly (e.g. with a greater association constant and a smaller dissociation constant) than to bromodomains that are not hole-modified mutants, such as those of BET proteins, e.g. the endogenous proteins Brd2, Brd3, and Brd4. Advantageously, the compounds of the invention are far more selective for hole-modified mutant bromodomains (such as mutant Brd4 bromodomains) than wild-type bromodomains (such as BET bromodomains) that are not hole-modified mutants. For example, degradation of proteins comprising bromodomains, such as proteins Brd2, Brd3 and Brd4 comprising BET bromodomains, that are not hole-modified mutants, by the degrader compounds of the invention may not be detected (e.g. there may not be a reduction in the signal corresponding to proteins comprising bromodomains that are not hole-modified mutants, when analysed by Western blot methods).

Advantageously, the degrader compounds of the invention are potent degraders of target proteins (when the target proteins are part of hole-modified bromodomain tagged fusion proteins). For example, the degrader compounds of the invention may exhibit on-target degradation potency (half-maximal degradation concentration (DC₅₀)) values of 0.001 to 500 nM, such as 0.01 to 400 nM or 0.1 to 300 nM.

Advantageously, the degrader compounds of the invention are highly effective degraders of target proteins (when the target proteins are part of hole-modified bromodomain tagged fusion proteins). For example, the degrader compounds of the invention may exhibit efficacies (maximum degradation (Dₘₐₓ)) values of 50 to 100%, such as 65 to 100% or 70 to 100%.

Furthermore, the degrader compounds of the invention may act quickly to degrade target proteins (when the target proteins are part of hole-modified bromodomain tagged fusion proteins). For example, when in the presence of the degrader compound, the target protein half-life (t_{1/2}) may be less than 3 hours, such as 0.001 to 150 minutes or 0.01 to 140 minutes.

Methods suitable for the analysis of the degradation of proteins, such as Western blot methods and methods to calculate DC₅₀, Dₘₐₓ, and t_{1/2} are described in the experimental section below.

Any discussion herein of documents, acts, materials, devices, articles or the like is not to be taken as an admission that any or all of these matters form part of the prior art base or were common general knowledge in the field relevant to the present disclosure as it existed before the priority date of each claim of this application.

It will be appreciated by those skilled in the art that numerous variations and/or modifications may be made to the invention as described herein without departing from the scope of the invention as described. The present embodiments are therefore to be considered for descriptive purposes and are not restrictive, and are not limited to the extent of that described in the embodiment. The person skilled in the art is to understand that the present embodiments may be read alone, or in combination, and may be combined with any one or a combination of the features described herein.

The subject-matter of each patent and non-patent literature reference cited herein is hereby incorporated by reference in its entirety.

The invention may be further understood with reference to the following non-limiting clauses:
1. A method of studying an effect of degrading a target protein within a cell, the method comprising:
   endogenously expressing a fusion protein comprising the target protein, fused to a polypeptide comprising a hole-modified mutant bromodomain;
   contacting the fusion protein with a degrader compound comprising a segment capable of binding specifically to the hole-modified mutant bromodomain of the fusion protein, a linker, and a von Hippel-Lindau (VHL) ligand capable of binding to a protein complex comprising elongin B, elongin C and cullin-2, which possesses E3 ubiquitin ligase activity; and
   observing any effect upon the cell of degrading the target protein.
2. The method according to clause 1, wherein the target protein is endogenously tagged with one or more hole-modified mutant bromodomain(s) from the Bromo and Extraterminal domain (BET) proteins, Brd2, Brd3, Brd4 and BrdT, or a bromodomain containing fragment thereof.
3. The method according to clause 2, wherein the mutation occurs in one of more bromodomains which are present in the protein, or a bromodomain containing fragment thereof.
4. The method according to clause 2 or clause 3, wherein the hole-modified mutant bromodomain is a Brd4_{BD2}L387A, Brd4_{BD2}L387V, Brd4_{BD1}L94A, Brd4_{BD1}L94V, Brd2_{BD2}L383A, Brd2_{BD2}L383V, Brd2_{BD1}L110A, Brd2_{BD1}L110V, Brd3_{BD2}L344A, Brd3_{BD2}L344V, Brd3_{BD1}L70A, Brd3_{BD1}L70V, Brd4_{BD2}L306A, Brd4_{BD2}L306V, Brd4_{BD1}L63A, or Brd4_{BD1}L63V bromodomain.
5. The method according to clause 2 or clause 3, wherein the hole-modified mutant Brd4 bromodomain comprises a Brd4_{BD2}L387A or Brd4_{BD2}L387V bromodomain.
6. A nucleic acid sequence which encodes a fusion protein, for use in a method according to any one preceding clause, the fusion protein comprising a target protein, fused to a polypeptide comprising a hole-modified mutant bromodomain as defined herein, the nucleic acid sequence comprising a nucleic acid sequence encoding the target protein and having a 5′- or 3′-in-frame insertion of a nucleic acid encoding the polypeptide comprising a hole-modified mutant bromodomain which, when expressed, results in a fusion protein which is capable of being bound by a degrader compound as described herein.
7. A vector, such as a plasmid or virus vector, comprising the nucleic acid sequence according to clause 6.
8. A cell (including a somatic, embryonic stem, or induced pluripotent cell), the genome of which has been modified to express the nucleic acid sequence of clause 6.
9. A non-human animal comprising, or derived from a cell according to clause 8.
10. A compound of formula (I), for use in a method of studying an effect of degrading a target protein within a cell,

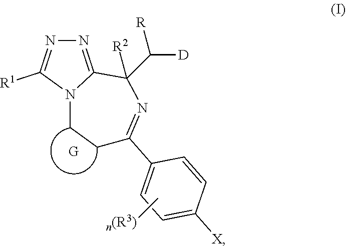

wherein
G is a 5-membered heteroarene optionally substituted with one or two substituents selected from the group consisting of methyl, halo, hydroxy, thiol, halomethyl, amino, methoxy, methylamino, dimethylamino, ethyl, haloethyl, amido, isopropyl, and methylthio, or G is a 6-membered arene or heteroarene optionally substituted with methyl, halo, hydroxy and thiol;
R is C₁₋₄alkyl or C₁₋₄haloalkyl;
R¹ is any one selected from the group consisting of C₁₋₄alkyl, C₁₋₄haloalkyl, H and halo;
R² is H, C₁₋₃alkyl, C₁₋₃haloalkyl or halo;
R³ is independently selected from halo, hydroxyl, thiol, amido, NR⁴R⁵, C(O)NR⁴R⁵, C₁₋₆alkyl, C₁₋₆haloalkyl, C₁₋₆alkoxy and C₁₋₆alkylthio;
R⁴ and R⁵ are independently selected from H and C₁₋₃alkyl;
n is 0, 1, 2, 3 or 4;
D is a reactive group; and
X is halo.
11. The compound for the use of clause 10, wherein the method is according to any one of clauses 1 to 5.
12. The compound for the use of clause 10 or clause 11, wherein the 6-membered arene or heteroarene of G is unsubstituted.
13. The compound for the use of any one of clauses 10 to 12, wherein the 6-membered arene or heteroarene of G is benzene, pyridine, pyrimidine or pyrazine.
14. The compound for the use of any one of clauses 10 to 12, wherein the 6-membered arene of G is benzene.
15. The compound for the use of any one of clauses 10 to 14, wherein the 5-membered heteroarene of G is any one selected from the group consisting of thiophene, furan, pyrrole and thiazole.
16. The compound for the use of any one of clauses 10 to 15, wherein G is a 5-membered heteroarene.
17. The compound for the use of clause 16, wherein G is thiophene.
18. The compound for the use of clause 16 or clause 17, wherein G is substituted with one or more substituents selected from the group consisting of methyl, halo, hydroxy, thiol, halomethyl, amino, methoxy, methylamino, dimethylamino, ethyl, haloethyl, amido, isopropyl, tert-butyl and methylthio.
19. The compound for the use of clause 16 or clause 17, wherein G is substituted with one or more substituents selected from the group consisting of methyl, halo, hydroxy, thiol, halomethyl and amino.
20. The compound for the use of clause 16 or clause 17, wherein G is substituted with one or more substituents selected from the group consisting of methyl, fluoro, hydroxy, thiol and fluoromethyl.
21. The compound for the use of clause 16 or clause 17, wherein G is substituted with methyl.
22. The compound for the use of any one of clauses 10 to 21, wherein G is substituted one or two times.
23. The compound for the use of any one of clauses 10 to 21, wherein G is substituted two times.
24. The compound for the use of any one of clauses 10 to 23, wherein X is chloro.
25. The compound for the use of any one of clauses 10 to 24, wherein n is 0.
26. The compound for the use of any one of clauses 10 to 25, wherein R¹ is any one selected from the group consisting of C₁₋₄alkyl and C₁₋₄fluroralkyl.
27. The compound for the use of any one of clauses 10 to 26, wherein R¹ is methyl or trifluoromethyl.
28. The compound for the use of any one of clauses 10 to 27, wherein R¹ is methyl.
29. The compound for the use of any one of clauses 10 to 28, wherein R² is H, methyl, trifluoromethyl or fluoro.
30. The compound for the use of any one of clauses 10 to 29, wherein R² is H.
31. The compound for the use of any one of clauses 10 to 30, wherein R³ is independently selected from fluoro, hydroxyl, thiol, amido, NR⁴R⁵, C(O)NR⁴R⁵, C₁₋₄alkyl, C₁₋₄fluoroalkyl, C₁₋₄alkoxy and C₁₋₄alkylthio.
32. The compound for the use of any one of clauses 10 to 31, wherein R is C₁₋₃alkyl or C₁₋₃fluoroalkyl.
33. The compound for the use of any one of clauses 10 to 31, wherein R is C₂₋₃alkyl or C₂₋₃fluoroalkyl.
34. The compound for the use of any one of clauses 10 to 31, wherein R is ethyl.
35. The compound for the use of any one of clauses 10 to 34, wherein D is any one selected from the group consisting of (CH₂)ₚC(O)OH, (CH₂)^{p}C(O)Cl, (CH₂)ₚC(O)Br, (CH₂)_{q}NH₂, (CH₂)_{q}N(C₁₋₃alkyl)H, (CH₂)_{q}SH, (CH₂)_{q}OH, (CH₂)_{q}Br, (CH₂)_{q}I, (CH₂)_{q}N₃ and (CH₂)_{q}CCH; wherein p is an integer from 0 to 4 and q is an integer from 1 to 4.
36. The compound for the use of clause 35, wherein p is 0 and q is 1.
37. The compound for the use of any one of clauses 10 to 34, wherein D is C(O)OH.
38. The compound for the use of any one of clauses 17 to 23, wherein the compound is of formula (II)

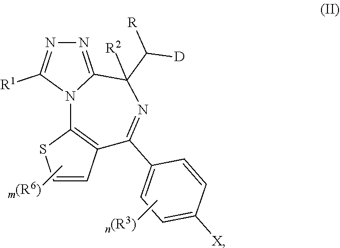

wherein R⁶ is the substituent of G, m is the number of substituents, and R, R¹, R², R³, D, X and n are as defined in any one of clauses 10 and 24 to 37.
39. The compound for the use of clause 10, wherein the compound is of formula (III)

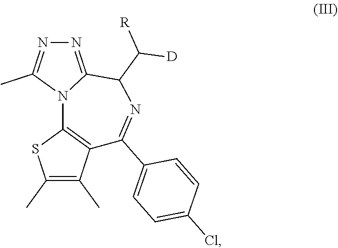

wherein R and D are as defined in any one of clauses 10 and 32 to 37.
40. The compound for the use of clause 10, wherein the compound is of formula (IV)

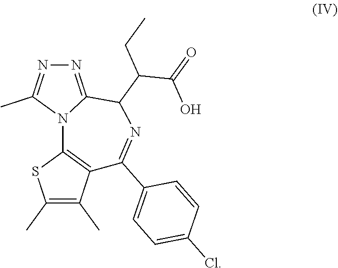 [SMILES: CCC(C(=O)O)C1N=C(c2ccc(Cl)cc2)c2c(sc(C)c2C)-n2c(C)nnc21]

41. The compound for the use of clause 10, wherein the compound is of formula (V)

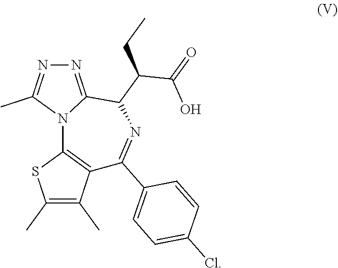

42. A degrader compound of formula (IA) for use in a method of studying an effect of degrading a target protein within a cell

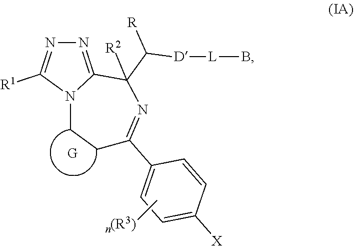

wherein G, R, R¹, R², R³, X and n are as defined in any one of clauses 10 and 12 to 37, D′ is the product of a reactive group, D, with a pro-linker to form D′-L, L is a molecule capable of binding D′ to B and B is a molecule capable of binding to an E3 ubiquitin ligase.
43. The degrader compound for the use of clause 42, wherein the method is according to any one of clauses 1 to 5.
44. The degrader compound for the use of clause 42 or clause 43, wherein D′ is any one selected from the group consisting of (CH₂)ₚC(O), (CH₂)_{q}NH, (CH₂)_{q}S, (CH₂)_{q}O, (CH₂)q and 1,2,3-triazolylene, wherein p is an integer from 0 to 4 and q is an integer from 1 to 4.
45. The degrader compound for the use of clause 44, wherein p is 0 and q is 1.
46. The degrader compound for the use of any one of clauses 42 to 45, wherein D′ is C(O).
47. The degrader compound for the use of any one of clauses 42 to 46, wherein the degrader compound is of formula (IIA)

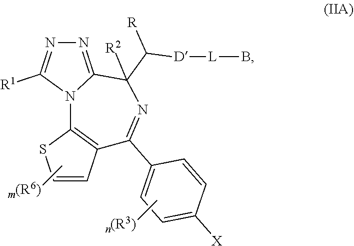

wherein R⁶ is the substituent of G, m is the number of substituents, and R, R¹, R², R³, X and n are as defined in any one of clauses 10 and 18 to 37.
48. The degrader compound for the use of any one of clauses 42 to 46, wherein the degrader compound is of formula (IIIA)

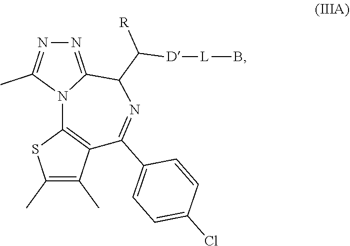

wherein R is as defined in any one of clauses 10 and 32 to 34.
49. The degrader compound for the use of clause 42 or clause 43, wherein the degrader compound is of formula (IVA)

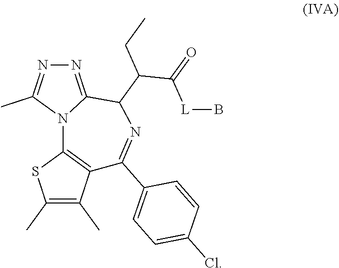

50. The degrader compound for the use of clause 42 or clause 43, wherein the degrader compound is of formula (VA)

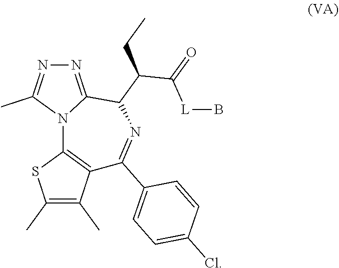

51. The degrader compound for the use of any one of clauses 42 to 50, wherein L is of formula (VIA)

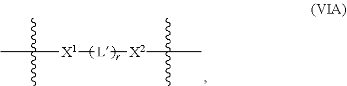

wherein
the wavy lines indicate the positions of attachment;
X¹ is optionally present and is any one selected from the group consisting of O(CH₂)ₛ, NH(CH₂)ₛ and C(O)(CH₂)ₛ;
X² is optionally present and is selected from the group consisting of O(CH₂)ᵤC(O), (CH₂)ᵤNH, (CH₂)ᵤO and (CH₂)ᵤC(O);
L′ is selected from the group consisting of O(CH₂)ₜ, CH₂, alkynylene, triazolylene, piperazinylene and piperidinylene;
s is an integer from 0 to 4, u is an integer from 1 to 4 and t is an integer from 1 to 4.
52. The degrader compound for the use of clause 51, wherein X¹ and X² are present.
53. The degrader compound for the use of clause 51 or clause 52, wherein X¹ is any one selected from the group consisting of O(CH₂)ₛ and HN(CH₂)ₛ.
54. The degrader compound for the use of any one of clauses 51 to 53, wherein s is 2.
55. The degrader compound for the use of any one of clauses 51 to 53, wherein X¹ is O(CH₂)₂.
56. The degrader compound for the use of any one of clauses 51 to 55, wherein L′ is O(CH₂)t.
57. The degrader compound for the use of any one of clauses 51 to 56, wherein t is 2 or 3.
58. The degrader compound for the use of any one of clauses 51 to 57, wherein X² is O(CH₂)ᵤC(O) or (CH₂)ᵤNH.
59. The degrader compound for the use of any one of clauses 51 to 58, wherein u is 1.
60. The degrader compound for the use of any one of clauses 42 to 59, wherein B is any one of the structures represented by any one of formulae (VIIA) to (XIIIA):

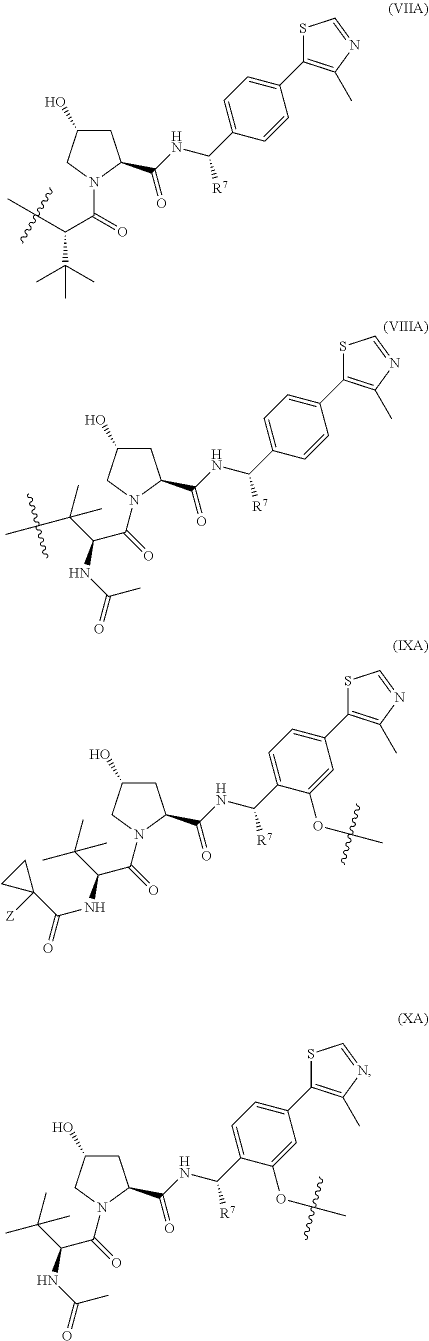

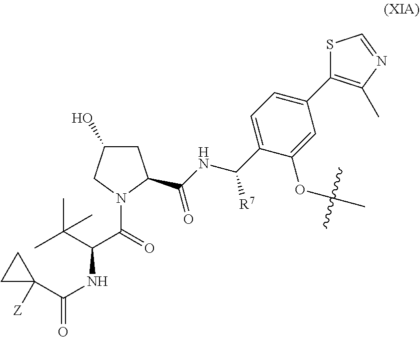

wherein R⁷ is H or methyl and Z is F or CN.
61. The degrader compound for the use of clause 60, wherein B is the structure represented by any one of formulae (VIIA) to (IXA).
62. The degrader compound for the use of clause 60, wherein B is the structure represented by formula (VIIA).
63. The degrader compound for the use of any one of clauses 60 to 62, wherein R⁷ is H.
64. The degrader compound for the use of any one of clauses 42 to 63, wherein the degrader compound is any one of formulae (XIXA) to (XXIA):

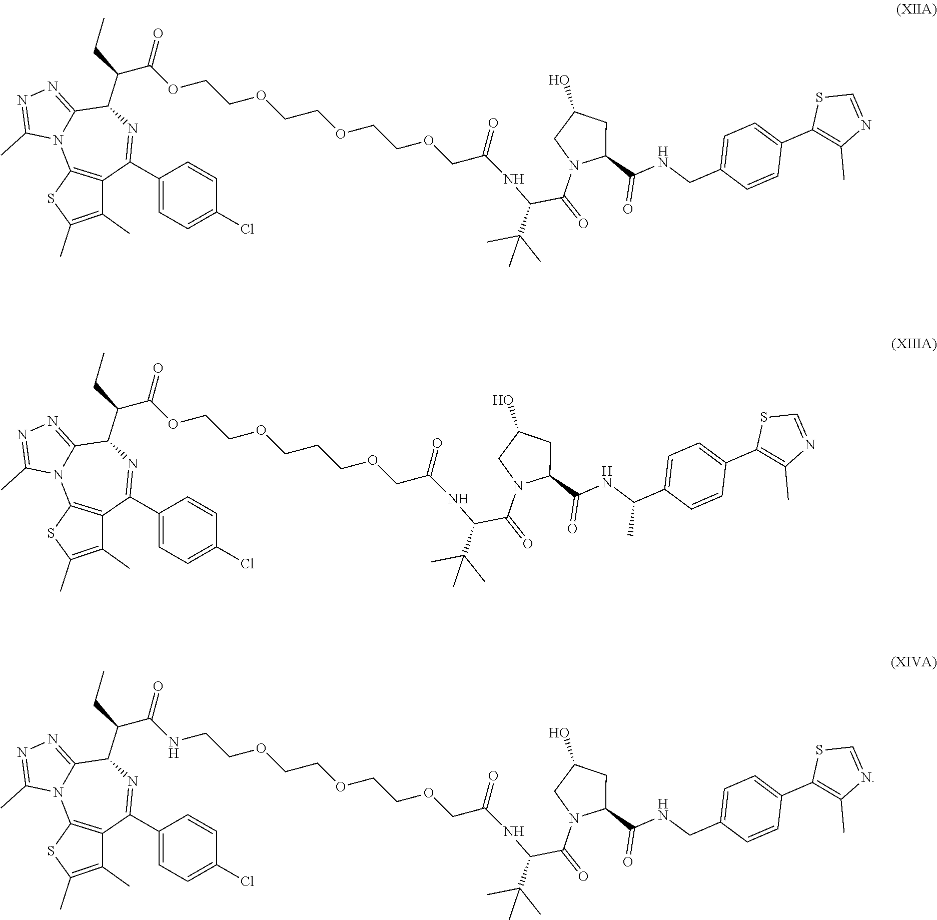 [SMILES: CC[C@@H](C(=O)OCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)OCCOCCCOCC(=O)N[C@H](C(=O)N1C[C@H](O)C[C@H]1C(=O)N[C@@H](C)c1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)NCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21]

65. The degrader compound for the use of clause 64, wherein the degrader compound is of formula (XIIA).
66. Use of a degrader compound, as defined in any one of clauses 42 to 65, to degrade a target protein.
67. The use of clause 66, wherein the target protein is endogenously tagged with a polypeptide comprising one or more hole-modified mutant Brd4 bromodomain(s).
68. The use of clause 67, wherein the hole-modified mutant Brd4 bromodomain is a Brd4_{BD2}L387A, Brd4_{BD2}L387V, Brd4_{BD1} L94A, Brd4_{BD1}L94V, Brd2_{BD2}L383A, Brd2_{BD2}L383V, Brd2_{BD1}L110A, Brd2_{BD1}L110V, Brd3_{BD2}L344A, Brd3_{BD2}L344V, Brd3_{BD1}L70A, Brd3_{BD1}L70V, Brd4_{BD2}L306A, Brd4_{BD2}L306V, Brd4_{BD1}L63A, or Brd4_{BD1}L63V bromodomain.
69. The use of clause 67, wherein the hole-modified mutant Brd4 bromodomain is a Brd4_{BD2}L387A or Brd4_{BD2}L387V bromodomain.
70. The use of clause 67, wherein the hole-modified mutant Brd4 bromodomain is a Brd4_{BD2}L387A bromodomain.
71. The use of any one of clauses 66 to 70, wherein the protein is a BET protein.
72. The use of clause 71, wherein the BET protein is a Brd2 protein.
73. A protein comprising one or more Brd4 bromodomain(s), each comprising a L387A mutation.
74. The protein of clause 73, wherein the protein is as defined in clause 71 or clause 72.

The present disclosure will now be further defined by way of example and with reference to the following figures which show:

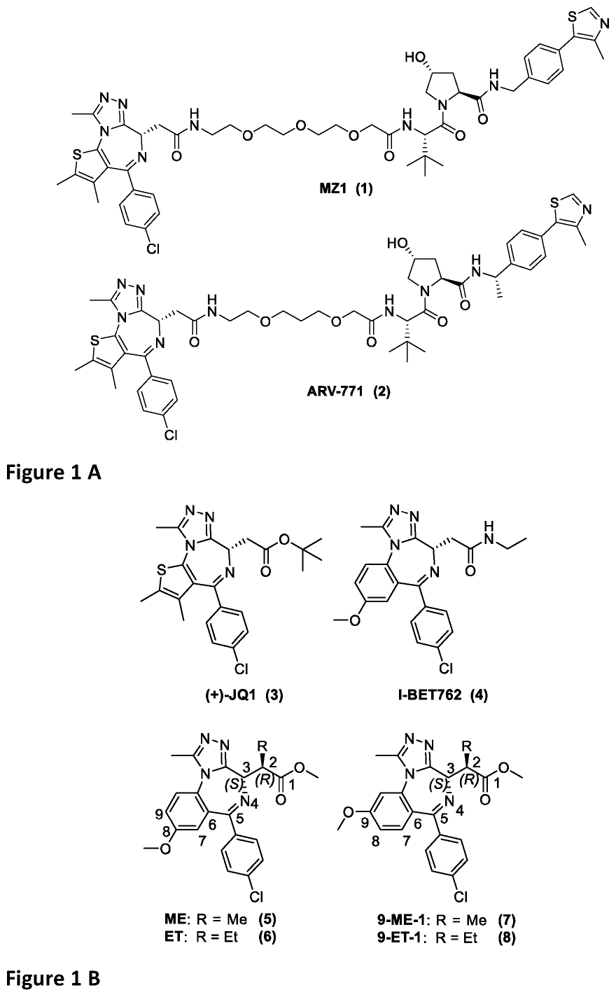

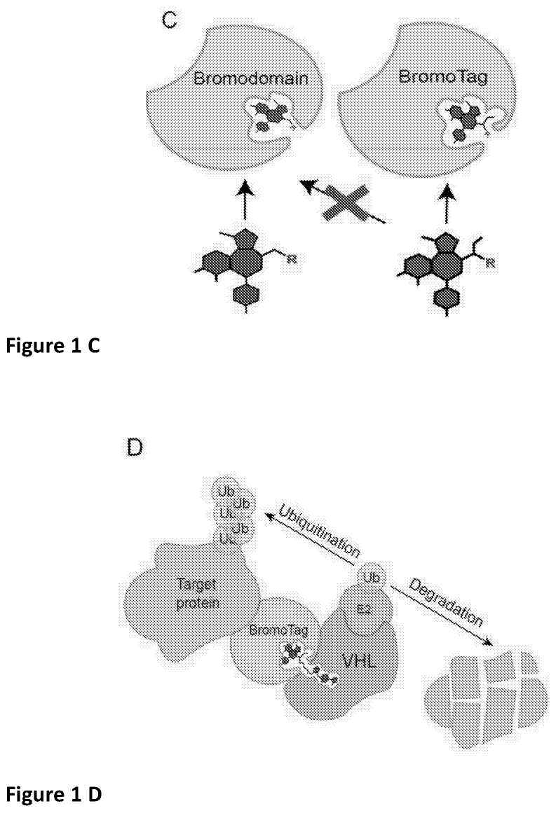

FIG. 1. (A) Pan-selective BET degraders, MZ1 and ARV-771. (B) Pan-selective BET inhibitors, (+)-JQ1 and I-BET762 (top). Allele specific bumped-BET inhibitors, ME, ET, 9-ME-1 and 9-ET-1 (bottom). (C) Tailoring the ‘Bump-and-hole’ approach to BET bromodomains to produce a high affinity selective paring of that can be utilised as a degron system. (D) Conceptualisation of the BromoTag degron approach.

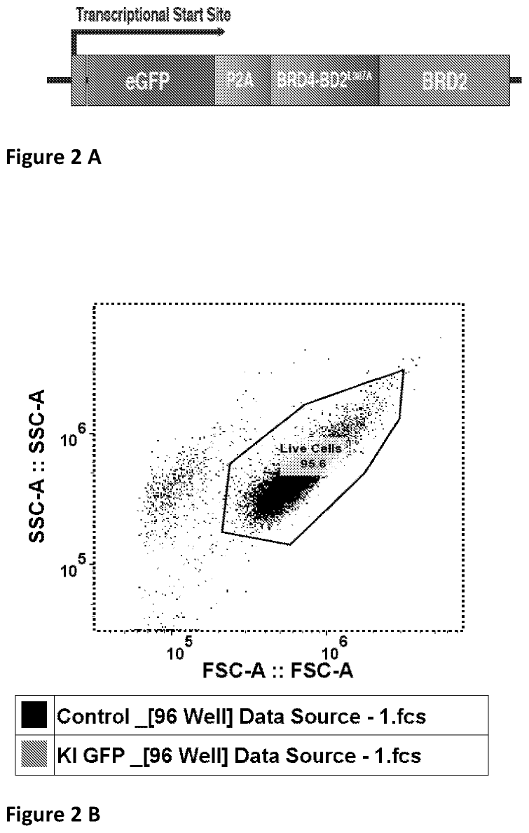

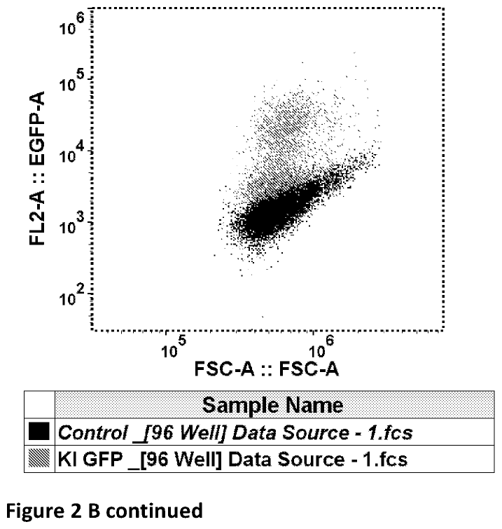

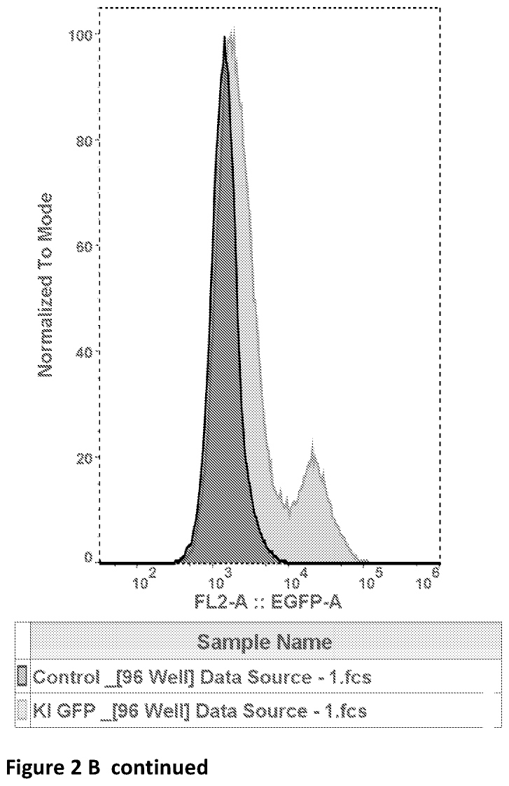

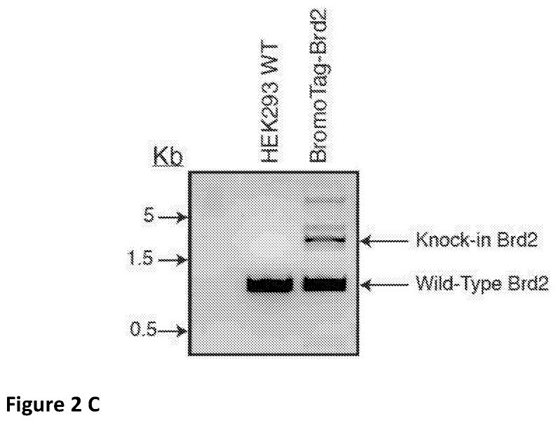

FIG. 2. Design and development of a heterozygous knock-in BromoTag-Brd2 HEK293 cell line. (A) Design of the knock-in construct used in the development of the CRISPR construct. (B) FACS single cell sort of HEK293 cells based on GFP expression. Successive single cells were sorted into individual wells of a 96-well plate. (C) Junction PCR using genomic DNA of an expanded GFP expressing clone paired against parental HEK293. (D) Western blot demonstrating selectivity of the polyclonal Brd4BD2^{L387A}. antibody.

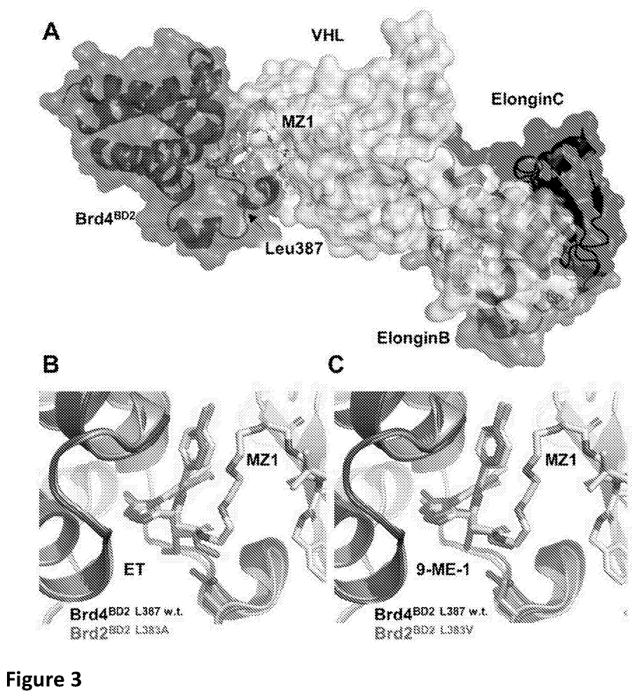

FIG. 3. (A) Ternary complex between Brd4^{BD2} (green, cartoon/surface representation), MZ1 (7, stick, grey carbons) and VCB (VHL: blue; ElonginC: pink; ElonginB—pale orange; cartoon/surface representations). Leu387 (stick, green) highlighted with arrow (PDB code: 5T35). Alignment of Brd4^{BD2} (pale green, cartoon representation, 5T35) with (B) Brd2^{BD2 L383A} (orange, cartoon representation, 4QEW) and (C) Brd2^{BD2 L383}V (yellow, cartoon representation, 503C), co crystalised with MZ1 (7, stick, grey carbons), ET (4, stick, pink carbons) and 9-ME-1 (5, stick, blue carbons) respectively. Brd4^{BD2 W.T}. Leu387 (stick, pale green carbons) and mutants Brd2^{BD2 L383A}Ala383 (stick, orange carbons) and Brd2^{BD2 L383}V Val383 (stick, yellow carbons) are highlighted.

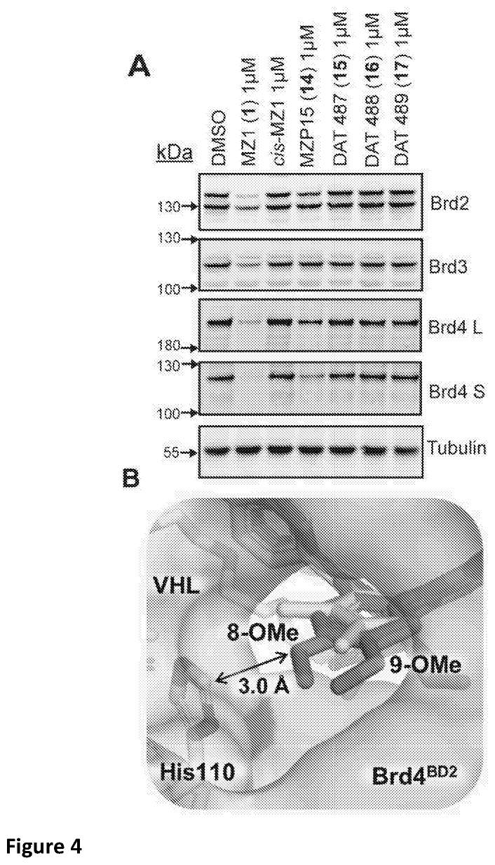

FIG. 4. First generation IBET-762-based B&H-PROTACs are inactive against BromoTag-Brd2 due to proposed steric clash in the MZ1-like ternary complex. (A) Western blot data for BET protein levels after treatment of PROTAC treatment over 6 h in heterozygous BromoTag-Brd2 HEK293 cells. Bands are normalised to tubulin and compared to a vehicle control (DMSO) to derive DC₅₀ values that enable rank order of each PROTAC. (B) Alignment of ternary complex between Brd4^{BD2} (green, surface representation), MZ1 (1, stick, grey carbons) and VHL (cyan, surface representation) (PDB code: 5T35), with ET (6, 8-OMe, stick, pink carbons, 4QEW) and 9-ME-1 (7, 9-OMe, stick, blue carbons, 503C) to show potential clash with VHL by the bulkier 8/9-methoxyphenyl group. His110 is highlighted (stick, cyan carbons).

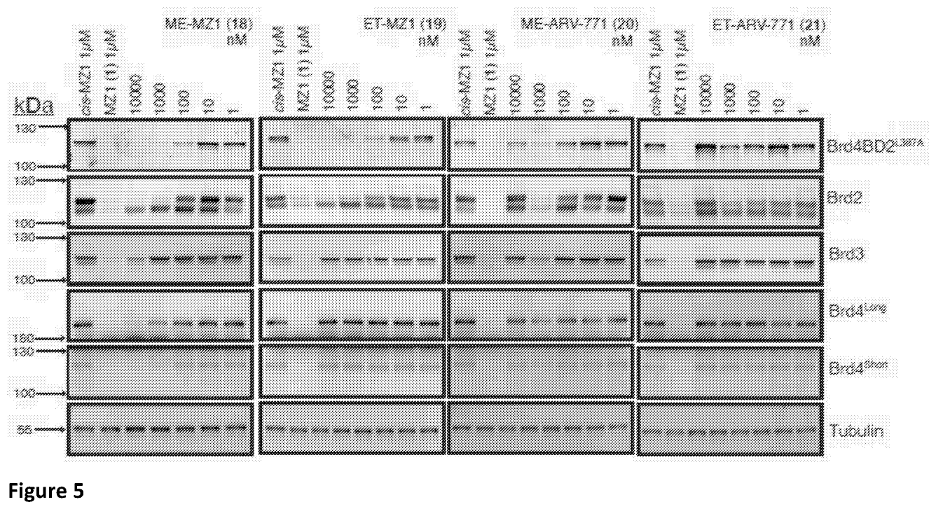

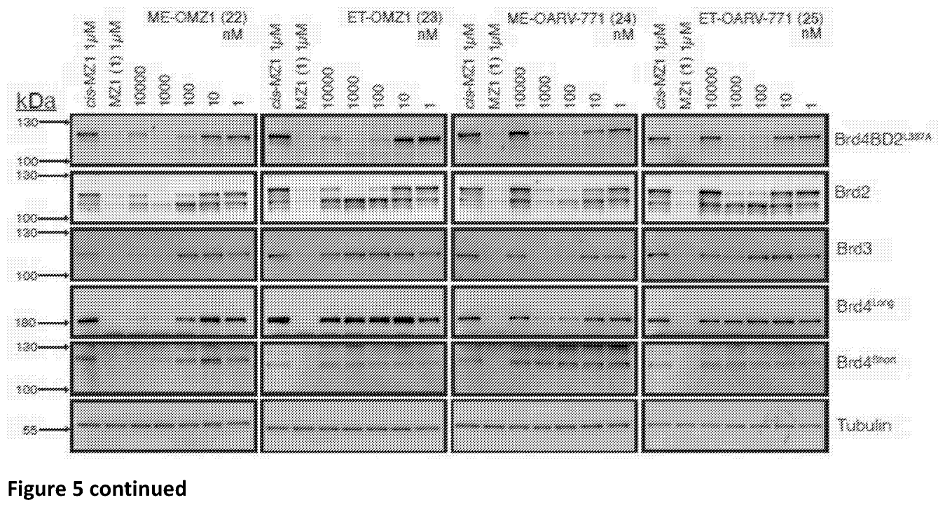

FIG. 5: Biological evaluation of second-generation B&H-PROTACs in BromoTag-Brd2 HEK293 cells. Western blot data for BET protein levels monitored from 10 μM to 1 nM compound treatment over 6 h in heterozygous BromoTag-Brd2 HEK293 cells. Bands are normalised to tubulin and negative control (cis-MZ1) to derive DC₅₀ values that enable rank order of each PROTAC.

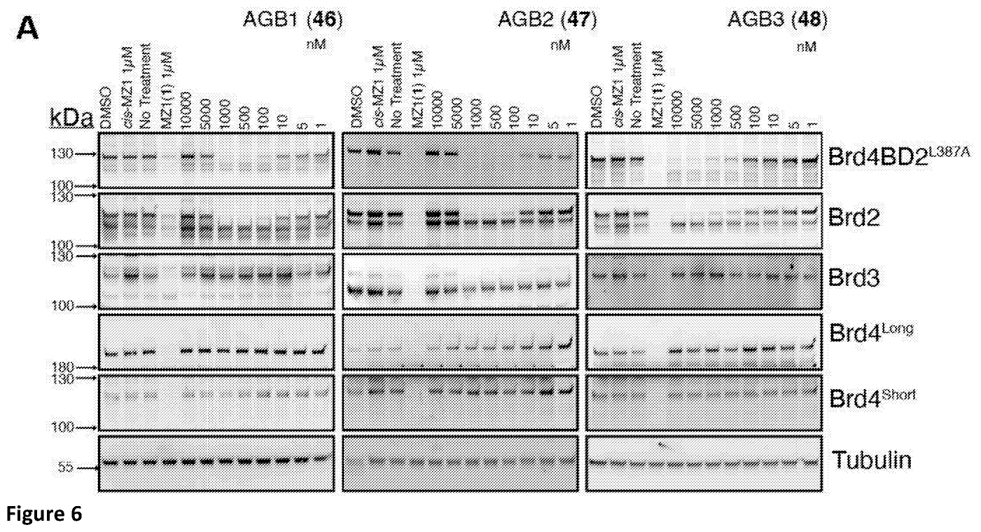

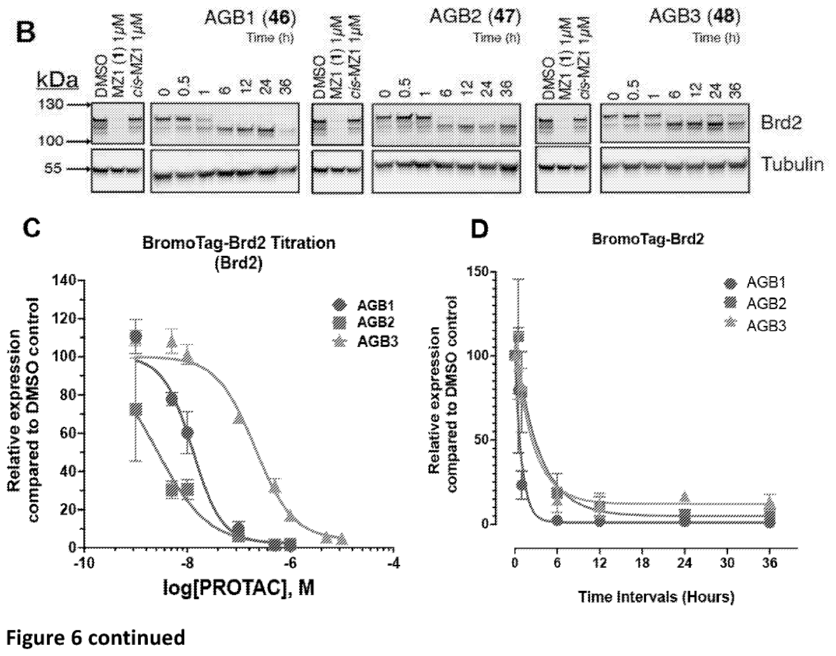

FIG. 6: Biological evaluation of AGB1, AGB2 and AGB3 in BromoTag-Brd2 HEK293 cells. (A) Western blot data for BET protein levels monitored from 10 μM to 1 nM compound treatment over 6 h in heterozygous BromoTag-Brd2 HEK293 cells. (B) Time course western blot data of Brd2 levels in heterozygous BromoTag-Brd2 HEK293 cells upon 500 nM treatment of AGB1 and AGB2 and 1 μM treatment of AGB3 over 36 h. (C & D) Plots to calculate (C) DC₅₀ and (D) t_{1/2} values for compounds enabling determination that AGB1 is the best choice for further validation. Western blots from (A) and (B) were normalised to tubulin and compared to a vehicle control (DMSO) to derive pDC₅₀ or t_{1/2} values that enable rank order of each PROTAC.

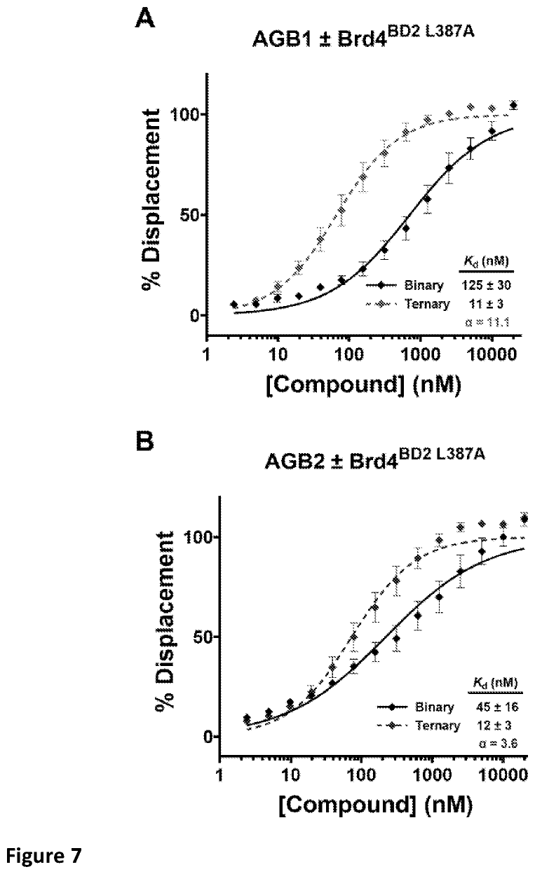

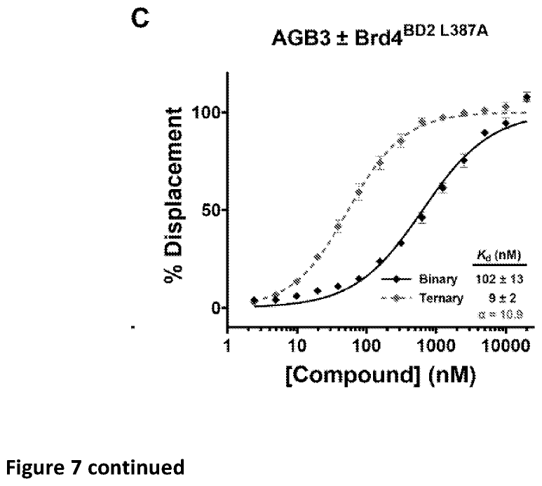

FIG. 7: Fluorescence polarization (FP) of B&H-PROTAC binary and ternary complex binding. Binary and ternary complex formation FP data for 46 (A), 47 (B) and 48 (C) to VHL alone (black solid line) or preincubated with Brd4^{BD2 L387A} to VHL (coloured dashed line) respectively. Error bars and K_{d} values are mean (±S.E.M.) from N=4 for binary and ternary binding to VHL. Left-shift between binary and ternary data indicates positive cooperativity. Cooperativity (a) calculated as a ratio of K_{d}^{binary}/K_{d}^{ternary}.

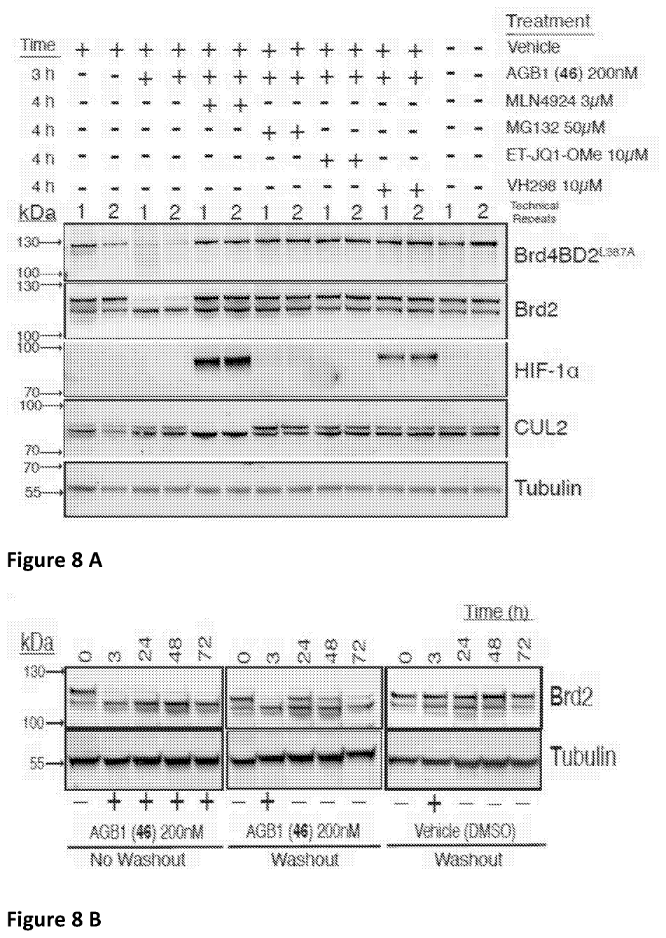

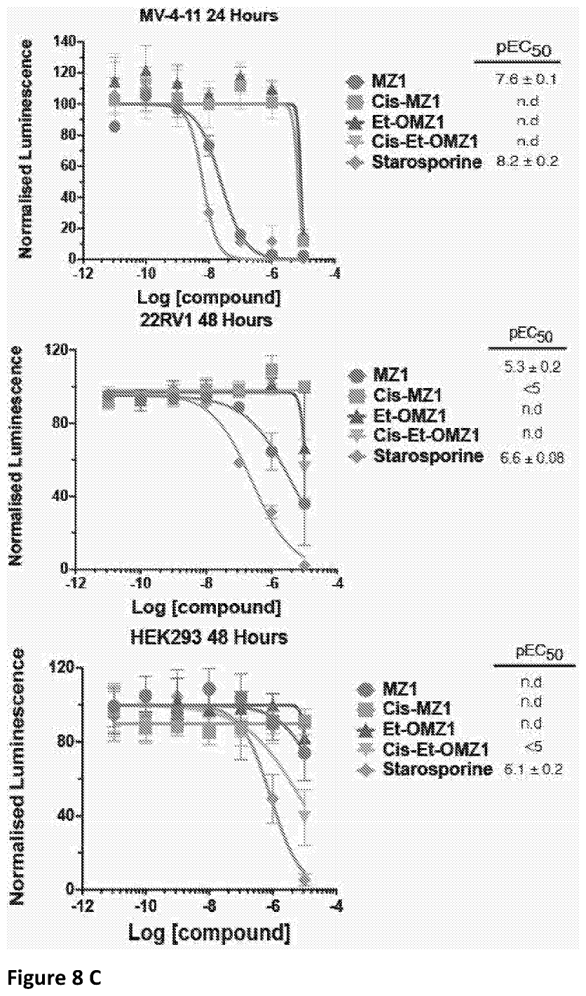

FIG. 8. Cellular mechanistic characterization of AGB1 degradation activity. (A) Western blot illustrating the on-target degradation activity of 46 is dependent on the activity of CRL2^{VHL}, proteasome and on BromoTag target engagement. BromoTag-Brd2 HEK293 cells were treated with 200 nM 46 (3 h) following pre-treatment (1 h) with proteasome inhibitor MG132, neddylation inhibitor MLN4924, VHL inhibitor VH298 or BromoTag inhibitor ET-JQ1-OMe or DMSO vehicle. (B) Western blots demonstrating recovery of BromoTag-Brd2 post removal of 200 nM 46 after a 3 h treatment in heterozygous BromoTag-Brd2 HEK293 cells. Control experiments for no-wash, and vehicle treatments are included. Bands are normalised to tubulin protein levels and compared to a vehicle control (DMSO) to quantify final protein levels of BromoTag-Brd2. (C) Effect on antiproliferation of 46 compared to MZ1 and non-degrader controls 52 and cis-MZ1. Staurosporine was used as positive control for cytotoxicity. MV-4-11, 22Rv1 and HEK293 cells were treated with varying concentrations of compound and after 24 h, 48 h and 48 h respectively, were subjected to Promega CellTiter-Glo cell viability assay. pEC₅₀ values (±S.E.M) are mean from N=2 for MV-4-11 and 22Rv1 cells, and N=3 for HEK293 cells from data normalized from vehicle control (DMSO).

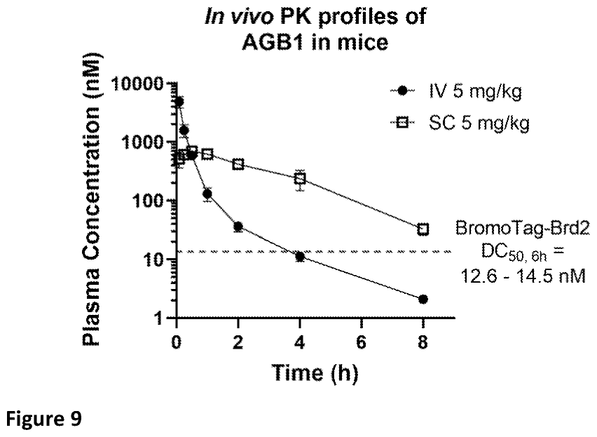

FIG. 9. Plasma Stability and In vivo Pharmacokinetic Studies of AGB1 with Mice. (A) Percentage of AGB1 remaining after 0, 5, 15, 30, 45 and 60 min in mouse plasma at 37° C., normalized to 0 min time point, with two independent repeats per time point. (B) Male C57BL/6 mice were treated with a single 5 mg/kg dose of 46 by either intravenous (IV, black dots) or subcutaneous (SC, hollow squares) injection and 46 blood plasma concentration was measured at seven time points. Data is mean (±S.D.) from three independent repeats at each time point. The red dashed line indicates the DC_{50, 6 h} of 46 for degrading BromoTag-Brd2.

### Experimental Procedures

### Chemistry.

Synthesis. Chemicals, commercially available, were purchased from Apollo Scientific, Sigma-Aldrich, Fluorochem, or Manchester Organics and used without any further purification. All reactions were carried out using anhydrous solvents. Reactions were monitored using either: an Agilent Technologies 1200 series analytical HPLC (High Performance Liquid Chromatography) connected to an Agilent Technologies 6130 quadrupole LC/MS containing an Agilent diode array detector and a Waters XBridge C18 column (50 mm×2.1 mm, 3.5 μm particle size). Samples were eluted with a 3 min gradient of 5% to 95% MeCN:water containing 0.1% formic acid at a flow rate of 0.7 mL/min; or a Shimadzu HPLC/MS 2020 with photodiode array detector and a Hypersil Gold column (1.9 μm 50×2.1 mm). Samples were eluted with a 3 min gradient of of 5% to 95% MeCN:water containing 0.1% formic acid at a flow rate of 0.8 mL/min. Intermediates were purified by flash column chromatography using a Teledyne Isco Combiflash Rf or Rf200i, with Normal Phase RediSep Rf Disposable Columns or with Reverse Phase RediSep Rf Gold C18 Reusable Columns. Final compounds were purified by HPLC using a Gilson Preparative HPLC System equipped with a Waters X-Bridge C18 column (100 mm×19 mm; 5 μm particle size) using a gradient from 5% to 95% of acetonitrile in water containing 0.1% formic acid or ammonia over 10 min at a flow rate of 25 mL/min unless stated otherwise. Compound characterization using NMR was performed either on a Bruker 500 Ultrashield or Bruker Ascend 400 spectrometers. The proton (¹H) and carbon (¹³C) reference solvents used were as follows: d1-Chloroform—CDCl₃ ((δH=7.26 ppm/δC=77.15 ppm), d4-CD₃OD (δH=3.31 ppm/δC=49.00 ppm). Signal patterns are described as singlet (s), doublet (d), triplet (t), quartet (q), quintet (quint.), multiplet (m), broad (br.), or a combination of the listed splitting patterns. Coupling constants (J) are measured in Hertz (Hz). NMR spectra for all compounds were processed using Bruker TopSpin 4.1.1. High resolution mass spectrometry (HRMS) data was performed on a Bruker MicrOTOF II focus ESI Mass Spectrometer connected in parallel to Dionex Ultimate 3000 RSLC system with diode array detector and a Waters XBridge C18 column (50 mm×2.1, 3.5 μm particle size). Samples were eluted with a 6 min gradient of 5% to 95% acetonitrile:water containing 0.1% formic acid at a flow rate of 0.6 mL/min. All compounds are >95% pure by HPLC.

General Procedure A. Azide 9 (synthesised according to Zengerle, M.; Chan, K.-H.; Ciulli, A., ACS Chem Biol. 2015, 10 (8), 1770-1777) (1 eq.) was dissolved in MeOH (125 mL/mmol). A catalytic amount of 10 wt. % Pd/C was added, and the reaction was stirred under an atmosphere of H₂ for 3 h. The reaction mixture was then filtered through PTFE syringe filters and evaporated to dryness to obtain the desired amine quantitative yields. The resulting amine (1 eq.) was added to a solution of acid (1 eq.), HATU (1 eq.), HOAt (1 eq.) and DIPEA (3 eq) in DCM or DMF (2 mL) and left to stir at r.t. for 18 h. This was then purified by HPLC.

General Procedure B. Azides (1 eq.) was dissolved in MeOH (125 mL/mmol). A catalytic amount of 10 wt. % Pd/C was added, and the reaction was stirred under an atmosphere of H₂ for 3 h. The reaction mixture was then filtered through PTFE syringe filters and evaporated to dryness to obtain the desired amines quantitative yields. The resulting amines were added to a solution of alkylated JQ1 acids (1 eq.), COMU (1.5 eq.) and DIPEA (3 eq.) in THF (8 L/mol) and stirred at r.t. for 4 h. The mixtures were then concentrated in vacuo and the residues were purified by HPLC using a linear gradient of 5% to 95% MeCN in 0.1% formic acid in water over 12 min to afford amides as mixtures of two diastereomers.

General Procedure C. Alkylated JQ1 acids (1 eq.), EDC·HCl (2 eq.) were dissolved in THF (15 mL/mmol) and stirred at r.t. for 5 mins. DMAP (3 eq) and alcohols (2 eq.) were then added and the reaction was left to stir at r.t. for 16 h. The mixtures were then concentrated in vacuo and the residues were purified by HPLC using a linear gradient of 5% to 95% MeCN in 0.1% formic acid in water over 12 min to afford amides as mixtures of two diastereomers.

General Procedure D. Compound 29 (120 mg, 0.29 mmol) was dissolved in THF (5.2 mL) and cooled to −78° C. A solution of 0.5 M KHMDS in toluene (812 μL, 0.41 mmol) was added dropwise and the reaction was left to stir at −78° C. for 1 h. Alkyl iodide (0.41 mmol) was then added and the reaction was stirred for a further 10 min at −78° C. before warming to r.t. and leaving to stir for 16 h. The mixture was then concentrated in vacuo and purified by HPLC using a linear gradient of 30% to 70% MeCN in 0.1% formic acid in water over 12 min to afford alkylated JQ1-OMe derivatives.

General Procedure E. (2S,3S) diastereomers (1 eq.) and NaOMe (10 eq.) were dissolved in MeOH (60 L/mol) in a closed, N₂ purged, microwave vial and heated to 120° C. under microwave irradiation for 40 mins. The reaction was the stirred at 60° C. before acidifying with a few drops of AcOH. The reaction was then cooled to r.t. and concentrated in vacuo. The residues were purified by HPLC using a linear gradient of 30% to 70% MeCN in 0.1% formic acid in water over 12 min.

General Procedure F. ET-JQ1-OH (45, synthesised according to Bond, A. G.; Testa, A.; Ciulli, A., Org Biomol Chem. 2020, 18 (38), 7533-7539) (1 eq.) was dissolved in DCM (9 L/mol) under an atmosphere of N₂. Thionyl chloride (15 eq.) was then added and the reaction was left to stir at r.t. for 3 h and conversion to the acid chloride was monitored by LCMS in MeOH (monitor through mass of methyl ester (˜443)). The mixture was evaporated to dryness to afford the acid chloride intermediate quantitatively. Alcohols (1 eq.) were dissolved in DCM (9 L/mol) and added to the acid chloride. This was left to stir at r.t. for 16 h. The mixtures were then concentrated in vacuo and purified.

(2S,4R)-1-((2S)-2-(tert-butyl)-17-(6-(4-chlorophenyl)-8-methoxy-1-methyl-4H-benzo[fj][1,2,4]triazolo[4,3-a][1,4]diazepin-4-yl)-4,16-dioxo-6,9,12-trioxa-3,15-diazaheptadecanoyl)-4-hydroxy-N-(4-(4-methylthiazol-5-yl)benzyl)pyrrolidine-2-carboxamide (MZP-15) (14). Following general procedure A, compound 14 was obtained using acid 10 (synthesised according to Chung, C.-W et al. J Med Chem. 2011, 54 (11), 3827-3838) without HOAt, in DCM and was purified by HPLC using a linear gradient of 5% to 95% MeCN in 0.1% formic acid in water over 12 min to afford 14 as a mixture of two diastereomers. Yield: 16.5 mg (47%); ¹H NMR (400 MHz, CDCl₃): δ=8.70 (s, 1H), 8.63-8.61 (m, 1H), 8.31-8.28 (m, 1H), 8.10-8.03 (m, 1H), 7.52-7.45 (m, 3H), 7.41-7.28 (m, 8H), 7.20-7.16 (m, 1H), 6.86-6.84 (m, 1H), 4.87-4.81 (m, 1H), 4.71-4.63 (m, 2H), 4.57-4.48 (m, 2H), 4.41-4.28 (m, 2H), 4.22-4.06 (m, 3H), 3.78 (s, 3H), 2.57-2.54 (m, 3H), 2.51-2.49 (m, 3H), 2.46-2.35 (m, 1H), 2.27-2.20 (m, 1H), 1.01-0.99 ppm (m, 9H); ¹³C NMR (101 MHz, CDCl₃): δ=171.7, 171.5, 171.3, 171.1, 171.04, 171.01, 170.9, 170.7, 166.6, 166.4, 158.3, 156.74, 156.70, 150.6, 150.3, 148.3, 138.7, 138.6, 137.2, 137.1, 137.0, 131.1, 131.0, 130.62, 130.55, 130.4, 130.3, 129.51, 129.47, 129.2, 128.7, 128.6, 128.2, 128.1, 126.2, 124.95, 124.91, 120.3, 118.2, 118.1, 116.0, 71.7, 71.2, 70.9, 70.8, 70.6, 70.5, 70.4, 70.3, 70.22, 70.15, 59.2, 59.1, 57.5, 57.3, 56.9, 56.0, 53.6, 53.5, 43.2, 40.0, 39.9, 38.1, 38.0, 36.9, 36.7, 35.8, 35.6, 26.6, 16.0, 12.11, 12.07; HRMS m/z calc. for C₅₀H₆₁ClN₉O₉S [M+H]⁺ 998.3996, found 998.3996.

(2S,4R)-1-((2S,17R*)-2-(tert-butyl)-17-((S*)-6-(4-chlorophenyl)-8-methoxy-1-methyl-4H-benzo[fj][1,2,4]triazolo[4,3-a][1,4]diazepin-4-yl)-4,16-dioxo-6,9,12-trioxa-3,15-diazanonadecanoyl)-4-hydroxy-N-(4-(4-methylthiazol-5-yl)benzyl)pyrrolidine-2-carboxamide (DAT487) (15). Following general procedure A, compound 15 was obtained using acid 11 (synthesised according to Runcie, A. C. et al. Chem Sci. 2018, 9 (9), 2452-2468) in DMF and was purified by HPLC using a linear gradient of 5% to 95% MeCN in 0.1% ammonia in water over 12 min to afford 15 as a mixture of two diastereomers. Yield: 8.3 mg (23%); ¹H NMR (400 MHz, MeOD): δ=8.86 (d, J=1.3 Hz, 1H), 7.72-7.67 (m, 1H), 7.56 (d, J=8.5 Hz, 2H), 7.46-7.34 (m, 7H), 6.93-6.91 (m, 1H), 4.79-4.32 (m, 5H), 4.29-4.25 (m, 1H), 4.11-4.02 (m, 2H), 3.90-3.85 (m, 1H), 3.82-3.77 (m, 4H), 3.74-3.40 (m, 13H), 3.30-3.14 (m, 1H), 2.59-2.58 (m, 3H), 2.47-2.46 (m, 3H), 2.27-2.17 (m, 2H), 2.11-2.03 (m, 1H), 1.75-1.63 (m, 1H), 1.07-1.01 ppm (m, 12H); ¹³C NMR (101 MHz, MeOD): δ=175.9, 174.42, 174.39, 172.1, 171.6, 168.8, 168.7, 159.9, 157.3, 152.8, 152.6, 149.0, 140.3, 140.2, 138.6, 138.14, 138.11, 133.4, 132.1, 131.5, 131.3, 130.3, 129.6, 129.0, 128.9, 127.5, 126.8, 119.2, 116.8, 72.3, 72.2, 71.75, 71.67, 71.5, 71.4, 71.0, 70.5, 70.4, 60.8, 58.5, 58.4, 58.1, 56.4, 49.84, 49.76, 43.8, 43.7, 40.4, 39.0, 37.2, 37.1, 26.99, 26.96, 24.53, 24.46, 15.9, 11.7, 11.6; LCMS m/z calc. for C₅₂H₆₆ClN₉O₉S [M+2H]²⁺ 513.7, found 514.1.

(2S,4R)-1-((2S,17R*)-2-(tert-butyl)-17-((S*)-6-(4-chlorophenyl)-9-methoxy-1-methyl-4H-benzo[fj][1,2,4]triazolo[4,3-a][1,4]diazepin-4-yl)-4,16-dioxo-6,9,12-trioxa-3,15-diazaoctadecanoyl)-4-hydroxy-N-(4-(4-methylthiazol-5-yl)benzyl)pyrrolidine-2-carboxamide (DAT488) (16). Following general procedure A, compound 16 was obtained using acid 12 (synthesised according to Runcie, A. C. et al., 2018 (supra)), in DMF and was purified by HPLC using a linear gradient of 5% to 95% MeCN in 0.1% ammonia in water over 12 min to afford 16 as a mixture of two diastereomers. Yield: 13.8 mg (28%); ¹H NMR (400 MHz, MeOD): δ=8.87-8.86 (m, 1H), 7.49-7.34 (m, 9H), 7.31-7.29 (m, 1H), 7.16-7.11 (m, 1H), 4.71-4.69 (m, 1H), 4.62-4.56 (m, 1H), 4.55-4.46 (m, 2H), 4.38-4.32 (m, 1H), 4.25-4.21 (m, 1H), 4.09-4.00 (m, 2H), 3.97-3.96 (m, 3H), 3.90-3.72 (m, 3H), 3.71-3.59 (m, 11H), 3.53-3.42 (m, 2H), 2.66-2.64 (m, 3H), 2.47-2.45 (m, 3H), 2.25-2.18 (m, 1H), 2.11-2.04 (m, 1H), 1.36-1.32 (m, 3H), 1.05-1.02 ppm (m, 9H); ¹³C NMR (101 MHz, MeOD): δ=177.4, 174.39, 174.36, 172.1, 171.7, 171.6, 168.1, 168.0, 163.6, 156.8, 152.9, 152.8, 149.0, 140.3, 140.2, 138.9, 137.9, 135.8, 134.34, 134.32, 133.4, 132.4, 131.5, 130.4, 129.4, 128.9, 122.5, 115.2, 110.6, 72.2, 71.7, 71.6, 71.4, 71.1, 71.0, 70.7, 60.8, 60.7, 58.1, 56.7, 43.9, 43.7, 40.5, 38.9, 37.1, 27.0, 20.0, 16.1, 16.0, 15.9, 11.9; LCMS m/z calc. for C₅₁H₆₄ClN₉O₉S [M+2H]²⁺ 506.7, found 507.1.

(2S,4R)-1-((2S,17R*)-2-(tert-butyl)-17-((S*)-6-(4-chlorophenyl)-9-methoxy-1-methyl-4H-benzo[f][1,2,4]triazolo[4,3-a][1,4]diazepin-4-yl)-4,16-dioxo-6,9,12-trioxa-3,15-diazanonadecanoyl)-4-hydroxy-N-(4-(4-methylthiazol-5-yl)benzyl)pyrrolidine-2-carboxamide (DAT489 17). Following general procedure A, compound 17 was obtained using acid 13 (synthesised according to Runcie, A. C. et al., 2018 (supra)), in DMF and was purified by HPLC using a linear gradient of 5% to 95% MeCN in 0.1% ammonia in water over 12 min to afford 17 as a mixture of two diastereomers. Yield: 4.8 mg (20%); ¹H NMR (400 MHz, MeOD): δ=8.87-8.86 (m, 1H), 7.54-7.50 (m, 2H), 7.46-7.37 (m, 7H), 7.26 (d, J=2.5 Hz, 1H), 7.15-7.12 (m, 1H), 4.74-4.70 (m, 1H), 4.69-4.51 (m, 2H), 4.50-4.46 (m, 1H), 4.42-4.32 (m, 1H), 4.29-4.25 (m, 1H), 4.11-4.02 (m, 2H), 3.96 (s, 3H), 3.89-3.78 (m, 2H), 3.74-3.39 (m, 13H), 3.29-3.14 (m, 1H), 2.63-2.62 (m, 3H), 2.47-2.46 (m, 3H), 2.28-2.18 (m, 2H), 2.11-2.02 (m, 1H), 1.75-1.62 (m, 1H), 1.06-1.01 ppm (m, 12H); ¹³C NMR (101 MHz, MeOD): δ=176.0, 174.42, 174.39, 172.1, 171.6, 169.3, 169.2, 163.71, 163.69, 157.27, 157.26, 152.8, 152.6, 149.0, 140.3, 140.2, 139.1, 138.0, 138.0, 136.0, 134.30, 134.28, 133.4, 132.3, 131.5, 130.3, 129.5, 129.02, 128.95, 122.6, 115.1, 110.6, 72.4, 72.2, 71.75, 71.68, 71.50, 71.48, 71.4, 71.09, 71.06, 70.5, 70.4, 60.80, 60.78, 58.4, 58.3, 58.14, 58.10, 56.7, 49.8, 49.7, 43.8, 43.7, 40.4, 39.0, 37.2, 37.1, 27.00, 26.96, 24.5, 24.4, 15.9, 11.9, 11.7, 11.6; LCMS m/z calc. for C₅₂H₆₆ClN₉O₉S [M+2H]²⁺ 513.7, found 514.2.

(2S,4R)-1-((2S,17R*)-2-(tert-butyl)-17-((S*)-4-(4-chlorophenyl)-2,3,9-trimethyl-6H-thieno[3,2-f][1,2,4]triazolo[4,3-a][1,4]diazepin-6-yl)-4,16-dioxo-6,9,12-trioxa-3,15-diazaoctadecanoyl)-4-hydroxy-N-(4-(4-methylthiazol-5-yl)benzyl)pyrrolidine-2-carboxamide (ME-MZ1) (18). Following general procedure B, compound 18 was obtained using azide 9 (synthesised according to Chung, C.-W et al., 2011 (supra)) and alkylated JQ1 acid 32 to afford 18 as a mixture of two diastereomers. Yield: 1.6 mg (19%); ¹H NMR (400 MHz, CDCl₃): δ=8.67 (s, 1H), 8.07 (t, J=5.4 Hz, 1H), 7.89 (t, J=5.1 Hz, 1H), 7.68 (t, J=6.0 Hz, 1H), 7.46-7.24 (m, 9H), 4.85-4.79 (m, 1H), 4.77-4.63 (m, 1H), 4.63-4.47 (m, 2H), 4.42-4.36 (m, 1H), 4.31-4.23 (m, 2H), 4.18-4.01 (m, 3H), 3.96-3.85 (m, 1H), 3.81-3.36 (m, 16H), 2.65-2.60 (m, 3H), 2.51 (s, 3H), 2.50-2.37 (m, 4H), 2.26-2.11 (m, 1H), 1.73-1.64 (m, 3H), 1.42-1.35 (m, 3H), 1.01-0.94 ppm (m, 9H); ¹³C NMR (101 MHz, CDCl₃): δ=175.2, 171.51, 171.49, 171.2, 170.7, 170.5, 163.3, 163.2, 158.2, 155.14, 155.09, 150.4, 149.8, 148.6, 138.6, 138.5, 136.9, 136.8, 136.69, 136.65, 131.9, 131.25, 131.15, 130.8, 130.2, 129.6, 129.5, 128.9, 128.8, 128.2, 128.0, 71.6, 71.4, 70.9, 70.6, 70.5, 70.4, 70.3, 70.25, 70.20, 70.1, 60.5, 60.1, 59.1, 58.9, 57.5, 57.3, 56.8, 56.7, 43.3, 43.2, 42.7, 42.6, 39.9, 36.4, 36.3, 35.9, 35.8, 26.6, 26.5, 16.4, 16.2, 16.1, 14.6, 13.3, 11.91, 11.87; H RMS m/z calc. for C₅₀H₆₃ClN₉O₈S₂ [M+H]⁺ 1016.3924, found: 1016.3905.

(2S,4R)-1-((2S,17R*)-(tert-butyl)-17-((S*)-4-(4-chlorophenyl)-2,3,9-trimethyl-6H-thieno[3,2-f][1,2,4]triazolo[4,3-a][1,4]diazepin-6-yl)-4,16-dioxo-6,9,12-trioxa-3,15-diazanonadecanoyl)-4-hydroxy-N-(4-(4-methylthiazol-5-yl)benzyl)pyrrolidine-2-carboxamide (ET-MZ1) (19). Following general procedure B, compound 19 was obtained using azide 9 (synthesised according to Chung, C.-W et al., 2011 (supra)) and alkylated JQ1 acid 33 to afford 19 as a mixture of two diastereomers. Yield: 5.3 mg (47%); ¹H NMR (500 MHz, CDCl₃): δ=8.68-8.66 (m, 1H), 8.10-8.05 (m, 1H), 7.83-7.79 (m, 1H), 7.69-7.65 (m, 1H), 7.40-7.26 (m, 8H), 7.21-7.16 (m, 2H), 4.87-4.76 (m, 2H), 4.68-4.49 (m, 2H), 4.49-4.36 (m, 1H), 4.28-4.24 (m, 1H), 4.18-4.00 (m, 3H), 3.81-3.74 (m, 1H), 3.74-3.34 (m, 13H), 2.65-2.62 (m, 3H), 2.53-2.51 (m, 3H), 2.49-2.44 (m, 1H), 2.41-2.38 (m, 3H), 2.36-2.30 (m, 1H), 2.25-2.11 (m, 1H), 1.97-1.89 (m, 1H), 1.71-1.59 (m, 4H), 1.03-0.93 ppm (m, 12H); ¹³C NMR (126 MHz, CDCl₃): δ=174.2, 173.9, 171.7, 171.6, 171.2, 170.4, 170.3, 163.8, 163.2, 155.3, 155.1, 150.3, 149.9, 149.8, 148.6, 138.7, 138.6, 136.95, 136.91, 136.8, 136.7, 132.1, 131.94, 131.90, 131.3, 131.1, 131.0, 130.9, 130.8, 130.7, 130.2, 130.1, 129.9, 129.6, 129.5, 129.0, 128.8, 128.3, 127.6, 71.3, 71.2, 71.1, 70.9, 70.8, 70.7, 70.50, 70.47, 70.3, 70.18, 70.15, 59.8, 59.4, 59.2, 58.9, 57.63, 57.59, 56.8, 50.3, 50.1, 43.3, 43.0, 39.9, 39.8, 36.9, 36.4, 35.90, 35.85, 26.6, 23.7, 23.2, 16.21, 16.19, 14.6, 14.5, 13.2, 12.0; HRMS m/z calc. for C₅₁H₆₅ClN₉O₈S₂ [M+H]⁺ 1030.4081, found: 1030.3943.

(2S,4R)-1-((2S,15R*)-(tert-butyl)-15-((S*)-4-(4-chlorophenyl)-2,3,9-trimethyl-6H-thieno[3,2-f][1,2,4]triazolo[4,3-a][1,4]diazepin-6-yl)-4,14-dioxo-6,10-dioxa-3,13-diazahexadecanoyl)-4-hydroxy-N—((S)-1-(4-(4-methylthiazol-5-yl)phenyl)ethyl)pyrrolidine-2-carboxamide (ME-ARV-771) (20). Following general procedure B, compound 20 was obtained using azide 42 (synthesised according to Klein, V. et al. ChemRxiv 2021) and alkylated JQ1 acid 32 to afford 20 as a mixture of two diastereomers. Yield: 5.2 mg (31%); ¹H NMR (500 MHz, CDCl₃): δ=8.71-8.69 (m, 1H), 7.66-7.61 (m, 1H), 7.57-7.21 (m, 9H), 5.14-4.98 (m, 1H), 4.86-4.80 (m, 1H), 4.65-4.47 (m, 2H), 4.34-4.24 (m, 1H), 4.21-3.77 (m, 4H), 3.75-3.33 (m, 11H), 2.69-2.63 (m, 3H), 2.53-2.50 (m, 3H), 2.41 (s, 3H), 2.38-2.28 (m, 1H), 2.23-2.10 (m, 1H), 1.92-1.71 (m, 2H), 1.67 (d, J=3.1 Hz, 3H), 1.48 (d, J=21.4 Hz, 3H), 1.43-1.35 (m, 3H), 1.10-1.05 ppm (m, 9H); ¹³C NMR (126 MHz, CDCl₃): δ=175.3, 175.0, 171.5, 171.4, 170.8, 170.6, 170.42, 170.38, 164.1, 163.9, 155.0, 154.9, 150.5, 150.22, 150.16, 148.2, 143.9, 143.7, 137.3, 136.4, 136.1, 132.0, 131.8, 131.33, 131.26, 130.6, 130.5, 130.3, 129.6, 129.55, 129.48, 128.92, 128.88, 126.6, 70.4, 70.32, 70.29, 70.2, 69.6, 69.4, 69.2, 67.84, 67.77, 59.9, 59.8, 58.9, 58.8, 57.5, 57.3, 57.1, 57.0, 49.1, 48.9, 42.6, 42.3, 39.7, 39.5, 36.6, 36.5, 35.5, 35.3, 29.53, 29.46, 26.7, 22.4, 22.1, 16.5, 16.3, 16.1, 14.6, 13.3, 11.8, 11.7; HRMS m/z calc. for C₅₀H₆₃ClN₉O₇S₂ [M+H]⁺ 1000.3975, found: 1000.3975.

(2S,4R)-1-((2S,15R*)-(tert-butyl)-15-((S*)-4-(4-chlorophenyl)-2,3,9-trimethyl-6H-thieno[3,2-f][1,2,4]triazolo[4,3-a][1,4]diazepin-6-yl)-4,14-dioxo-6,10-dioxa-3,13-diazaheptadecanoyl)-4-hydroxy-N—((S)-1-(4-(4-methylthiazol-5-yl)phenyl)ethyl)pyrrolidine-2-carboxamide (ET-ARV-771) (21). Following general procedure B, compound 21 was obtained using azide 42 (synthesised according to Klein, V. et al. 2021(supra)) and alkylated JQ1 acid 33 to afford 21 as a mixture of two diastereomers. Yield: 3.4 mg (27%); ¹H NMR (400 MHz, CDCl₃): δ=8.68 (s, 1H), 7.94-7.90 (m, 1H), 7.71-7.65 (m, 1H), 7.44-7.31 (m, 7H), 7.25-7.22 (m, 1H), 7.17-7.13 (m, 1H), 5.14-4.87 (m, 1H), 4.75-4.58 (m, 1H), 4.53-4.44 (m, 1H), 4.09-3.98 (m, 1H), 3.94-3.87 (m, 2H), 3.80-3.31 (m, 11H), 2.67-2.61 (m, 2H), 2.54-2.49 (m, 3H), 2.41 (s, 2H), 2.37-2.14 (m, 2H), 1.98-1.47 (m, 11H), 1.11-0.94 ppm (m, 12H); ¹³C NMR (101 MHz, CDCl₃): δ=174.04, 174.00, 172.3, 171.5, 170.59, 170.55, 170.32, 170.25, 163.6, 163.2, 162.4, 155.3, 155.1, 150.4, 144.0, 143.4, 137.1, 136.9, 136.6, 131.3, 131.2, 131.1, 130.64, 130.59, 130.5, 130.10, 130.07, 130.0, 129.6, 129.4, 128.84, 128.80, 128.7, 126.6, 126.5, 70.4, 70.30, 70.26, 70.2, 69.6, 69.4, 69.1, 68.1, 67.7, 59.6, 59.0, 58.8, 58.6, 57.4, 57.1, 57.0, 50.3, 50.2, 49.1, 48.8, 39.8, 39.4, 36.6, 35.7, 35.6, 29.8, 29.6, 29.5, 26.7, 23.9, 23.4, 22.5, 21.9, 16.2, 14.61, 14.56, 13.3, 12.00, 11.97, 11.91, 11.87; HRMS m/z calc. for C₅₁H₆₅ClN₉O₇S₂ [M+H]⁺ 1014.4131, found: 1014.4126.

(S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl (R*)-2-((S*)-4-(4-chlorophenyl)-2,3,9-trimethyl-6H-thieno[3,2-f][1,2,4]triazolo[4,3-a][1,4]diazepin-6-yl)propanoate (ME-OMZ1) (22). Following general procedure C, compound 22 was obtained using alkylated JQ1 acid 32 and alcohol 41 (synthesised according to Klein, V. et al. 2021(supra)) to afford 22 as a mixture of two diastereomers. Yield: 1.1 mg (17%); ¹H NMR (400 MHz, CDCl₃): δ=8.69-8.67 (m, 1H), 7.67-7.65 (m, 1H), 7.44-7.28 (m, 10H), 4.78-4.73 (m, 1H), 4.69-4.50 (m, 3H), 4.41-4.24 (m, 3H), 4.13-3.81 (m, 5H), 3.77-3.49 (m, 12H), 2.63 (d, J=15.9 Hz, 3H), 2.59-2.50 (m, 4H), 2.42 (s, 3H), 1.73-1.66 (m, 3H), 1.58-1.48 (m, 3H), 0.99-0.94 ppm (m, 9H); ¹³C NMR (101 MHz, CDCl₃): δ=175.4, 171.6, 170.9, 170.6, 170.5, 163.2, 154.5, 150.4, 149.8, 148.7, 142.1, 140.3, 138.4, 136.9, 136.6, 132.4, 131.7, 131.1, 131.0, 131.0, 130.0, 129.6, 128.9, 128.8, 128.3, 71.30, 71.28, 71.0, 70.93, 70.89, 70.7, 70.6, 70.5, 70.44, 70.36, 69.3, 64.1, 64.0, 61.8, 61.7, 60.2, 60.1, 58.7, 58.6, 57.3, 56.8, 56.7, 43.4, 42.7, 42.6, 36.1, 36.0, 35.2, 26.6, 16.2, 16.1, 15.4, 15.3, 14.60, 14.56, 13.3, 11.9; HRMS m/z calc. for C₅₀H₆₂ClN₈O₉S₂ [M+H]⁺ 1017.3764, found: 1017.3780.

(S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl (R*)-2-((S*)-4-(4-chlorophenyl)-2,3,9-trimethyl-6H-thieno[3,2-f][1,2,4]triazolo[4,3-a][1,4]diazepin-6-yl)butanoate (ET-OMZ1) (23). Following general procedure C, compound 23 was obtained using alkylated JQ1 acid 33 and alcohol 41 (synthesised according to Klein, V. et al. 2021(supra)) to afford 23 as a mixture of two diastereomers. Yield: 0.7 mg (10%); ¹H NMR (500 MHz, CDCl₃): δ=8.67 (s, 1H), 7.41-7.23 (m, 10H), 4.77 (t, J=7.9 Hz, 1H), 4.60-4.31 (m, 6H), 4.24 (d, J=10.9 Hz, 1H), 4.15-4.11 (m, 1H), 4.05-3.89 (m, 3H), 3.80-3.74 (m, 2H), 3.72-3.52 (m, 10H), 2.65 (s, 3H), 2.60-2.51 (m, 4H), 2.41 (s, 3H), 2.17-2.10 (m, 2H), 1.71-1.64 (m, 4H), 1.05-0.93 ppm (m, 12H); ¹³C NMR (126 MHz, CDCl₃): δ=174.8, 174.1, 171.5, 171.1, 170.5, 163.2, 163.0, 154.6, 150.4, 149.9, 148.7, 141.2, 136.9, 136.7, 132.2, 131.8, 131.0, 130.7, 130.0, 129.7, 128.8, 128.4, 128.3, 71.32, 71.30, 70.98, 70.96, 70.91, 70.85, 70.8, 70.7, 70.6, 70.54, 70.51, 70.3, 69.3, 63.9, 63.8, 59.4, 58.5, 57.3, 56.8, 49.8, 49.7, 43.44, 43.41, 36.0, 35.1, 26.6, 23.4, 16.2, 14.6, 13.3, 11.9, 11.7, 16.1, 15.4, 15.3, 14.60, 14.56, 13.3, 11.9; HRMS m/z calc. for C₅₁H₆₄ClN₈O₉S₂ [M+H]⁺ 1031.3921, found: 1031.4061.

2-(3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)propoxy)ethyl (R*)-2-((S*)-4-(4-chlorophenyl)-2,3,9-trimethyl-6H-thieno[3,2-f][1,2,4]triazolo[4,3-a][1,4]diazepin-6-yl)propanoate (ME-OARV-771) (24). Following general procedure C, compound 24 was obtained using alkylated JQ1 acid 32 and alcohol 44 (synthesised according to Klein, V. et al. 2021(supra)) to afford 24 as a mixture of two diastereomers. Yield: 1.8 mg (25%); ¹H NMR (500 MHz, CDCl₃): δ=8.68-8.66 (m, 1H), 7.51-7.44 (m, 1H), 7.42-7.28 (m, 8H), 7.23-7.16 (m, 1H), 5.52 (br. s, 1H), 5.14-5.06 (m, 1H), 4.81-4.71 (m, 1H), 4.70-4.51 (m, 2H), 4.43-4.31 (m, 2H), 4.27 (d, J=10.5 Hz, 1H), 4.13-4.08 (m, 1H), 4.07-3.81 (m, 4H), 3.75-3.54 (m, 8H), 3.19 (br. s, 1H), 2.66-2.62 (m, 3H), 2.59-2.52 (m, 4H), 2.42 (s, 3H), 2.17-2.08 (m, 1H), 1.96-1.85 (m, 2H), 1.69 (d, J=4.8 Hz, 3H), 1.56-1.47 (m, 6H), 1.08-1.05 ppm (m, 9H); ¹³C NMR (126 MHz, CDCl₃): δ=175.4, 174.3, 171.8, 171.5, 170.3, 169.8, 169.7, 169.5, 163.1, 154.5, 150.3, 149.8, 148.7, 136.9, 136.7, 132.2, 131.8, 131.0, 130.7, 130.0, 129.7, 128.9, 128.8, 126.65, 126.60, 73.2, 72.2, 70.5, 70.4, 70.3, 69.3, 69.0, 68.9, 68.0, 67.9, 63.9, 61.9, 60.1, 59.6, 58.8, 58.5, 57.2, 56.8, 56.6, 53.6, 49.1, 49.0, 42.6, 35.6, 35.3, 33.5, 30.0, 26.7, 26.6, 22.4, 22.3, 16.2, 15.3, 14.60, 14.55, 14.3, 13.2, 11.9; HRMS m/z calc. for C₅₀H₆₂ClN₈O₈S₂ [M+H]⁺ 1001.3815, found: 1001.3967.

2-(3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)propoxy)ethyl (R*)-2-((S*)-4-(4-chlorophenyl)-2,3,9-trimethyl-6H-thieno[3,2-f][1,2,4]triazolo[4,3-a][1,4]diazepin-6-yl)butanoate (ET-OARV-771) (25). Following general procedure C, compound 24 was obtained using alkylated JQ1 acid 32 and alcohol 44 (synthesised according to Klein, V. et al. 2021(supra)) to afford 24 as a mixture of two diastereomers. Yield: 1.7 mg (24%); ¹H NMR (500 MHz, CDCl₃): δ=8.66 (s, 1H), 7.47-7.28 (m, 10H), 7.20-7.13 (m, 1H), 5.12-5.04 (m, 1H), 4.81-4.75 (m, 1H), 4.70-4.52 (m, 2H), 4.49-4.31 (m, 2H), 4.28-4.22 (m, 1H), 4.18-4.11 (m, 1H), 4.07-3.94 (m, 2H), 3.91-3.81 (m, 2H), 3.80-3.64 (m, 3H), 3.63-3.54 (m, 5H), 3.07 (s, 1H), 2.67-2.65 (m, 3H), 2.59-2.52 (m, 4H), 2.41 (s, 3H), 2.19-2.05 (m, 2H), 1.96-1.85 (m, 2H), 1.72-1.64 (m, 4H), 1.49-1.46 (m, 3H), 1.07-1.01 ppm (m, 12H); ¹³C NMR (126 MHz, CDCl₃): δ=175.0, 174.9, 171.9, 171.8, 170.42, 170.38, 169.8, 169.7, 163.2, 154.6, 154.5, 150.3, 149.89, 149.85, 148.7, 143.42, 143.36, 136.92, 136.90, 136.71, 136.67, 131.8, 131.0, 130.1, 130.0, 129.7, 128.8, 126.6, 72.2, 70.32, 70.26, 70.2, 69.3, 69.0, 68.9, 68.0, 67.9, 63.8, 62.0, 59.4, 58.45, 58.40, 57.2, 56.8, 56.7, 49.8, 49.7, 49.0, 35.6, 35.4, 35.2, 35.1, 30.1, 26.7, 26.6, 23.43, 23.39, 22.4, 16.2, 14.6, 13.3, 12.0, 11.7; HRMS m/z calc. for C₅₁H₆₄ClN₈O₈S₂ [M+H]⁺ 1015.3972, found: 1015.4032. methyl 2-(5-(4-chlorophenyl)-6,7-dimethyl-2-oxo-2,3-dihydro-1H-thieno[2,3-e][1,4]diazepin-3-yl)acetate (28). Fmoc-Asp(OMe)-OH (26) (1.92 g, 5.19 mmol) was dissolved in DCM (25 mL). Thionyl chloride (3.76 mL, 51.9 mmol) was added and the reaction was left to reflux for 2 h. The reaction mixture was then concentrated in vacuo to yield the intermediate acid chloride. The acid chloride (2.01 g, 5.19 mmol) was dissolved in chloroform (10 mL). (2-amino-4,5-dimethylthiophen-3-yl)(4-chlorophenyl)methanone (27) (1.38 g, 5.19 mmol) was then added and the flask was heated to reflux and stirred for 1 h. The mixture was then cooled to r.t. before TEA (2.89 mL, 20.76 mmol) was added. The flask was heated to reflux for a further 16 h. The reaction mixture was then concentrated in vacuo and redissolved in 1,2-DCE (50 mL) and acidified with AcOH (3.5 mL). This was left to stir at 80° C. for 1 h. The mixture was then evaporated to dryness before redissolving in DCM (50 mL) and washing with 1.0 M HCl solution (40 mL). The aqueous phase was extracted with DCM (3×50 mL) and the combined organic layers were dried with MgSO₄, filtered, and concentrated in vacuo. The residue was purified by flash column chromatography (24 g silica column) using a linear gradient from 0% to 80% EtOAc in heptane to afford 28. Yield: 1.06 g (54%); ¹H NMR (500 MHz, CDCl₃): δ=7.43 (d, J=8.6 Hz, 2H), 7.34 (d, J=8.7 Hz, 2H), 4.26 (dd, J=6.6, 7.4 Hz, 1H), 3.74 (s, 3H), 3.44 (dd, J=7.5, 16.8 Hz, 1H), 3.17 (dd, J=6.5, 16.8 Hz, 1H), 2.29 (s, 3H), 1.60 ppm (s, 3H); LCMS m/z calc. for C₁₈H₁₈ClN₂O₃S [M+H]⁺ 377.1, found: 377.0.

methyl 2-(4-(4-chlorophenyl)-2,3,9-trimethyl-6H-thieno[3,2-f][1,2,4]triazolo[4,3-a][1,4]diazepin-6-yl)acetate ((±)-JQ1-OMe) (29). Compound 28 (344 mg, 0.91 mmol), was dissolved in THF (7 mL) and cooled to −78° C. before addition of a solution of 1.0 M KO^{t}Bu in THF (1.37 mL, 1.37 mmol) and stirred for 30 min. Diethyl chlorophosphate (198 μL, 1.37 mmol) was then added and the reaction was warmed to −10° C. and stirred for 45 min. Acetyl hydrazine (135 mg, 1.82 μmol) was then added and the reaction was left to stir at r.t. for 1 h. n-BuOH (7.8 mL) was then added before heating to 90° C. for 1 h. The reaction was concentrated in vacuo and the residue was purified by flash column chromatography (40 g silica column) using a linear gradient from 30% to 50% EtOAc in heptane to remove starting material and flushing the column with 20% MeOH in DCM. Some fractions were further purified by HPLC using a linear gradient of 35% to 55% MeCN in 0.1% formic acid in water over 12 min to afford 29. Yield: 173 mg (46%); ¹H NMR (400 MHz, CDCl₃): δ=7.41 (d, J=8.2 Hz, 2H), 7.33 (d, J=8.4 Hz, 2H), 4.62 (dd, J=6.9, 6.9 Hz, 1H), 3.77 (s, 3H), 3.70-3.57 (m, 2H), 2.67 (s, 3H), 2.41 (s, 3H), 1.69 ppm (s, 3H); ); LCMS m/z calc. for C₂₀H₂₀ClN₄O₂S [M+H]⁺ 415.1, found: 415.0.

(±)-methyl (R)-2-((S)-4-(4-chlorophenyl)-2,3,9-trimethyl-6H-thieno[3,2-f][1,2,4]triazolo[4,3-a][1,4]diazepin-6-yl)propanoate ((±)-(3S,2R)-ME-JQ1-OMe) (30a). Following general procedure D, compound 30a was obtained using alkylating agent, methyl iodide. Yield 8.9 mg (7%); Following general procedure E, compound 30a can also be obtained from epimerisation of 30b. Isolated Yield: 12 mg (31%); ¹H NMR (500 MHz, CDCl₃): δ=7.34 (d, J=8.7 Hz, 2H), 7.31 (d, J=8.8 Hz, 2H), 4.25 (d, J=10.7 Hz, 1H), 4.07 (qd, J=6.9, 10.7 Hz, 1H), 3.83 (s, 3H), 2.67 (s, 3H), 2.42 (s, 3H), 1.69 (s, 3H), 1.51 ppm (d, J=6.9 Hz, 3H); ¹³C NMR (126 MHz, CDCl₃): δ=176.1, 163.2, 154.5, 149.8, 136.9, 136.7, 132.3, 131.1, 130.9, 130.7, 130.0, 128.8, 60.4, 51.9, 42.6, 15.4, 14.6, 13.2, 12.0; LCMS m/z calc. for C₂₁H₂₂ClN₄O₂S [M+H]⁺ 429.1, found: 429.0.

(±)-methyl (S)-2-((S)-4-(4-chlorophenyl)-2,3,9-trimethyl-6H-thieno[3,2-f][1,2,4]triazolo[4,3-a][1,4]diazepin-6-yl)propanoate ((±)-(3S,2S)-ME-JQ1-OMe) (30b). Following general procedure D, compound 30b was obtained using alkylating agent, methyl iodide. Yield 35.4 mg (29%); ¹H NMR (500 MHz, CDCl₃): δ=7.43 (d, J=8.4 Hz, 2H), 7.33 (d, J=8.5 Hz, 2H), 4.31 (d, J=9.8 Hz, 1H), 3.88 (qd, J=7.2, 9.7 Hz, 1H), 3.72 (s, 3H), 2.64 (s, 3H), 2.41 (s, 3H), 1.70 (s, 3H), 1.62 ppm (d, J=7.2 Hz, 3H); ¹³C NMR (126 MHz, CDCl₃): δ=176.1, 163.9, 155.5, 149.6, 136.95, 136.90, 132.7, 130.8, 130.4, 129.9, 128.8, 58.5, 52.1, 41.2, 15.4, 14.5, 13.2, 11.9; LCMS m/z calc. for C₂₁H₂₂ClN₄O₂S [M+H]⁺ 429.1, found: 429.0.

(±)-methyl (R)-2-((S)-4-(4-chlorophenyl)-2,3,9-trimethyl-6H-thieno[3,2-f][1,2,4]triazolo[4,3-a][1,4]diazepin-6-yl)butanoate ((±)-(3S,2R)-ET-JQ1-OMe) (31a). Following general procedure D, compound 31a was obtained using alkylating agent, ethyl iodide. Yield 20 mg (16%); Following general procedure E, compound 31a can also be obtained from epimerisation of 31b. Isolated Yield: 11 mg (37%); ¹H NMR (500 MHz, CDCl₃): δ=7.35-7.29 (m, 4H), 4.24 (d, J=10.9 Hz, 1H), 3.99 (dt, J=3.7, 10.7 Hz, 1H), 3.84 (s, 3H), 2.66 (s, 3H), 2.41 (s, 3H), 2.23-2.13 (m, 1H), 1.73-1.63 (m, 4H), 1.02 ppm (t, J=7.4 Hz, 3H); ¹³C NMR (126 MHz, CDCl₃): δ=175.5, 163.2, 154.6, 149.8, 136.9, 136.7, 132.3, 131.0, 130.9, 130.6, 129.9, 128.8, 59.5, 51.6, 49.8, 23.4, 14.6, 13.2, 12.0, 11.7; LCMS m/z calc. for C₂₂H₂₄ClN₄O₂S [M+H]⁺ 443.1, found: 443.1.

(±)-methyl (S)-2-((S)-4-(4-chlorophenyl)-2,3,9-trimethyl-6H-thieno[3,2-f][1,2,4]triazolo[4,3-a][1,4]diazepin-6-yl)butanoate ((±)-(3S,2S)-ET-JQ1-OMe) (31b). Following general procedure B, compound 31b was obtained using alkylating agent, ethyl iodide. Yield 29.2 mg (23%); ¹H NMR (500 MHz, CDCl₃): δ=7.42 (d, J=8.4 Hz, 2H), 7.33 (d, J=8.5 Hz, 2H), 4.31 (d, J=10.9 Hz, 1H), 3.83 (dt, J=3.6, 10.1 Hz, 1H), 3.73 (s, 3H), 2.64 (s, 3H), 2.41 (s, 3H), 2.37-2.26 (m, 1H), 1.93-1.82 (m, 1H), 1.69 (s, 3H), 1.05 ppm (t, J=7.5 Hz, 3H); ¹³C NMR (126 MHz, CDCl₃): δ=175.5, 163.9, 155.4, 149.6, 136.95, 136.90, 132.7, 130.8, 130.7, 130.3, 129.9, 128.8, 57.6, 51.9, 47.6, 23.4, 14.5, 13.2, 11.9, 11.2; LCMS m/z calc. for C₂₂H₂₄ClN₄O₂S [M+H]⁺ 443.1, found: 443.1.

(±)-(R)-2-((S)-4-(4-chlorophenyl)-2,3,9-trimethyl-6H-thieno[3,2-f][1,2,4]triazolo[4,3-a][1,4]diazepin-6-yl)propanoic acid ((±)-(3S,2R)-ME-JQ1-OH) (32). Compound 30a (8.2 mg, 19 μmol) was dissolved in THF (400 μL). LiOH (1 mg, 38 μmol) was subsequently dissolved in water (100 μL) and added to the flask. The flask was heated to 35° C. and stirred for 48 h. Water (25 μL) and 0.6 M LiOH solution (25 μL) was added at regular intervals (every 12 h) to assist with the conversion. The conversion of the ester to the acid was monitored by LC-MS. After 100% conversion, the solution was neutralised with 2.0 M HCl solution and freeze dried to afford acid 32. The acid was used as crude for the next step and the yield considered quantitative. Yield: 7.9 mg, (quant.); ¹H NMR (500 MHz, CDCl₃): δ=7.34 (d, J=8.8 Hz, 2H), 7.31 (d, J=8.9 Hz, 2H), 4.25 (d, J=10.6 Hz, 1H), 4.07 (m, 1H), 3.83 (s, 3H), 2.67 (s, 3H), 2.42 (s, 3H), 1.69 (s, 3H), 1.51 ppm (d, J=7.0 Hz, 3H); ¹³C NMR (126 MHz, CDCl₃): δ=175.8, 164.8, 154.6, 150.3, 137.7, 135.7, 132.4, 131.6, 131.4, 130.9, 130.3, 129.0, 59.1, 41.5, 15.6, 14.6, 13.4, 11.8; LCMS m/z calc. for C₂₀H₂₀ClN₄O₂S [M+H]⁺ 415.1, found: 415.1.

(±)-(R)-2-((S)-4-(4-chlorophenyl)-2,3,9-trimethyl-6H-thieno[3,2-f][1,2,4]triazolo[4,3-a][1,4]diazepin-6-yl)butanoic acid ((±)-(3S,2R)-ET-JQ1-OH) (33). Compound 31a (35.2 mg, 80 μmol) was dissolved in THF (1.2 mL). LiOH (4.8 mg, 200 μmol) was subsequently dissolved in water (300 μL) and added to the flask. The flask was heated to 40° C. and stirred for 6 days. Water (50 μL) and 0.65 M LiOH solution (50 μL) was added at regular intervals (every 12 h) to assist with the conversion. The conversion of the ester to the acid was monitored by LC-MS. After 100% conversion, the solution was neutralised with 2.0 M HCl solution and freeze dried to afford acid 33. The acid was used as crude for the next step and the yield considered quantitative. Yield: 34.3 mg, (quant.); ¹H NMR (500 MHz, CDCl₃): δ=7.41 (d, J=8.5 Hz, 2H), 7.32 (d, J=8.7 Hz, 2H), 4.24 (d, J=6.5 Hz, 1H), 3.75-3.70 (m, 1H), 2.69 (s, 3H), 2.43 (s, 3H), 2.03-1.95 (m, 2H), 1.71 (s, 3H), 1.10 ppm (t, J=7.4 Hz, 3H); ¹³C NMR (126 MHz, CDCl₃): δ=175.2, 164.6, 154.9, 150.1, 137.5, 136.1, 132.5, 131.5, 131.2, 130.2, 130.1, 129.0, 58.2, 48.6, 23.8, 14.6, 13.3, 11.9; LCMS m/z calc. for C₂₁H₂₂ClN₄O₂S [M+H]⁺ 429.1, found: 429.1.

(S)-13-((2S,4R)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl (R)-2-((S)-4-(4-chlorophenyl)-2,3,9-trimethyl-6H-thieno[3,2-f][1,2,4]triazolo[4,3-a][1,4]diazepin-6-yl)butanoate (AGB1) (46). Following general procedure F, compound 46 was obtained using alcohol 41 (synthesised according to Klein, V. et al. 2021(supra)) and purified by reverse phase flash column chromatography (15.5 g C18 gold column) using a linear gradient from 5% to 100% MeCN in 0.1% formic acid in water over 12 min to afford AGB1 (46). Yield: 29 mg (30%); ¹H NMR (500 MHz, CDCl₃): δ=8.67 (s, 1H), 7.42 (t, J=5.9 Hz, 1H), 7.36-7.28 (m, 9H), 4.74 (t, J=7.9 Hz, 1H), 4.56-4.49 (m, 3H), 4.44-4.30 (m, 3H), 4.23 (d, J=10.9 Hz, 1H), 4.06 (d, J=11.3 Hz, 1H), 4.01 (d, J=15.7 Hz, 1H), 3.98-3.92 (m, 2H), 3.81-3.72 (m, 2H), 3.69-3.59 (m, 10H), 2.65 (s, 3H), 2.53-2.45 (m, 4H), 2.41 (s, 3H), 2.18-2.09 (m, 2H), 1.73-1.62 (m, 4H), 1.01 (t, J=7.4 Hz, 3H), 0.95 ppm (s, 9H); ¹³C NMR (126 MHz, CDCl₃): δ=174.8, 171.4, 171.1, 170.5, 163.2, 162.9, 154.5, 150.5, 149.9, 148.5, 138.4, 136.9, 136.6, 132.1, 131.8, 131.1, 131.0, 130.9, 130.7, 130.0, 129.6, 128.8, 128.2, 71.3, 70.9, 70.8, 70.6, 70.5, 70.2, 69.3, 63.8, 59.3, 58.7, 57.2, 56.8, 49.7, 43.3, 36.2, 35.3, 26.5, 23.3, 16.1, 14.5, 13.2, 11.9, 11.7; HRMS m/z calc. for C₅₁H₆₄ClN₈O₉S₂ [M+H]⁺ 1031.3921, found: 1031.3961.

2-(3-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)propoxy)ethyl (R)-2-((S)-4-(4-chlorophenyl)-2,3,9-trimethyl-6H-thieno[3,2-f][1,2,4]triazolo[4,3-a][1,4]diazepin-6-yl)butanoate (AGB2) (47). Following general procedure F, compound 47 was obtained using alcohol 44 (synthesised according to Klein, V. et al. 2021(supra)) and purified by HPLC using a linear gradient from 5% to 95% MeCN in 0.1% formic acid over 12 min in water to afford AGB2 (47). Yield: 1.2 mg (10%); ¹H NMR (500 MHz, CDCl₃): δ=8.66 (s, 1H), 7.47 (d, J=7.3 Hz, 1H), 7.39 (d, J=8.4 Hz, 2H), 7.36 (d, J=8.4 Hz, 2H), 7.33 (d, J=8.5 Hz, 2H), 7.29 (d, J=8.8 Hz, 2H), 7.20 (d, J=8.6 Hz, 1H), 5.08 (dq, J=7.2, 7.2 Hz, 1H), 4.78 (t, J=7.9 Hz, 1H), 4.56 (d, J=8.6 Hz, 1H), 4.55-4.51 (m, 1H), 4.49-4.43 (m, 1H), 4.35-4.30 (m, 1H), 4.25 (d, J=10.8 Hz, 1H), 4.12 (d, J=11.3 Hz, 1H), 4.00-3.93 (m, 2H), 3.87 (d, J=15.4 Hz, 1H), 3.80-3.75 (m, 1H), 3.75-3.69 (m, 1H), 3.64-3.58 (m, 5H), 2.65 (s, 3H), 2.58-2.52 (m, 4H), 2.41 (s, 3H), 2.20-2.12 (m, 1H), 2.09 (dd, J=8.3, 13.6 Hz, 1H), 1.93-1.86 (m, 2H), 1.73-1.63 (m, 4H), 1.47 (d, J=6.9 Hz, 3H), 1.06 (s, 9H), 1.02 ppm (t, J=7.4 Hz, 3H); ¹³C NMR (126 MHz, CDCl₃): δ=174.9, 171.8, 170.4, 169.8, 163.2, 154.6, 150.3, 149.9, 148.7, 143.4, 136.9, 136.6, 131.02, 130.99, 130.0, 129.7, 128.8, 126.6, 70.3, 70.2, 69.0, 68.9, 68.0, 63.8, 59.4, 58.5, 57.1, 56.8, 49.8, 49.0, 35.6, 35.2, 30.0, 29.8, 26.7, 23.4, 22.4, 16.2, 14.6, 13.3, 11.9, 11.7; HRMS m/z calc. for C₅₁H₆₄ClN₈O₈S₂ [M+H]⁺ 1015.3972, found: 1015.4197.

(2S,4R)-1-((2S,17R)-2-(tert-butyl)-17-((S)-4-(4-chlorophenyl)-2,3,9-trimethyl-6H-thieno[3,2-f][1,2,4]triazolo[4,3-a][1,4]diazepin-6-yl)-4,16-dioxo-6,9,12-trioxa-3,15-diazanonadecanoyl)-4-hydroxy-N-(4-(4-methylthiazol-5-yl)benzyl)pyrrolidine-2-carboxamide (AGB3) (48). Azide 9 (synthesised according to Zengerle, M, 2015, (supra)) (30 mg, 46 μmol) was dissolved in MeOH (2 mL). A catalytic amount of 10 wt. % Pd/C was added, and the reaction was stirred under an atmosphere of hydrogen for 3 h. The reaction mixture was then filtered through PTFE syringe filters and evaporated to dryness to obtain the desired amine quantitative yields. The resulting amine (7.4 mg, 12 μmol) was dissolved in DMF (96 μL) and added to a solution of ET-JQ1-OH (45, synthesised according to Bond, A. G., 2020, (supra)) (5 mg, 12 μmol), COMU (5.1 mg, 12 μmol) and DIPEA (4.18 μL, 12 μmol) in DMF (96 μL) and stirred at r.t. for 2 h. The mixtures were then concentrated in vacuo and the residues were purified by HPLC using a linear gradient of 5% to 95% MeCN in 0.1% formic acid in water over 12 min to afford AGB3 (48). Yield: 2.2 mg (18%); ¹H NMR (400 MHz, CDCl₃): δ=8.68 (s, 1H), 8.18 (t, J=5.5 Hz, 1H), 7.36 (d, J=8.3 Hz, 2H), 7.31-7.24 (m, 5H), 7.17-7.12 (m, 3H), 4.99 (d, J=4.9 Hz, 1H), 4.85 (t, J=8.2 Hz, 1H), 4.80 (d, J=9.7 Hz, 1H), 4.51 (br. s, 1H), 4.46 (dd, J=7.2, 15.8 Hz, 1H), 4.26 (d, J=10.5 Hz, 1H), 4.19-4.11 (m, 2H), 4.09 (d, J=15.9 Hz, 1H), 3.83-3.63 (m, 15H), 3.50-3.42 (m, 1H), 2.64 (s, 3H), 2.53 (s, 3H), 2.39 (s, 3H), 2.34-2.27 (m, 1H), 2.16 (dd, J=7.5, 13.5 Hz, 1H), 1.96-1.89 (m, 1H), 1.64-1.56 (m, 4H), 1.03-0.96 ppm (m, 12H); ¹³C NMR (101 MHz, CDCl₃): δ=173.8, 171.7, 171.6, 170.3, 163.9, 155.0, 150.3, 149.9, 148.5, 138.6, 136.9, 136.7, 132.0, 131.9, 131.31, 131.28, 130.9, 130.6, 130.1, 129.4, 129.0, 127.5, 71.3, 71.1, 70.75, 70.69, 70.4, 70.3, 59.8, 59.4, 57.7, 56.7, 50.3, 42.9, 39.8, 36.9, 36.0, 26.6, 23.1, 16.2, 14.6, 13.3, 12.0, 11.9; HRMS m/z calc. for C₅₁H₆₅ClN₉O₈S₂ [M+H]⁺ 1030.4081, found: 1030.4589.

(2S,4S)-1-((S)-17-(tert-butyl)-2,2-dimethyl-15-oxo-3,3-diphenyl-4,7,10,13-tetraoxa-16-aza-3-silaoctadecan-18-oyl)-4-hydroxy-N-(4-(4-methylthiazol-5-yl)benzyl)pyrrolidine-2-carboxamide (50). Acid 37 (synthesised according to Klein, V. et al. 2021(supra)) (161 mg, 0.36 mmol), COMU (154 mg, 0.36 mmol), DIPEA (334 μL, 1.92 mmol) were dissolved in DMF (1.92 mL) and stirred at r.t. for 10 min. Amine 49 (synthesised according to Zengerle, M, 2015, (supra)) (112 mg, 0.24 mmol) was added and the reaction was left to stir at r.t. for 2 h. The mixture was then purified by reverse phase flash column chromatography (2×15.5 g C18 column) using a linear gradient from 5% to 100% MeCN in 0.1% formic acid in water over 10 min with a 3 min plateau to afford 50. Yield: 103 mg (50%); ¹H NMR (500 MHz, CDCl₃): δ=8.65 (s, 1H), 7.69-7.65 (m, 4H), 7.57 (t, J=6.1 Hz, 1H), 7.43-7.31 (m, 10H), 7.20 (d, J=9.1 Hz, 1H), 5.52 (d, J=9.8 Hz, 1H), 4.71 (d, J=9.0 Hz, 1H), 4.60 (dd, J=7.0, 14.9 Hz, 1H), 4.52 (d, J=9.1 Hz, 1H), 4.49-4.43 (m, 1H), 4.29 (dd, J=5.1, 14.9 Hz, 1H), 4.01 (d, J=15.6 Hz, 1H), 3.98-3.91 (m, 2H), 3.82-3.77 (m, 3H), 3.70-3.59 (m, 8H), 3.57 (t, J=5.4 Hz, 2H), 2.50 (s, 3H), 2.34 (d, J=14.0 Hz, 1H), 2.19-2.10 (m, 1H), 1.04 (s, 9H), 0.93 ppm (s, 9H); ¹³C NMR (126 MHz, CDCl₃): 172.7, 171.9, 169.9, 150.4, 148.7, 137.5, 135.7, 133.8, 131.6, 131.3, 129.7, 128.3, 127.7, 72.6, 71.3, 71.2, 70.9, 70.8, 70.54, 70.51, 63.5, 60.0, 58.7, 56.6, 43.6, 35.2, 35.1, 30.4, 26.9, 26.4, 19.3, 16.1; LCMS m/z calc. for C₄₆H₆₃N₄O₈SSi [M+H]⁺ 859.4, found: 859.3.

(2S,4S)-1-((S)-2-(tert-butyl)-14-hydroxy-4-oxo-6,9,12-trioxa-3-azatetradecanoyl)-4-hydroxy-N-(4-(4-methylthiazol-5-yl)benzyl)pyrrolidine-2-carboxamide (51). To a solution of compound 50 (51 mg, 59 μmol) in THF (11.9 mL) was added a 1.0 M solution of TBAF in THF (178 μL, 178 μmol). This was left to stir for 6 h. The mixture was then concentrated in vacuo and the residue purified by reverse phase flash column chromatography (15.5 g C18 column) using a linear gradient from 5% to 100% MeCN in 0.1% formic acid in water over 10 min to afford alcohol 51. Yield: 36.6 mg (quant.); ¹H NMR (500 MHz, CDCl₃): δ=8.66 (s, 1H), 8.01 (t, J=5.9 Hz, 1H), 7.38-7.32 (m, 4H), 7.29 (d, J=9.4 Hz, 1H), 4.67 (d, J=8.7 Hz, 1H), 4.64-4.58 (m, 2H), 4.44 (t, J=4.3 Hz, 1H), 4.30 (dd, J=5.1, 15.0 Hz, 1H), 4.04 (d, J=15.6 Hz, 1H), 3.97 (d, J=15.3 Hz, 1H), 3.89 (dd, J=4.2, 10.9 Hz, 1H), 3.84 (d, J=10.7 Hz, 1H), 3.71-3.52 (m, 12H), 3.51-3.44 (m, 1H), 2.50 (s, 3H), 2.26 (d, J=14.3 Hz, 1H), 2.23-2.15 (m, 1H), 0.96 ppm (s, 9H); 172.8, 171.8, 169.8, 150.4, 148.6, 137.7, 131.7, 131.2, 129.6, 128.2, 72.7, 71.2, 71.1, 70.9, 70.5, 70.35, 70.29, 61.7, 60.1, 58.8, 56.5, 43.6, 35.7, 35.5, 26.4, 16.1; LCMS m/z calc. for C₃₀H₄₅N₄O₈S [M+H]⁺ 621.3, found: 621.2.

(S)-13-((2S,4S)-4-hydroxy-2-((4-(4-methylthiazol-5-yl)benzyl)carbamoyl)pyrrolidine-1-carbonyl)-14,14-dimethyl-11-oxo-3,6,9-trioxa-12-azapentadecyl (R)-2-((S)-4-(4-chlorophenyl)-2,3,9-trimethyl-6H-thieno[3,2-f][1,2,4]triazolo[4,3-a][1,4]diazepin-6-yl)butanoate (cis-AGB1) (52). Following general procedure F, compound 52 was obtained using alcohol 51 and purified by HPLC using a linear gradient from 5% to 95% MeCN in 0.1% formic acid in water over 12 min to afford cis-AGB1 (52). Yield: 8.4 mg (51%); ¹H NMR (500 MHz, CDCl₃): δ=8.67 (s, 1H), 7.63 (t, J=5.8 Hz, 1H), 7.38-7.27 (m, 8H), 7.18 (d, J=9.3 Hz, 1H), 5.54 (d, J=10.1 Hz, 1H), 4.75 (d, J=9.1 Hz, 1H), 4.61 (dd, J=7.0, 14.9 Hz, 1H), 4.54 (d, J=9.2 Hz, 1H), 4.49-4.33 (m, 3H), 4.30 (dd, J=5.3, 15.0 Hz, 1H), 4.24 (d, J=10.9 Hz, 1H), 4.01 (d, J=15.8 Hz, 1H), 3.99-3.91 (m, 3H), 3.81 (d, J=11.1 Hz, 1H), 3.79-3.75 (m, 2H), 3.70-3.62 (m, 8H), 2.65 (s, 3H), 2.51 (s, 3H), 2.41 (s, 3H), 2.34 (d, J=14.3 Hz, 1H), 2.21-2.13 (m, 2H), 1.72-1.64 (m, 4H), 1.02 (t, J=7.5, 3H), 0.95 ppm (s, 9H); ¹³C NMR (126 MHz, CDCl₃): δ=174.9, 172.9, 171.7, 169.9, 163.2, 154.5, 150.5, 149.9, 148.6, 137.6, 136.8, 136.5, 132.1, 131.6, 131.2, 131.0, 130.9, 130.5, 130.0, 129.7, 128.8, 128.3, 71.3, 71.2, 70.84, 70.81, 70.5, 69.3, 63.8, 60.0, 59.3, 58.7, 56.5, 49.8, 43.6, 35.30, 35.26, 26.4, 23.3, 16.2, 14.6, 13.3, 12.0, 11.7; HRMS m/z calc. for C₅₁H₆₄ClN₈O₉S₂ [M+H]⁺ 1031.3921, found: 1031.3987.

### Biology.

Cell Culture. HEK293 human embryonic kidney adherent cell line (ATCC, Manassas, VA, USA) were cultured in DMEM (Invitrogen, Carlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. 22RV1; a human prostate carcinoma epithelial adherent cell line (ATCC, Manassas, VA, USA) were cultured in RPMI-1640 (Invitrogen, Carlsebad, CA, USA) supplemented with 10% (v/v) fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. MV-4-11 human acute monocytic leukaemia suspension cell line (ATCC, Manassas, VA, USA) was cultured in IMDM (Invitrogen, arlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity.

CRISPR BromoTag-Brd2 knock-in cell line generation. HEK293 cells were maintained in DMEM (Invitrogen, Carlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. 5×10⁵ HEK293 cells were plated into individual wells of a six-well plate in 1 mL DMEM (Invitrogen, Carlsebad, CA, USA) in the 24 hours leading up to the initiation of the experiment. HEK293 cells transfected the next day in the presence of Cells were transfected using Fugene HD lipofectamine (Madison, Wisconsin, United States) simultaneously with three custom vectors including a px335 custom vector containing a U6-snRNA & Cas9D10A expression cassette, pBABED vector harbouring another U6-sgRNA and puromycin expression cassette and finally a pcDNA5 vector containing an eGFP-P2A-BromoTag-Brd2 donor knock-in sequence. To increase the relative population of cells undergoing homologous recombination, this transfection was performed in the presence of 0.1 μM of the DNA Ligase IV inhibitor SCR7. The following day cells were washed before fresh DMEM media was applied containing 0.1 μM SCR7 and 2 μg/ml of puromycin. This was repeated the following day as cells were washed before fresh DMEM media was applied containing 0.1 μM SCR7 and 2 μg/mL of puromycin. The following day the cells were washed for the third time, and fresh media absent of both SCR7 and puromycin was applied to allow for recovery. The following day, HEK293 cells were then subsequently washed the next day washed and fresh DMEM has applied again, containing 2.5 μg/mL puromycin and 0.1 μM SCR7. This process was continued for a further two days. Cells were then washed with PBS before recovery in DMEM was performed for a further 20 days. Cells were subsequently prepared for FACS sorting.

Fluorescence-Activated Cell Sorting of GFP Positive CRISPR Knock-In BromoTag-Brd2 HEK293 cells. HEK293 cells that had undergone CRISPR lipofection and selection in the previous stage were subsequently trypsinised using tTrypsin-EDTA (0.05%), phenol red (Thermo Fisher, Waltham, MA, USA). Once in suspension, the trypsin-cell mixture was neutralised with FBS (Thermo Fisher, Waltham, MA, USA). Cells were pelleted at 1500 rpm for 5 min. The cell pellet produced was subsequently resuspended in DMEM media supplemented with 1% FBS at concertation of 5×10⁶ cells per mL. Wild-type HEK293 cells were used as a baseline control for GFP expression. Single cell clones were generated by Fluorescence Activated Cell Sorting (FACS) using an SH800 cell sorter from Sony Biotechnology of the Dundee University Flow Cytometry and Cell Sorting Facility. A 488 nm laser was used for excitation of fluorescence and generation of light scatter. Forward angle light scatter (FSC) and back scatter (BSC) were detected using 488±17 nm band pass filters. Cells were distinguished from debris on the basis of FSC-Area(A) and SSC-A measurements. Single cells were distinguished from doublets and clumps on the basis of FSC-A and FSC-Width (W) measurements. GFP fluorescence was detected using a 525±50 nm band pass filter and autofluorescence was detected using a 600±60 nm band pass filter. GFP positive cells were identified by first assessing the background GFP and autofluorescence of a control sample of cells which did not express GFP. Using the measurements for GFP and autofluorescence of this sample, a collection gate was set which identified GFP positive cells. The samples to be sorted were then analysed and GFP positive cells sorted for collection.

A single GFP +ve cell was sorted into each well of a 3×96 well plates 3×96 well plates (Thermo Fisher, Waltham, MA, USA) in 200 μL of 50% filtered pre-conditioned media from healthy cells & 50% fresh DMEM containing 10% FBS and 1% (v/v) penicillin/streptomycin (pen/strep) (#15140122, Thermo Fisher, Waltham, MA, USA) and stored at 37° C., 5% CO₂, and 95% humidity for two weeks. After two weeks, all visible colonies were expanded and subsequently frozen down.

Genomic DNA extraction. Brd2 expression in the post expanded cell lines was analysed via western blot and suspected cell lines were subsequently harvested for genomic extraction. Cells were plated at a density of 2×10⁶ cells in a well of a 10 cm plate. Forty-eight hours later, the cells were Trypsinised using Trypsin-EDTA (0.05%), phenol red (Thermo Fisher, Waltham, MA, USA). Once in suspension, the trypsin-cell mixture was neutralised with FBS (Thermo Fisher, Waltham, MA, USA). Cells were pelleted at 1500 rpm for 5 min. The remaining pellet of each clone underwent genomic extraction following a solution-based extraction approach using PROMEGA's Wizard® Genomic DNA Purification Kit following the instruction provided. The DNA extracted was subsequently analysed using a Nanodrop spectrophotometer and stored at −20° C. prior to use.

Junction PCR. Junction PCR was performed using the following primers: Forward, AGTCTGTCCACCCCCTCTAC, and Reverse, ACTCCACTCCACCGTCAAAC. Extracted Genomic DNA from the previous step was used as the template for a subsequent PCR reaction. Using Phusion high fidelity polymerase and 250 ng of template DNA of either clone or HEK293 Wild-type genomic DNA a 30-cycle PCR was run with a melting temperature of 98° C., an annealing temperature of 60° C., and a two-minute elongation step at 72° C. The product of these PCR's were then subsequently run on a 2% agarose gel containing 1× Sybersafe DNA staining reagent (Invitrogen, Carlsebad, CA, USA) in 1×DNA loading dye (Thermo Fisher, Waltham, MA, USA) along with 1× Generuler 1 Kb plus DNA marker (Thermo Fisher, Waltham, MA, USA) at 100 volts for 30 min. The run gel was imaged using a Bio-Rad Gel Doc system (Bio-Rad, Hercules, California).

Genotyping. Using the agarose gel containing the junction PCR product, appropriately sized bands from that agarose gel were harvested using a UV imager and a scalpel. The bands chosen corresponded to the HEK293 wild-type Brd2 junction product 1 kb, the BromoTag-Brd2 clone wild-type Brd2 junction product 1 kb, and the BromoTag-Brd2 clone Knock-in junction product 2 kb. The excised bands were subsequently removed from the agarose using a Monarch® DNA Gel Extraction Kit (NEB, Ipswich, Massachusetts). Following extraction, the PCR product was ligated into blunt-end vectors using Strataclone Blunt PCR Cloning kit (Agilent, Santa Clara, California) and subsequently transformed into Cre recombinase expressing E. coli (Agilent, Santa Clara, California) and plated onto kanamycin 50 μg/mL agar plates. A day following plating, visible colonies were picked and grown for 16-hours in 5 ml's of kanamycin 50 μg/mL containing LB standard formula. The subsequent overnight bacterial growth underwent plasmid miniprep extraction using Monarch® Plasmid Miniprep Kit (NEB, Ipswich, Massachusetts). The vector product recovered after extraction were subsequently analysed using a nanodrop spectrophotometer. These products underwent sequencing using an Applied Biosystems 3730 DNA analyser using commercially available M13-Forward, M13-Reverse, and eGFP-C1-Forward primers. The sequencing was performed by Dundee universities DNA sequencing and services. The raw data from sequencing was subsequently analysed using Jalview software.

Dose-response Degradation Assays. All dose-response degradation assays were performed on the genotype verified heterozygous BromoTag-Brd2 HEK293 cell line. Heterozygous BromoTag-Brd2 HEK293 cells were plated at a density of 5×10⁵ cells per well of a six healthy plate a day before initiation of the titration experiment. PROTAC compounds were dissolved in DMSO at a concentration of 10 mM, from these stock concentrations, PROTAC compounds were diluted to appropriate concentrations using DMSO in the range of 10 μM-1 nM. The compounds were then added to a 2 mL of DMEM (Invitrogen, Carlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) and added to the cells at initiation of the experiment. Control compounds such as MZ1, cis-MZ1 were similarly dissolved in DMSO to the appropriate concentration. All titration experiment were performed for a total of 6 hours prior to being harvested and were kept at 37° C., 5% CO₂, and 95% humidity once treatment was applied until right before harvesting. Cells were washed twice with PBS before being harvested.

Time-course Degradation Assay. Time-course degradation assays using PROTACs AGB1, AGB2, and AGB3 were performed on the genotype verified heterozygous BromoTag-Brd2 HEK293 cell line. Heterozygous BromoTag-Brd2 HEK293 cells were plated at a density of 5×10⁵ cells per well of a 6 healthy plate a day prior to initiation of the Timecourse assay. PROTAC's AGB1 & AGB2 were diluted in DMSO to a concentration of 1 mM prior to being further diluted 1:2000 in 2 mL of DMEM to a concentration of 500 nM per timepoint. PROTAC AGB3 was diluted in DMSO to a concentration of 2 mM prior to being further diluted 1:2000 in 2 mL of DMEM to a concentration of 1 μM per timepoint. Timepoint range was between 0-36 h. Treatment was applied in a staggered fashion to enable all timepoints to be harvested at the same time.

Recovery Assay. A recovery assay was performed using 200 nM AGB1 over a 72 h period. This was performed in the genotype verified heterozygous BromoTag-Brd2 HEK293 cell line. Heterozygous BromoTag-Brd2 HEK293 cells were plated at a density of 5×10⁵ cells per well of a six-well plate a day before initiating the recovery assay. On the experiment day, cells were washed with PBS before fresh DMEM was applied to contain either DMSO or AGB1 200 nM. During treatment, cells were kept at 37° C., 5% CO₂, and 95% humidity. After three hours, the recovery and vehicle control condition cells were rewashed with PBS before fresh DMEM was applied absent 200 nM AGB1 or DMSO. As for the positive control condition, they were left with 200 nM AGB1 for the remainder of the treatment time.

Acquisition of the polyclonal Brd4^{BD2 L387A} antibody. To generate a polyclonal Brd4^{BD2 L387A} antibody, a sheep was immunised with 0.35 mg of His-Brd4^{BD2 L387A} domain protein purified as previously described (Gadd, M. S. et al., Nat. Chem. Biol. 2017, 13 (5), 514-521; Baud, M. G. J. et al., Science 2014, 346 (6209), 638-641) and prepared in a buffer containing 20 mM HEPES pH7.5, 0.5 M NaCl, 1 mM DTT. This was followed by four further injections 28 days apart. Bleeds were performed seven days after each injection. Antibodies were affinity-purified from serum and eluted with 50 mM Glycine pH 2.5, neutralised with 1 M Tris pH 8 and dialysed into PBS buffer using His-Brd4^{BD2} L^{387A} protein.

Competition Assay. Heterozygous BromoTag-Brd2 HEK293 cells were plated in six-well plates at a density of 5×10⁵ cells per well in 2 mL DMEM media. At initiation of experiment, cells were treated with either 3 μM MLN4924, 50 μM MG132, 10 μM VH298, 10 μM ET-JQ1-OMe, or 0.1% DMSO. After 1 h, 200 nM of AGB1 was added to the previously compound treated cells. After a following 3 h the cells were harvested for subsequent processing via western blot. Each treatment was performed in tandem to produce two technical repeats per condition. The 6 well plates were incubated for 4 h at 37° C. and 5% CO₂ throughout the experiment.

Western Blotting. All cells were harvested on ice with RIPA lysis, and extraction buffer (ThermoFisher Scientific, 89901) supplemented with protease inhibitor cocktail (Merck, 11697498001) and Benzonase® Nuclease (Sigma, E1014) before being store at −20° C. prior to use. Total protein quantity was determined using the BCA protein assay (#23225, Pierce, Rockford, Illinois). Protein concentration was determined using the BCA assay (ThermoFisher Scientific, 23225). Samples were then prepared and loaded onto NuPAGE™ 4-12% Bis-Tris Midi gels (ThermoFisher Scientific, WG1403A), followed by the transfer of the proteins onto nitrocellulose membranes (EMD Millipore). The membranes were blocked for 1 h prior to incubation with the primary antibodies using 5% Milk TBST. Membranes were probed for Brd2 (Abcam, Ab139690, 1:1000), Brd3 (Abcam, Ab50818, 1:4000), Brd4 (Abcam, Ab128874, 1:1000) or our polyclonal Brd4^{BD2 L387A} antibody. Following overnight incubation with the primary antibodies at 4° C., the membranes were incubated with secondary antibodies (Anti-rabbit, Abcam AB216773, 1:5000 or anti-mouse, Abcam AB216774, 1:5000) and hFAB™ Rhodamine Anti-Tubulin Antibody (Biorad, 12004165, 1:10000) for one h and then imaged with a Bio-Rad imager (LI-COR Biosciences). All western blots were analysed for band intensities using Image Lab from Bio-Rad (LI-COR, Biosciences). The data extracted from these blots were then plotted and analysed using Prism (v. 8.2.0, GraphPad).

Cell Viability Assay. MV-4-11 cells were plated at a density of 2×10⁴ cell per well of a 96 well white-bottom plate, and left to grow overnight in 50 μl of IMDM (Invitrogen, arlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. The cells were then treated with 50 μl of IMDM supplemented with 2× compound treatment, including DMSO, AGB1, cis-AGB1, MZ1, cis-MZ1 or Staurosporine. These cells were then left to incubate at 37° C., 5% CO₂, and 95% humidity for one day prior to undergoing spectrophotometric analysis. 22RV1 cells were plated at a density of 2×10⁴ cell per well of a 96 well white-bottom plate, and left to grow overnight in 100 μL of RPMI-1640 (Invitrogen, Carlsebad, CA, USA) supplemented with 10% (v/v) fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. The cells were then washed twice in PBS before 100 μl of fresh RPMI-1640 media supplemented with 1× compound treatment; including DMSO, AGB1, cis-AGB1, MZ1, cis-MZ1 or Staurosporine. These cells were then left to incubate at 37° C., 5% CO₂, and 95% humidity for two days prior to undergoing spectrophotometric analysis. HEK293 wild-type cells were plated at a density of 2×10⁴ cell per well of a 96 well white-bottom plate, and left to grow overnight in 100 μL of DMEM (Invitrogen, Carlsebad, CA, USA) supplemented with 10% (v/v) Fetal bovine serum (FBS) (Thermo Fisher, Waltham, MA, USA) and 1% (v/v) penicillin/streptomycin (pen/strep) (#15140122, Thermo Fisher, Waltham, MA, USA) at 37° C., 5% CO₂, and 95% humidity. The cells were then washed twice in PBS before 100 μl of fresh DMEM supplemented with 1× compound treatment, including DMSO, AGB1, cis-AGB1, MZ1, cis-MZ1 or Staurosporine. These cells were then left to incubate at 37° C., 5% CO₂, and 95% humidity for two days prior to undergoing spectrophotometric analysis. All cell lines were were treated with compounds in duplicate (triplicate for DMSO controls) at a 1× concentration in 0.1% DMSO. Compounds were serially diluted to produce a 7 point, 10-fold titration. Cells were treated with 50:100 μL of compound for a final concentration of 10 μM:10 μM in 0.1% DMSO. At the point of spectrometric analysis cells were treated with 100 μL of Promega CellTiter-Glo® 2.0 Cell Viability Assay reagent. Plates were subjected to 2 mins on an orbital shaker to encourage lysis and left for a further 5 mins to reach peak luminescence. Luminescence was then recorded on a BMG Labtech PHERAstar luminescence plate reader with recommended settings. Data extracted from this analysis was analysed with Graphpad Prism (v. 8.2.0, GraphPad) and normalized to the DMSO vehicle control. EC₅₀ values were derived from these plots.

Sample processing, TMT labelling and fractionation. CRISPR modified BromoTag-Brd2 HEK293 cells were seeded 5×10⁶ cells on a 100 cm plate 24 h before treatment. Cells were treated with either DMSO, 1 μM AGB1 or 1 μM cis-AGB1. 2 h post-treatment, the cells were washed twice with PBS. Cells were lysed in 150 μL of 100 mM TEAB, 5% (w/v) SDS. The lysate was sonicated for 10 s and then centrifuged at 15,000 g for 5 min with the supernatant collected post-centrifugation. Samples were then quantified using a micro-BCA protein assay kit (Thermo Fisher Scientific); 300 μg of each sample were then reduced, alkylated and then digested using Strap mini protocol (Protifi) protocol as described by the manufacturer (protifi) with some modification. Samples were double digested with trypsin (1:40) first overnight then for another 6 hrs with the same ratio (1:40) in 50 mM TEAB buffer. Peptides were quantified using a quantitative fluorometric peptide assay (Thermo Fisher Scientific). The samples (90 μg each) were labelled with a TMT 10-plex Isobaric Label Reagent set (Thermo Fisher Scientific) as per the manufacturer's instructions. After labelling, samples were checked for labelling efficiency, then mixed, desalted, and dried in a speed-vac at 30° C. Samples were re-dissolved in 200 μl ammonium formate (10 mM, pH 9.5) and peptides were fractionated using High pH RP Chromatography. A C18 Column from Waters (XBridge peptide BEH, 130 Å, 3.5 μm 2.1×150 mm, Waters, Ireland) with a guard column (XBridge, C18, 3.5 μm, 2.1×10 mm, Waters) were used on an Ultimate 3000 HPLC (Thermo-Scientific). Buffers A and B used for fractionation consist, respectively, of (A) 10 mM ammonium formate in milliQ water pH 9.5 and (B) 10 mM ammonium formate, pH 9.5 in 90% acetonitrile. Fractions were collected using a WPS-3000FC auto-sampler (Thermo-Scientific) at 1 min intervals. Column and guard column were equilibrated with 2% Buffer B for twenty minutes at a constant flow rate of 0.2 ml/min. 100 μl of TMT labelled peptides were injected onto the column, and the separation gradient was started 1 min after sample was loaded onto the column. Peptides were eluted from the column with a gradient of 2% Buffer B to 20% Buffer B in 6 minutes, then from 20% Buffer B to 45% Buffer B in 51 minutes, finally from 45% buffer B to 100% Buffer B within 1 min. The Column was washed for 15 minutes in 100% Buffer B. The fraction collection started 1 minute after injection and stopped after 80 minutes (total 80 fractions, 200 μl each). To acidify the eluting peptides, 30 μl of 10% formic acid was added to each of the 80 fractionation vials. Total number of fractions concatenated was set to 20.

LC-MS Analysis. Analysis of peptides was performed on a Q-exactive-HF (Thermo Scientific) mass spectrometer coupled with a Dionex Ultimate 3000 RS (Thermo Scientific). LC buffers were the following: buffer A (0.1% formic acid in Milli-Q water (v/v)) and buffer B (80% acetonitrile and 0.1% formic acid in Milli-Q water (v/v). Aliquots of 7 μL of each sample were loaded at 10 μL/min onto a trap column (100 μm×2 cm, PepMap nanoViper C18 column, 5 μm, 100 Å, Thermo Scientific) equilibrated in 0.1% TFA. The trap column was washed for 3 min at the same flow rate with 0.1% TFA and then switched in-line with a Thermo Scientific, resolving C18 column (75 μm×50 cm, PepMap RSLC C18 column, 2 μm, 100 Å). The peptides were eluted from the column at a constant flow rate of 300 nl/min with a linear gradient from 5% buffer B (for Fractions 1-10, 7% for Fractions 11-20) to 35% buffer B in 125 min, and then FROM 35% buffer B to 98% buffer B in 2 min. The column was then washed with 98% buffer B for 20 min and re-equilibrated in 5% or 7% buffer B for 17 min. The column was kept all the time at a constant temperature of 50° C. Q-exactive HF was operated in data dependent positive ionisation mode. The source voltage was set to 2.25 Kv and the capillary temperature was 250° C. A scan cycle comprised MS1 scan (m/z range from 335-1600, with a maximum ion injection time of 50 ms, a resolution of 120 000 and automatic gain control (AGC) value of 3×10⁶) followed by 15 sequential dependent MS2 scans (resolution 60000) of the most intense ions fulfilling predefined selection criteria (AGC 1×10⁵, maximum ion injection time 200 ms, isolation window of 0.7 m/z, fixed first mass of 100 m/z, spectrum data type: centroid, intensity threshold 5×10⁴, exclusion of unassigned, singly and >6 charged precursors, peptide match preferred, exclude isotopes on, dynamic exclusion time 45 s). The HCD collision energy was set to 32% of the normalized collision energy. Mass accuracy is checked before the start of samples analysis.

Peptide and Protein Identification. The raw data files for all fractions were merged and searched against the Uniprot-human-canconical database by MaxQuant software v.1.6.0.16 for protein identification and TMT reporter ion quantitation. The following MaxQuant parameters were used: enzyme used, trypsin/P; maximum number of missed cleavages equal to two; precursor mass tolerance equal to 10 ppm; fragment mass tolerance equal to 20 ppm; variable modifications, oxidation (M), acetyl (N-term), deamidation (NQ), Gln→pyro-Glu (Q N-term); fixed modifications, carbamidomethyl (C). The data were filtered by applying a 1% false discovery rate followed by exclusion of proteins with fewer than two unique peptides. Quantified proteins were filtered if the absolute fold change difference between the three DMSO replicates was ≥1.5.

Protein Expression and Purification. VCB was expressed and purified as described previously (Gadd, M. S. et al., Nat Chem Biol 2017, 13 (5), 514-521). Briefly, N-terminally Hiss-tagged VHL (54-213), ElonginC (17-112) and ElonginB (1-104) were co-expressed in E. coli and the complex was isolated using Ni-affinity chromatography using TEV protease to remove His6 Tag. The complex was further purified by anion exchange followed by gel filtration chromatography. Brd4-BD2^{L387A}was expressed and purified as described previously (Gadd, M. S., 2017 (supra); Baud, M. G. J., 2014 (supra)). Briefly, N-terminally His₆-tagged Brd4-BD2^{L387A} (333-460) was expressed in E. coli and isolated by Ni-affinity chromatography using TEV protease to remove His6 Tag followed by gel filtration chromatography.

Fluorescence Polarization Binding Assay. Fluorescence Polarization (FP) competitive binding assays were performed as described previously (Van Molle et al., Chem. Biol. 2012, 19 (10), 1300-1312; Roy, M. J. et al. ACS Chem Biol. 2019, 14 (3), 361-368) with all measurements taken using a PHERAstar FS (BMG LABTECH) with fluorescence excitation and emission wavelengths (A) of 485 and 520 nm, respectively. Assays were run in triplicate using 384-well plates (Corning 3820), with each well solution containing 15 nM VCB protein, 10 nM 5,6-carboxyfluorescein (FAM)-labelled HIF-1a peptide (FAM-DEALAHypYIPMDDDFQLRSF, “JC9”) and decreasing concentrations PROTACs (14-point, 2-fold serial dilution starting from 20 μM PROTAC) or PROTACs:bromodomain (14-point, 2-fold serial dilution starting from 20 μM PROTAC: 50 μM bromodomain into buffer containing 10 μM of bromodomain). All components were dissolved from stock solutions using 100 mM Bis-Tris propane, 100 mM NaCl, 1 mM DTT, pH 7.0, to yield a final assay volume of 15 μL. DMSO was added as appropriate to ensure a final concentration of 2% v/v. Control wells containing VCB and JC9 with no compound (zero displacement), or JC9 in the absence of protein (maximum displacement) were also included to allow for normalization. Percentage displacement values were obtained by normalization of controls were plotted against Log[Compound]. IC₅₀ values were determined for each titration using nonlinear regression analysis with Prism (v. 9.1.0, GraphPad). Kᵢ values were back-calculated from the K_{d} for JC9 (˜1.5-2.5 nM, determined from direct binding) and fitted IC₅₀ values, as described previously (Van Molle, 2012 (supra); Soares, P. et al., J Med Chem. 2018, 61 (2), 599-618). Cooperativity values (a) for each PROTAC were calculated using the ratio: α=binary K_{d} (−bromodomain)/ternary K_{d} (+bromodomain).

Plasma Stability. Plasma stability studies were outsourced and undertaken by Shanghai ChemPartner Co., Ltd. Buffer preparation: A solution of 0.05 M sodium phosphate and 0.07 M NaCl buffer at pH 7.4 was made by dissolving 14.505 g/L Na₂HPO₄·12H₂O, 1.483 g/L NaH₂PO₄·2H₂O and 4.095 g/L NaCl in deionized water and the pH adjusted with phosphoric acid. Plasma preparation: Frozen mouse plasma was thawed by placing at 37° C. quickly. The thawed plasma was centrifuged at 3,000 rpm for 8 min to remove clots and the supernatant was pooled to be used as the plasma in the experiment. The plasma (pH 7.4-8.0) was stored on ice until used. AGB1 (46) and reference compound procaine were prepared as spiking solution (0.02 mM compound in 0.05 mM sodium phosphate buffer with 0.5% BSA (bovine serum albumin), 4% v/v/DMSO). Plasma and spiking solutions were pre-warmed at 37° C. for 5 min, then 10 μL of pre-warmed spiking solution B were added into the wells designated for all the time points (5, 15, 30, 45, 60 min). For 0-min, 400 μL of acetonitrile containing internal standards (Imipramine, Glipizide) were added to the wells of 0-min plate and then 90 μL of plasma was added. For the time points (0, 5, 15, 30, 45, 60 min), 90 μL of pre-warmed plasma was added to start timing. At 5, 15, 30, 45, 60 min, 400 μL of acetonitrile containing internal standard (Imipramine, Glipizide) were added to the wells of corresponding plates, respectively, to stop the reaction. After quenching, the plates were shaken at the vibrator (IKA, MTS 2/4) for 10 min (600 rpm/min) and then centrifuged at 5594 g for 15 min (Thermo Multifuge×3R). 50 μL of the supernatant from each well of the centrifuged plate were transferred into a new 96-well sample plate containing 50 μL of ultra-pure water (Millipore, ZMQS50F01) for LC/MS analysis (LC-MS/MS-49(AP16500+), UPLC-MSMS-32(Triple Quad 6500+)). Data was analysed with Microsoft Excel.

In Vivo PK Profiling. Pharmacokinetic profiling was outsourced and undertaken by Shanghai ChemPartner Co., Ltd. Six to eight-week-old, C57BL/6 male mice purchased from Jihui Laboratory Animal Co. LTD were used in the study. AGB1 (46) was formulated in 5% DMSO+5% Solutol HS 15+90% Saline at 1 mg/mL. For IV injections, 5 mg/kg of AGB1 (46) was administered into the tail vein of 9 mice. For SC injections, 5 mg/kg of AGB1 (46) was administered via subcutaneous injection in 9 mice. The animals were restrained manually at the designated time points (0.083, 0.25, 0.5, 1, 2, 4 and 8 h), approximately 110 μL of blood sample was collected via facial vein into K₂EDTA tubes. Three mice per time point were used resulting in a total of 18 mice. Blood sample was put on ice and centrifuged at 2000 g for 5 mins to obtain plasma sample within 15 min. Plasma samples were stored at approximately −70° C. until analysis. A 30 μL aliquot of plasma was added with 200 μL internal standard (Diclofenac, 40 ng/mL) in 1% formic acid in MeCN. The mixture was then vortexed for 1 min and then centrifuged for 10 min at 5800 rpm. 100 μL of supernatant was transferred to a new plate. 0.5 μL of solvent was injected to LC-MS/MS. LC-MS/MS instrument used: SCIEX LC-MS/MS-45 (Triple Quad 6500+). Data was analysed by WinNonLin and Microsoft Excel.

### Results and Discussion

Background and Design Rationale of the BromoTag. To design the BromoTag, we hypothesized that we could leverage our potent and selective BET bromodomain recruiting PROTAC MZ1 (1, FIG. 1A) and its target BET bromodomain as degron tag (Zengerle, M, 2015, (supra)). Our extensive mechanistic and structural characterization of MZ1 mode of action highlighted that MZ1, with its E3 ligase VHL bound, formed the most stable, cooperative and long-lived ternary complex with the second bromodomain of Brd4 (Brd4^{BD2}), despite consisting of the pan-selective BET bromodomain ligand (+)-JQ1 (3, FIG. 1B). This preferential recruitment leads to productive ubiquitination and preferential degradation of endogenous Brd4 (Gadd, M. S., 2017 (supra); Roy, M. J., 2019 (supra)). These findings suggested that Brd4^{BD2} could provide an attractive degron tag for ligand-induced degron technologies, however the use of MZ1 would induce confounding downstream effects from its potent induced degradation of all endogenous BET proteins.

To circumvent this limitation, we leveraged engineered variants of BET bromodomains that we previously described, which create a cavity (or ‘hole’) in the BET bromodomains enabling allele-selective binding by a bulkier synthetic BET ligand bearing a ‘bump’ (Baud, M. G. J., 2014 (supra)). Our previous, extensive work developing such a “bump-and-hole” approach identified a Leu residue in the ligand binding site, strictly conserved across all BET family members. Using site directed mutagenesis, this Leu was mutated to a smaller Ala or Val to create a hole that maintained domain stability and ligand-binding capacity. Simultaneously, the pan-selective BET inhibitor I-BET762 (4, FIG. 1B) was modified by introducing a methyl or ethyl “bump” to yield ME and ET (5 and 6 respectively, FIG. 1B), and later on 9-ME-1 and 9-ET-1 (7 and 8 respectively, FIG. 1B), which differ by having a methoxy shift from the 8′ to 9′ position of the fused phenyl ring in the I-BET762 scaffold (Runcie, A. C., 2018 (supra); Baud, M. G. J., 2014 (supra)). The steric “bump” was accommodated into the newly formed hole while clashing with the wild-type protein, allowing us to engineer exquisite allele-selectivity within BET bromodomains (Runcie, A. C., 2018 (supra); Baud, M. G. J., 2014 (supra)). We therefore reasoned that using such bumped BET ligands within the context of the MZ1 PROTAC degrader would enable selective degradation of target proteins fused to a mutant Brd4^{BD2} domain, and without detrimental degradation of endogenous wild-type BET proteins. Such bespoke ‘Bump-&-Hole’-PROTACs (B&H-PROTACs) would therefore offer a complementary, generalizable system of PROTAC-inducible degron tag technology.

Development of a Knock-in Cell Line with BromoTag fused to endogenous Brd2 using CRISPR. To establish the BromoTag platform and support degrader structure-activity relationships (SAR) to identify the best compound, we sought a practical and simple system that would enable us to best triage not only the degradation efficiency, but also the selectivity profile of our degraders. To this end, the endogenous BET family protein Brd2 was chosen as a model target, due to the availability of well-established antibody for Brd2 detection, and expression of a single protein isoform as well detected band in the western blot (Filippakopoulos, P.; Knapp, S., Nat Rev Drug Discov. 2014, 13 (5), 337-356). Because Brd2 contains endogenous bromodomains, and is degraded by 1 and other BET PROTACs, we reasoned that a heterozygous knock-in cell line would allow us to monitor simultaneously both on-target degradation (BromoTagged-Brd2), alongside off-target degradation (untagged Brd2) using the same antibody. Together with potential off-target degradation of the other BET proteins Brd3 and Brd4, this system would thus enable us to best monitor protein degradation selectivity. We therefore decided to add the BromoTag at the N-terminus of the endogenous Brd2 gene locus using CRISPR knock-in methodologies, thereby yielding a chimeric protein bearing three bromodomains (the exogenous BromoTag, in addition to the endogenous Brd2^{BD1} and Brd2^{BD2}). Here forth, we refer to on-target activity as the degradation of BromoTag-Brd2, and off-target activity as any degradation of both untagged Brd2 and endogenous Brd3 and Brd4.

The BromoTag itself was designed based on our previous work to develop a bump and hole (B&H) strategy for BET family proteins (Baud, M. G. J., 2014 (supra)). To maximize our chances of producing a successful and complementary degron for our MZ1-based B&H-PROTACs, we chose to use that Brd4^{BD2 L387A} as the degron “BromoTag” construct (comprising residues 368-440 of human Brd4, size −15 kDa). The specific bromodomain Brd4^{BD2} was chosen because it forms the strongest and most cooperative ternary complex with 1 and VCB (VHL:ElonginC:ElonginB), facilitating productive ubiquitination and rapid, potent degradation of endogenous Brd4 by MZ1 (Gadd, M. S., 2017 (supra)). Moreover, the specific L387A mutation on Brd4^{BD2}was chosen instead of L387V because it shows greatly reduced binding affinity for acetylated histone tail partners compared to the wild-type or L-V domain (Runcie, A. C., 2018 (supra)), suggesting it would be less likely to introduce unwanted neo functionality or protein-protein interactions when used as a tag.

At the outset of the project, we chose HEK293 cells for our CRISPR knock-in experiments to establish a model BromoTag cell line due to their ease of transfection, good level of CRISPR efficiency (Tovell, H. et al., ACS Chem Biol. 2019, 14 (5), 882-892), and high level of expression of all three BET proteins. HEK293 cells were transfected simultaneously with three plasmid constructs, two harbouring cas9_{D10A} and both N-terminal Brd2 specific gRNAs. The other plasmid held the knock-in sequence of the Brd4^{BD2 L387A} BromoTag. The full knock-in construct contained in the 5′-3′ direction an eGFP fluorescent marker, a P2A splice sequence followed subsequently with the Brd4^{BD2} L387A sequence (FIG. 2A). Post transfection, cells underwent fluorescence-activated cell sorting (FACS) to identify GFP expressing single cells denoting successful integration of the knock-in construct (FIG. 2B). Cells were expanded from GFP expressing single cells, and an optimal heterozygous knock-in clone was identified and chosen. A subsequent junction PCR was undertaken demonstrating successful heterozygous integration of the BromoTag N-terminally to Brd2 (FIG. 2C). Since HEK293 is a hypo-triploid cell line, we suspect that the disparity in band intensity present in the junction PCR for the wild-type over the knock-in is due to single allele integration of our knock-in, leaving potentially two wild-type non-modified alleles (FIG. 2C). This heterozygous clone was further validated via western blot using a Brd2 antibody and by independently observing BromoTag-Brd2 expression using an antibody against the BromoTag (FIG. 2D). This antibody was raised in-house using a Brd4BD2^{L387A} protein recombinantly expressed in E. coli as the antigen. This heterozygous BromoTag-Brd2 HEK293 cell line was then subsequently genotyped showing successful in-frame knock-in of the eGFP-P2A-BromoTag knock-in at the N-terminus of Brd2. This cell line will now be referred to as BromoTag-Brd2 HEK293 herein.

Development of first generation, I-BET762-based B&H-PROTACs. In order to combine both bump-&-hole and PROTAC technologies, we set out to make an initial series of B&H-PROTACs, using MZ1 as a template and replacing its BET targeting ligand with a variety of bumped-I-BET762 derivatives we had previously developed (Runcie, A. C., 2018 (supra); Baud, M. G. J., 2014 (supra)). We first inspected our ternary complex crystal structure between Brd4^{BD2}, 1 and VCB (FIG. 3A) and superposed onto Brd4^{BD2} the co-crystal structures of bumped-I-BET chemical probes, 6 and 7 in complex with Brd2^{BD2 L383A} (FIG. 3B) and Brd2^{BD2 L383V} (FIG. 3C), respectively. The chemical structures of 1 and 6 (FIG. 3B), and 1 and 7 (FIG. 3C) adopt a very similar binding mode, with the carbon adjacent to the methyl ester bearing ethyl or methyl bump in 6 and 7 respectively, aligning nicely with the non-bumped bromodomain-binding portion of 1. With these structural insights, we proceeded to synthesise the first generation I-BET-based B&H-PROTACs (Scheme 1).

We first reduced VH032-PEG3 azide 9 (Zengerle, M, 2015, (supra)) with a suspension of 10% palladium on carbon in methanol under an atmosphere of hydrogen gas to yield terminal amines which were then coupled to racemic I-BET762 derived acids, 10-13, via standard amide coupling conditions with 1-[bis(dimethylamino)methylene]-1H-1,2,3-triazolo[4,5-b]pyridinium 3-oxid hexafluorophosphate (HATU), 1-hydroxy-7-azabenzotriazole (HOAt) and diisopropylethylamine (DIPEA) in dimethylformamide (DMF) or dichloromethane (DCM) to yield bumped-I-BET PROTACs, DAT487-489 (15-17) and the non-bumped control, MZP-15 (14), each as mixtures of two diastereomers (Scheme 1).

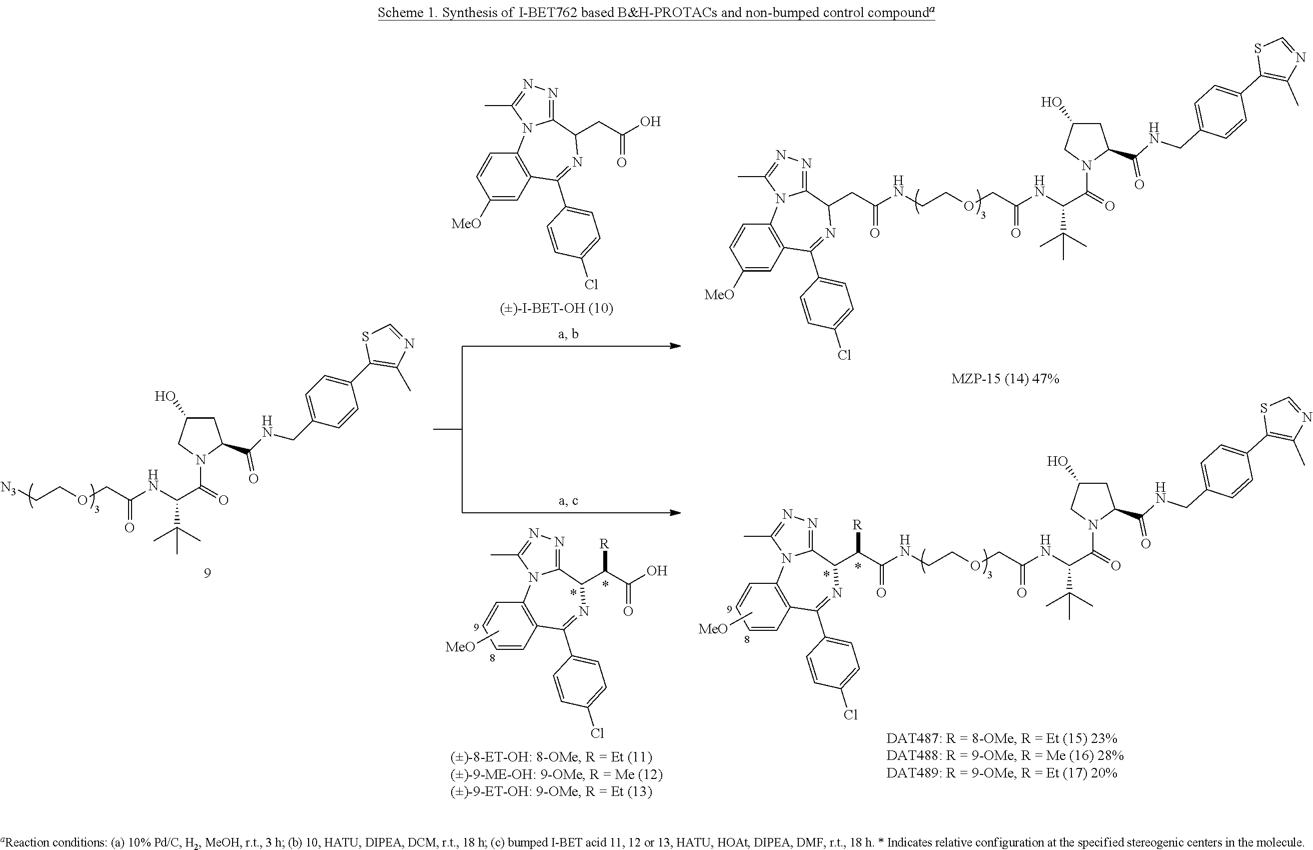

With this initial library in hand, we set out to evaluate the activity and selectivity of our I-BET-based B&H-PROTACs by treating our heterozygous BromoTag-Brd2 HEK293 cells with 1 μM of compounds 15-17 or 1 μM of control compounds MZP-15 (14), MZ1 (1) and cis-MZ1 for 6 h, which is sufficient time to achieve effective MZ1-induced BET protein degradation (FIG. 4A). Cells were harvested, and the subsequent lysate was analysed via western blot using antibodies against BET proteins; Brd2, Brd3 and Brd4 (FIG. 4A). To our disappointment, none of the initial B&H-PROTAC compounds induced detectable degradation of the BromoTag-Brd2.

In understanding the potential reasons for the inactivity of our initial set of compounds, we were curious to observe the apparent significant lower activity of non-bumped 14 relative to 1 across all three BET family members (FIG. 4A). While its presence as diastereomeric mixture may contribute to the lower apparent activity of 14 compared to enantiomerically pure 1, we turned our attention to the chemical structures of the two compounds. Compounds 1 and 14 are otherwise structurally identical except that they differ in the BET bromodomain binding portion: 1 (JQ1-based) bears a dimethylthiophene group fused to the diazepine ring, while 14 (I-BET762-based) bears an 8-OMe-phenyl group in the equivalent position (c.f. FIG. 1B and Scheme 1). We therefore turned to our structural superposition between the ternary crystal structure Brd4^{BD2}:1:VHL and the binary structures of the bumped BET ligands and inspected in more detail the region of the structures around the different groups. This analysis revealed that the methoxy group of the fused phenyl ring present in I-BET762-derived ligands would clash with His110 present in VHL (FIG. 4B). The oxygen atom of the 8-OMe of 6 would be ˜3.0 Å away from the carbon atom between the two nitrogen atoms of the His110 side chain, which is below the lower limit for Van der Waal's interactions.

We reasoned that such structural clash would destabilise the MZ1-like PROTAC ternary complex, explaining the lower degradation activity of the I-BET762-based compounds. This observation led to the decision to replace the 8-OMe-phenyl group in the BET binding portion of the PROTACs with the dimethyl-thiophene group, to develop compounds much more closely resembling the full chemical structure of MZ1 as a design strategy to minimize any potential disruption in the desired ternary complex, and enhance BromoTag degradation activity.

Development of second generation, JQ1-based, B&H-PROTACs. To overcome the limitations presented by our I-BET762-based B&H-PROTACs, we next designed a new set of eight JQ1-based compounds (Table 1). Around this time in the development of the project, we learnt of another BET targeting PROTAC, ARV-771 (2, FIG. 1A), that is structurally similar to 1 and also potently degrades the BET proteins (Raina, K. et al., Proc Natl Acad Sci USA. 2016, 113 (26), 7124-7129). Chemically, 2 consists of the same pan-selective BET bromodomain ligand, (+)-JQ1 (3, FIG. 1B), but differs from 1 by having a shorter, more lipophilic linker (minus —CH₂O—) and an extra benzylic methyl group in the VHL ligand VH032 (Galdeano, C. et al., J Med Chem. 2014, 57 (20), 8657-8663), which is known to boost VHL binding affinity (Hu, J. et al., J Med Chem. 2019, 62 (3), 1420-1442). To maximise chemical diversity and hence our chance to identify a potent BromoTag degrader, we designed four bumped PROTAC compounds based on 1 and four based on 2 (Raina, K., 2016 (supra)). For each set of four, two compounds would contain either a more sterically conserved methyl bump or a more sterically demanding ethyl bump. Each methyl or ethyl bumped BET bromodomain ligand would then be conjugated to the linker via an amide bond to resemble the parent compound or via an ester bond. Our reasoning for choosing the less conventional ester conjugation was based on our previous observation that bumped BET ligands bearing an ester group adjacent to the alkyl bump group were significantly more stable compared to their parent non-bumped analogues (Runcie, A. C., 2018 (supra)).

TABLE 1
Target JQ1 based B&H-PROTAC library

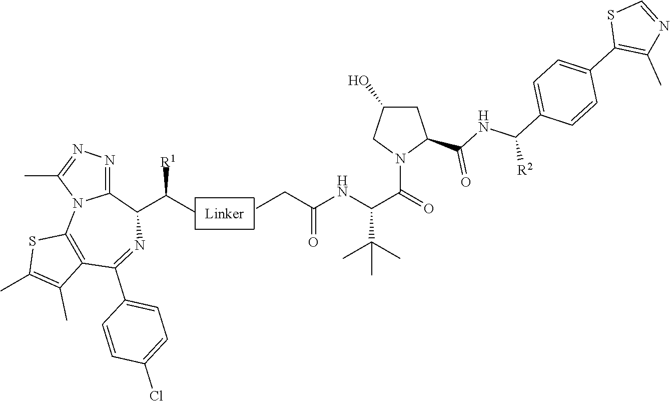

| Compound | Linker | R¹ | R² |
|---|---|---|---|
| ME-MZ1 (18) | 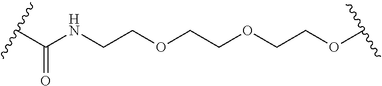 [SMILES: *OCCOCCOCCNC(*)=O] | Me | H |
| ET-MZ1 (19) | 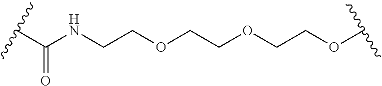 [SMILES: *OCCOCCOCCNC(*)=O] | Et | H |
| ME-ARV-771 (20) | 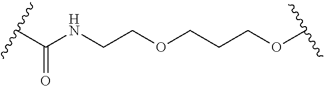 [SMILES: *OCCCOCCNC(*)=O] | Me | Me |
| ET-ARV-771 (21) | 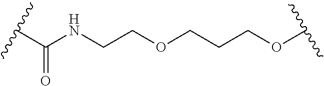 [SMILES: *OCCCOCCNC(*)=O] | Et | Me |
| ME-OMZ1 (22) | 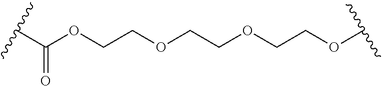 [SMILES: *OCCOCCOCCOC(*)=O] | Me | H |
| ET-OMZ1 (23) | 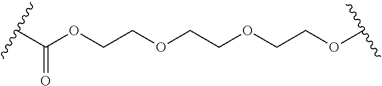 [SMILES: *OCCOCCOCCOC(*)=O] | Et | H |
| ME-OARV-771 (24) | 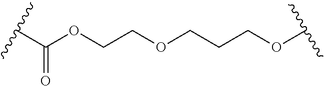 [SMILES: *OCCCOCCOC(*)=O] | Me | Me |
| ET-OARV-771 (25) | 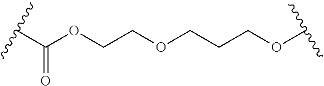 [SMILES: *OCCCOCCOC(*)=O] | Et | Me |

To synthesise our bumped-JQ1 ligands, we adapted the route described by Filippakopoulos, P et al. in Nature 2010, 468, 1067-73 and utilised the late stage alkylation described by Baud, M. G. J., 2014 (supra) and Runcie, A. C., 2018 (supra) (Scheme 2). Firstly, (±)-Fmoc-Asp(OMe)-OH (26) was treated with thionyl chloride in dichloromethane (DCM) and converted to the acid chloride before being refluxed with aminoketone 27 in chloroform to form an ‘open’ amide Fmoc-protected intermediate. This ‘opened’ intermediate is then refluxed in triethylamine to remove the Fmoc protecting group and reveal the free amine, which in the presence of acetic acid, ring closes to form the thieno-1,4-diazepine, 28. Deprotonation of amide 28 with potassium tert-butoxide in the presence of diethyl chlorophosphate followed by treatment acetylhydrazine forms the methyltriazole ring and yields triazolothienodiazepine (±)-JQ1-OMe (29) as a racemic mixture.

To introduce either methyl or ethyl bump, 29 was deprotonated with potassium hexamethyldisilazane (KHMDS) at −78° C. in tetrahydrofuran (THF). The subsequent enolate was then treated with either methyl or ethyl iodide to yield racemic bumped-JQ1-OMe derivatives 30a and 30b or 31a and 31b, respectively as mixtures of diastereomers which were easily separated following HPLC. Methylation proceeded with a diastereomeric ratio (d.r.) of 1:4 for the desired (2S*,3R*) isomer to the undesired (2S*,3S*) isomer. Ethylation proceeded with a d.r. of 1:1.5. The undesired (2S*,3S*) isomers, 30b and 31b, can be epimerised by treating with sodium methoxide in methanol under microwave irradiation to yield a further 1:1 mixture of diastereomers, which, following HPLC separation, yields more of the desired (2S*,3R*) isomers 30a and 31a.

To allow for further functionalisation and linker conjugation, methyl esters 30a and 30b were hydrolysed under mild conditions with lithium hydroxide in THF and water to yield the conjugatable carboxylic acids 32 and 33 as racemic mixtures (Scheme 2).

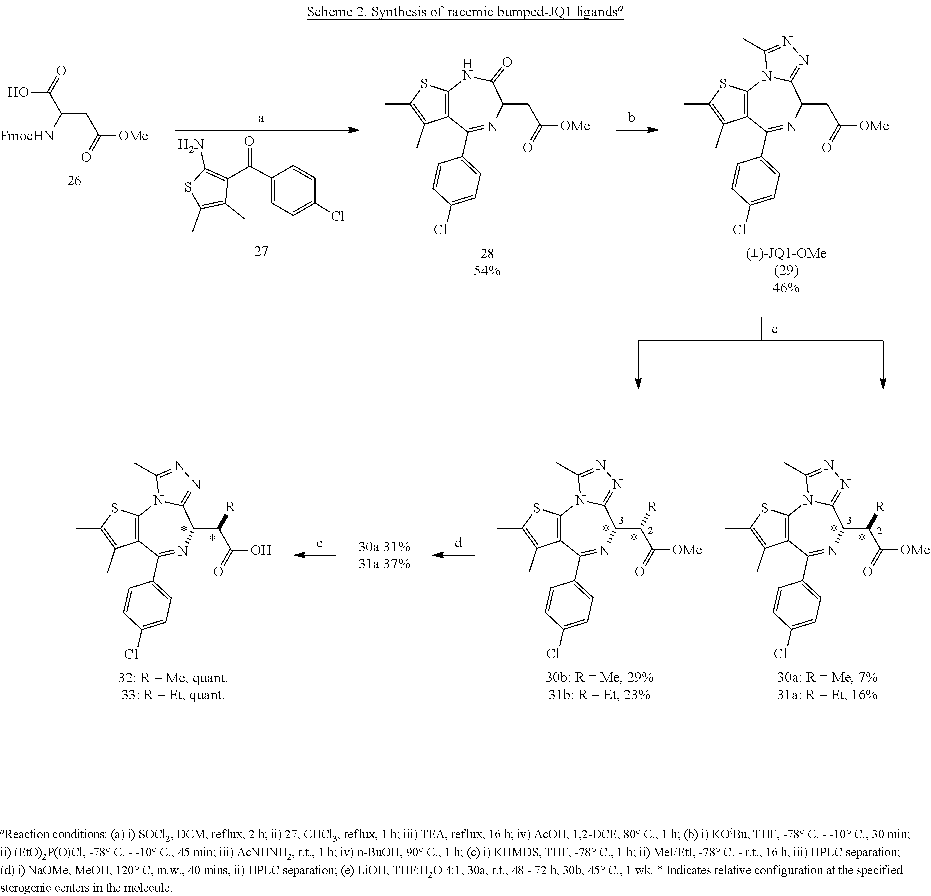

Next, was to connect linkers, 36 and 37, to VH032-amine, 34, and linkers, 38 and 39, to methylated VH032-amine, 35, via standard amide coupling conditions with HATU and DIPEA in DMF to yield amides, 9, 40, 42, and 43 (Scheme 3). Silylethers, 40 and 43, were cleaved using a solution of tetrabutylammonium fluoride (TBAF) in THF to yield terminal alcohols 41 and 44, respectively, as suitable precursors for ester conjugation.

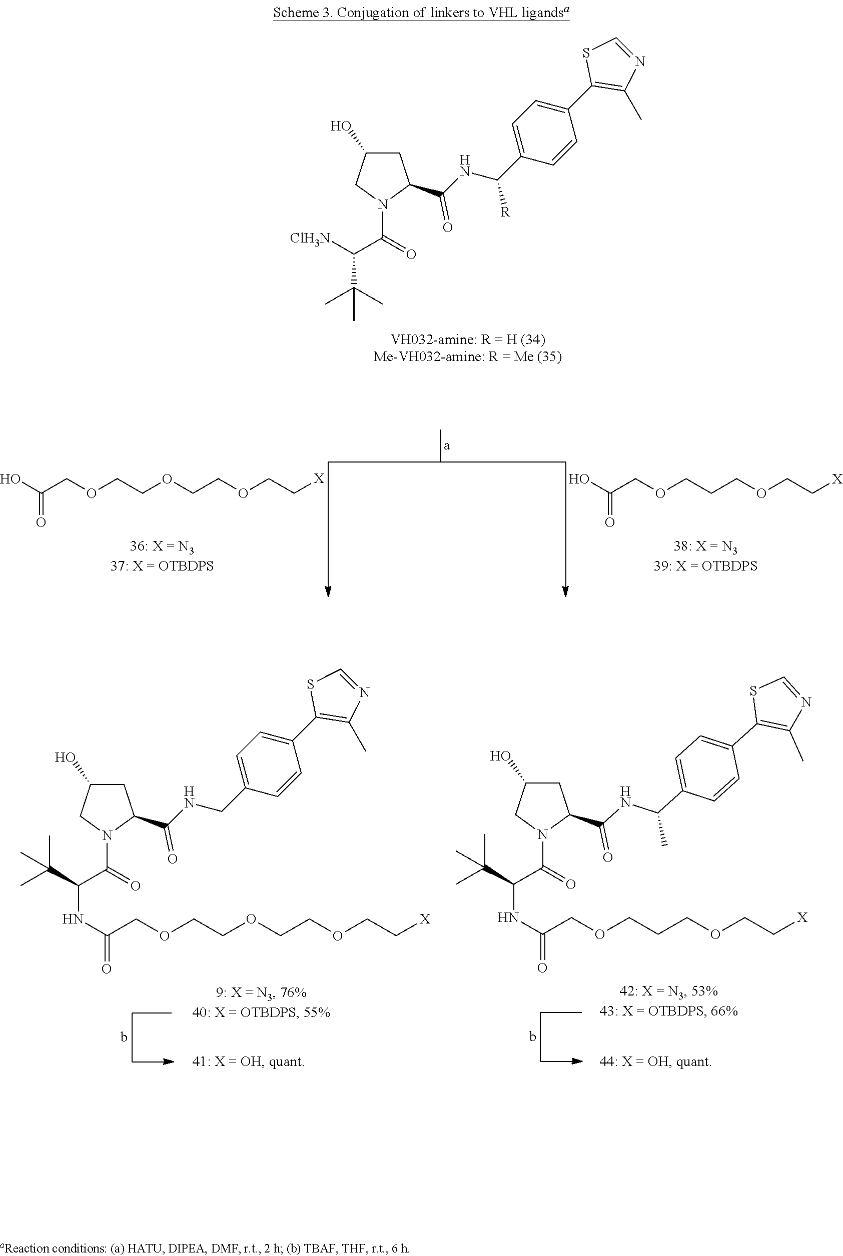

Azides, 9 and 42, were subsequently reduced with a suspension of 10% palladium on carbon in methanol under an atmosphere of hydrogen gas to yield terminal amines before being coupled to racemic bumped-JQ1 acids, 32 and 33, using (1-cyano-2-ethoxy-2-oxoethylidenaminooxy)dimethylamino-morpholino-carbenium hexafluorophosphate (COMU) and DIPEA in THF to yield amide B&H-PROTACs, 18-21 as a mixture of diastereomers (Scheme 4).

Finally, alcohols 41 and 44 were coupled to bumped-JQ1 acids, 32 and 33, with N-(3-dimethylaminopropyl)-N′-ethylcarbodiimide hydrochloride (EDC·HCl) and 4-(dimethylamino)pyridine (DMAP) in THF to yield ester B&H-PROTACs, 22-25, as a mixture of two diastereomers (Scheme 4). The diastereomers formed in each amide and ester case were inseparable by HPLC and were progressed as diastereomeric mixtures to preliminary in cellulo evaluation to screen for BromoTag-Brd2 degradation and selectivity over wild-type BET proteins.

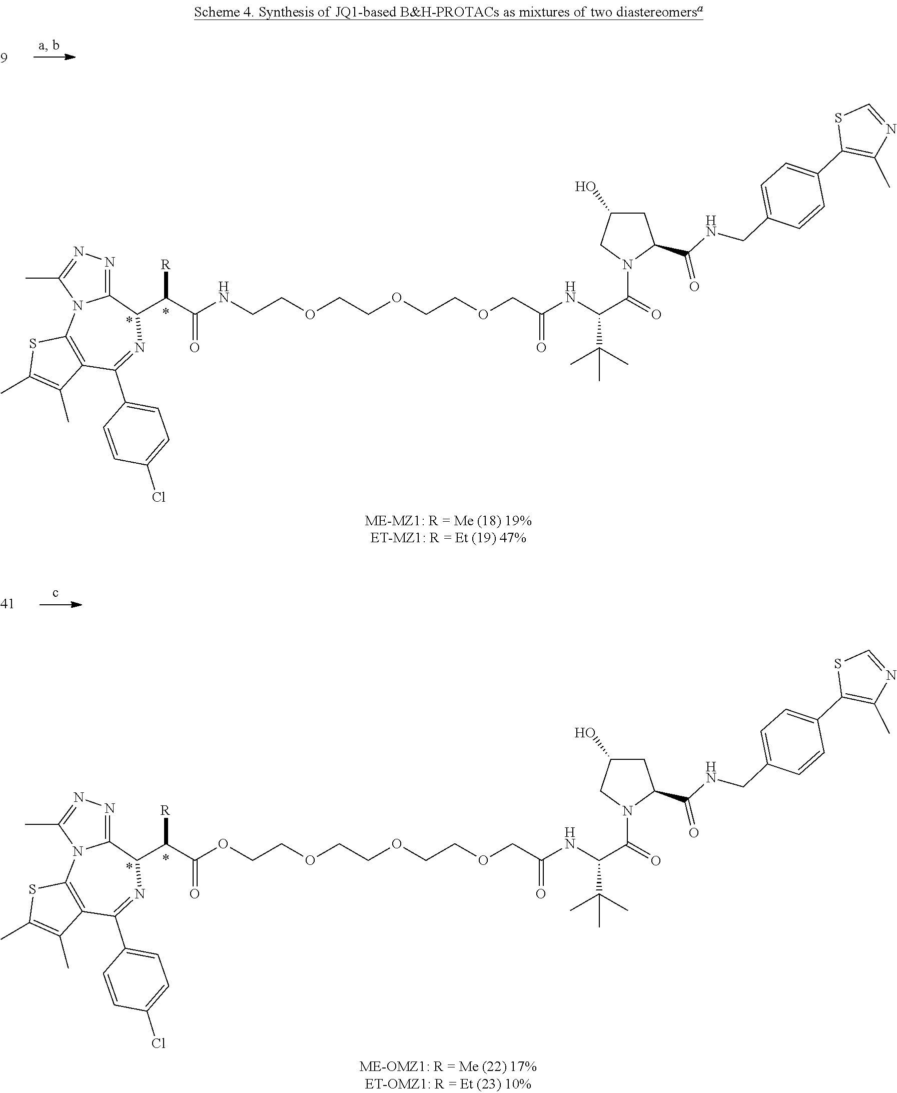

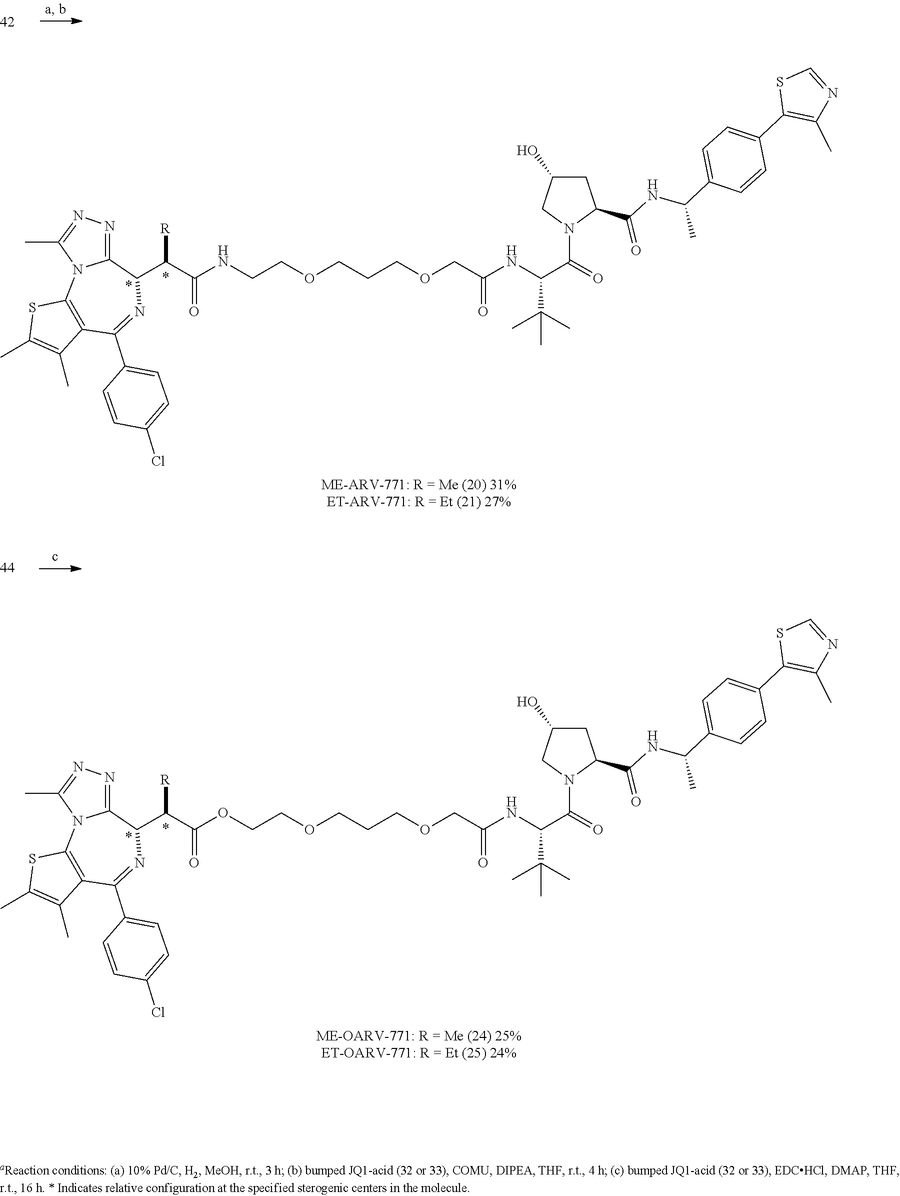

We next evaluated the cellular activity of all eight B&H-PROTACs (18-25) in our heterozygous BromoTag-Brd2 knock-in HEK293 cell line at concentrations ranging from 1 nM to 10 μM (FIG. 5). Strikingly, all compounds showed a pronounced effect on the degradation of the BromoTag-Brd2 isoform, achieving observable and in most cases complete depletion of BromoTagged-Brd2 protein. This allowed quantitative analysis of the compounds' on-target degradation potency (DC₅₀) and efficacy (Dₘₐₓ) to build SAR, with quantification performed via detection using both independent antibodies, which compared extremely well in all cases (Table 2).

The best compounds emerged to be 19, 23 and 25, which all harbour an ethyl bump. They showed both potent and complete degradation of the BromoTag-Brd2 isoform, with DC₅₀ values of 250-360 nM, 13-80 nM, and 13-16 nM, and Dₘₐₓ>75%. Importantly, no observable off-target degradation of the untagged Brd2 or the other endogenous BET proteins was observed, except minor off-target degradation of Brd3 observed with 25 at 1 μM (FIG. 5), suggesting these compounds successfully enable highly selective BromoTag degradation. Interestingly, at the higher concentrations used of 1-10 μM (FIG. 5) esters 23 and 25 showed strong onset of the hook effect, a well-known phenomenon with bifunctional PROTAC degraders where binary interactions between PROTAC:target and PROTAC:E3 ligase outcompete productive ternary complex formation. In contrast, no hook effect was observed with the amide 19. The last ethyl-bumped compound, 21, showed the least complete (Dₘₐₓ<70%) and weakest (DC₅₀˜1 μM) degradation activity, with a very narrow degradation window also due to strong hook effect at 10 μM.

All methyl bumped compounds 18, 20, 22 and 24 also showed strong on-target degradation activity and were on average 2-fold more potent than their ethyl bumped counterparts, with DC₅₀s between 100-160 nM, 320-400 nM, 20-80 nM, and 5-10 nM respectively. However, we observed, all methyl bumped compounds also induced undesired off-target degradation, thus showing poor selectivity. These results suggest that the methyl group does not provide enough of a steric clash with the conserved Leu residue of the wild-type BET proteins that is not sufficient to dial out off-target degradation and is much more tolerated than the larger ethyl bump. Since selectivity against endogenous BET protein is a strictly required criterion for a successful BromoTag system, we decided to drop all methyl bumped compounds at this stage.

It is interesting to note that all esters (22-25) are more potent than their respective amide counterparts (18-21) by between 2- and 126-fold in DC₅₀ (Table 2). Recently, we have shown that the amide-to-ester substitution can provide a simple strategy to increase PROTAC degradation activity due to increased lipophilicity and cellular permeability, while maintaining remarkable intracellular stability (Klein, V. et al. 2021(supra)). This trend is well reflected in this compound set, as all esters (22-25) were cell-active and showed more potent DC₅₀ and a prominent hook-effect at the higher concentrations compared to their amide counterparts.

TABLE 2
Quantification of the degradation profile of
second-generation B&H-PROTACs against BromoTagged-
Brd2 via two different antibodies (Ab).

| | Ab: Brd4^{BD2 L387A} | | Ab: Brd2 | |
|---|---|---|---|---|
| Compound | pDC₅₀^{a} | Dₘₐₓ (%) | pDC₅₀^{a} | Dₘₐₓ (%) |
| ME-MZ1 (18) | 6.8 ± 0.1 | 90 | 7.0 ± 0.2 | 92 |
| ET-MZ1 (19) | 6.6 ± 0.6 | 76 | 6.5 ± 0.2 | 80 |
| ME-ARV-771 (20) | 6.5 ± 0.4 | 70 | 6.4 ± 0.6 | 60 |
| ET-ARV-771 (21) | 6.1 ± 0.3 | 69 | 5.7 ± 1.8 | 51 |
| ME-OMZ1 (22) | 7.1 ± NA | 94 | 7.7 ± 0.1 | 96 |
| ET-OMZ1 (23) | 7.1 ± NA | 90 | 7.9 ± 0.2 | 96 |
| ME-OARV-771 (24) | 8.0 ± NA | 91 | 8.3 ± 0.1 | 91 |
| ET-OARV-771 (25) | 7.9 ± 0.2 | 90 | 7.8 ± 0.3 | 85 |

^{a}calculated as mean (±S.E) from three independent biological experiments.

Taken together, the results from this screen identified three compounds, ET-MZ1 (19), ET-OMZ1 (23) and ET-OARV-771 (25) as the most selective BromoTag-Brd2 degraders, meeting criteria for potent on-target activity while largely sparing off-target BET degradation. We therefore took these three compounds forward in the pipeline.

Synthesis of ET-OMZ1, ET-OARV-771 and ET-MZ1 as single stereoisomers. We realised that our second-generation B&H-PROTACs, while displaying encouraging results, were all synthesized as diastereomeric mixtures, meaning they would not only contain the active species but also an inactive or less-active species that would be expected to lead to apparent weaker activity and narrower selectivity window of the compounds. To gain a true degradation profile of the biologically active isomers (eutomers), we therefore next sought to synthesise our current best degraders as enantiomerically pure, single diastereomers. To achieve enantiomerically pure PROTACs, we developed a new stereoselective synthesis to bumped BET ligands, which we disclosed recently Bond, A. G., 2020, (supra). In brief, our novel stereoselective route allowed us to incorporate the alkyl bump much earlier in the synthesis of the BET-ligand scaffold. To achieve this, we optimized a lithium hexamethyldisilazane (LHMDS)-mediated, diastereoselective alkylation of a di-protected aspartate derivative, and took this through to final bumped-JQ1-acid analogues with complete retention of stereochemistry and in >99% ee (Bond, A. G., 2020, (supra). At this stage in the project, we therefore decided to use the enantiomerically pure ET-JQ1-OH (45) to make new B&H-PROTACs, AGB1 (46), AGB2 (47) and AGB3 (48) (Scheme 5).

For esters 46 and 47, acid 45 was converted quantitatively to an acid chloride intermediate with thionyl chloride in DCM and was subsequently reacted with alcohols 41 and 44 to yield final compounds 46 and 47 as single stereoisomers. For amide 48, azide 9 was first reduced with a suspension of 10% palladium on carbon in methanol under an atmosphere of hydrogen gas to yield the intermediate amine which was immediately coupled to 45 using COMU and DIPEA in DMF to yield 48 as a single stereoisomer.

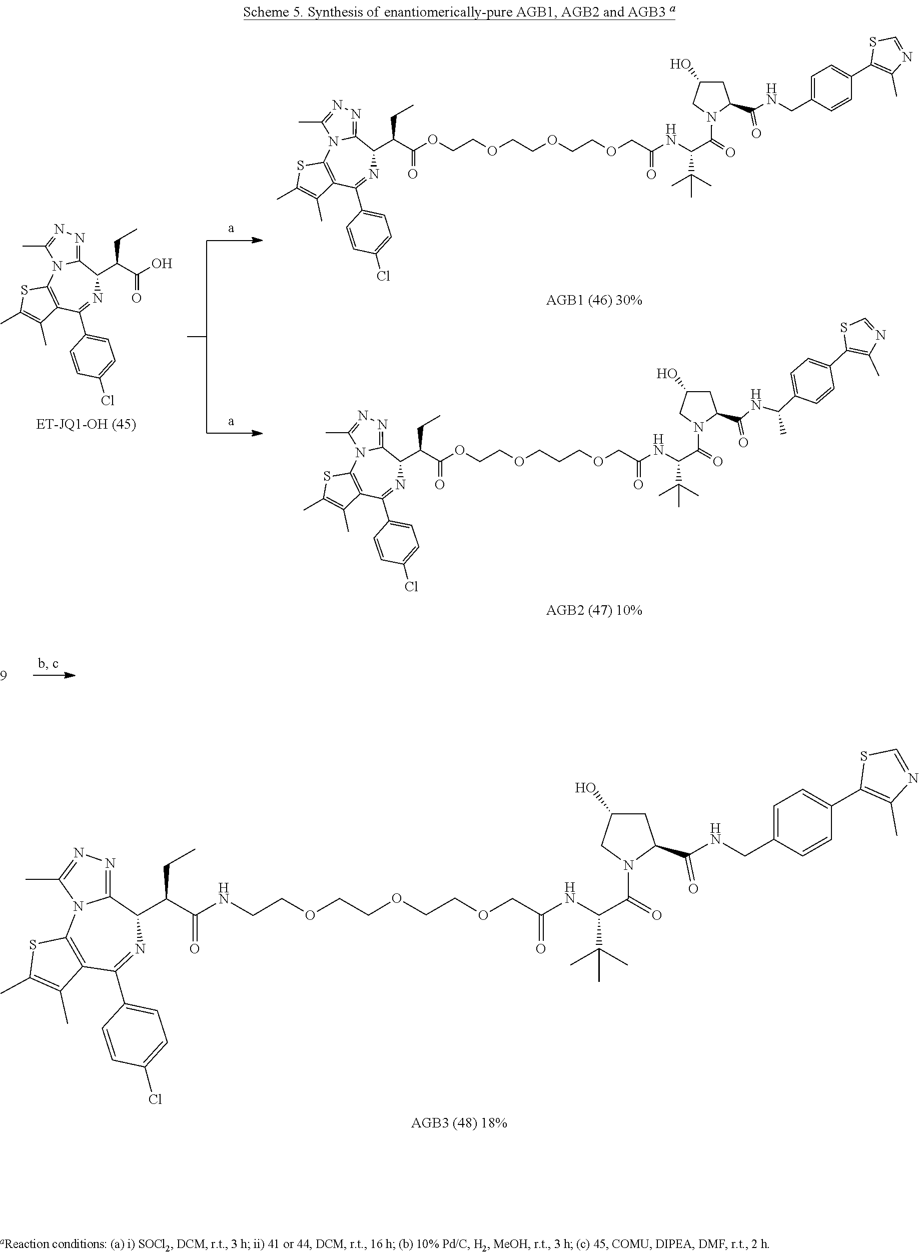

We next evaluated the cellular activity of 46-48 using our BromoTag-Brd2 knock-in cell line as described before (FIGS. 6A & C, Table 3). At this stage we decided to quantify protein degradation over a wider 8-point, concentration range from 10 μM-1 nM for the purpose of obtaining more accurate DC₅₀ and Dₘₐₓ values. Each compound displayed potent, highly selective and near complete (Dₘₐₓ>92%) degradation of BromoTag-Brd2 over the endogenous BET proteins. Both ester 46 (DC₅₀ 13-15 nM) and 47 (DC₅₀ 1-3 nM) showed more potent on-target degradation activity than amide 48 (DC₅₀ 210-290 nM), corresponding to >17-fold and >81-fold lower DC₅₀, respectively (FIGS. 6A & C, Table 3). As predicted, the enantiomerically-pure compounds were found to be on average 5-fold more potent, and displayed much more complete on-target degradation, than when they were tested as diastereomeric mixtures (c.f. 23, 25 and 19 respectively, FIG. 5, Table 2). The large difference in potency displayed by the esters is exemplified by the pronounced hook effect at concentrations >1 μM. From our recent work on amide-to-ester substitutions in related non-bumped BET PROTACs, the increase in potency observed is most likely due to the increase in cellular permeability as a result of increased lipophilicity from switching from an amide in 48 to an ester in 46 (Klein, V. et al. 2021(supra)).

TABLE 3
Degradation profile for AGB1, AGB2 and AGB3

| | Ab: Brd4^{BD2 L387A} | | Ab: BromoTag-Brd2 | | |
|---|---|---|---|---|---|
| Compound | pDC₅₀^{a} | Dₘₐₓ (%) | pDC₅₀^{a} | Dₘₐₓ (%) | t_{1/2} (min) ^{b} |
| AGB1 (46) | 7.8 ± 0.2 | 99 | 7.9 ± 0.1 | 97 | 40 ± 15 |
| AGB2 (47) | 9.0 ± 0.4 | 100 | 8.6 ± 0.2 | 98 | 142 ± 13 |
| AGB3 (48) | 6.5 ± 0.3 | 92 | 6.7 ± 0.1 | 96 | 113 ± 8 |

^{a}Calculated from mean (±S.E.) of two independent repeats
^{b} Calculated from mean (±span) of two independent repeats

To assess the speed at which our B&H-PROTACs were able to fully deplete BromoTag-Brd2, we next ran a time-dependent degradation assay by treating heterozygous BromoTag-Brd2 HEK293 cells with 500 nM of 46 or 47, or 1 μM of 48, and measuring BromoTag-Brd2 protein levels over 36 h (FIGS. 6B & D, Table 3). Compound 46 proved to be the fasted and most complete degrader, able to completely degrade BromoTag-Brd2 within 6 h, yielding a protein half-life (t_{1/2}) of just 40 min. Compound 48 was a slightly slower degrader, inducing a protein t_{1/2} of 113 min, and was only able to degrade up to ˜80% of the protein in this experiment (FIG. 6D). That is, despite 48 being used at twice the treatment concentration of 46, clearly demonstrating the more potent, faster and more profound activity of the ester bumped PROTAC. The other ester compound 47 showed near complete target degradation, similar to 46, but not as fast (t_{1/2}=142 min) when compared with 46, which combined to residual albeit minor off-target degradation of Brd3 at 100-1,000 nM (FIG. 6), led us to deprioritize 47. Together, the cellular data suggest 46 as the best degrader amongst the three evaluated as single stereoisomers.

To better understand the mode of action of our three B&H-PROTACs, we next sought to investigate the ability of each compound to form ternary complex between recombinantly-purified Brd4^{BD2 L387A} bromodomain protein and VHL. We therefore employed a competitive fluorescence polarization (FP) assay as previously published (Roy, M. J., 2019 (supra), where we displace a fluorescently labelled HIF-1a peptide probe bound to VHL by titrating either compound alone (for binary binding) or by titrating compound preincubated with Brd4^{BD2} L^{387A} protein (for ternary complex binding). Cooperativity (a) of ternary complex formation can then be calculated (α=K_{d}^{binary}/K_{d}^{ternary},) (FIG. 7). A ternary complex is said to have positive, or negative cooperativity when α>1 or α<1 respectively, and not cooperative when α=1. PROTACs 46, 47 and 48 had equipotent ternary binding affinity (K_{d}=11 nM, 12 nM and 9 nM, respectively), with MZ1-based 46 and 48 giving the most cooperative ternary complexes (α=11.1 and 10.9 respectively). ARV-771-based 47 formed the least cooperative ternary complex (α=3.6, FIG. 7B) due to its 2-3 fold greater binary affinity to VHL (K_{d}=45 nM for 47, compared to K_{d}=125 nM and 102 nM for 46 and 48, respectively). This loss in ternary complex cooperativity and stability is likely contributing to the slower rates of degradation observed, consistent with previous findings with BET PROTACs (Roy, M. J., 2019 (supra).

With all this biological data taken together, we selected 46 as our best B&H-PROTAC and decided to take this forward for further biological evaluation.

Further biological and mechanistic characterisation of AGB1. Having established AGB1 (46) as the best potent and selective degrader compound for our BromoTag system, we next sought to further characterize its mechanism of action as expected for this compound class. To demonstrate that the on-target degradation activity of 46 is mechanistically as expected due to its PROTAC mode of action, we performed pharmacological competition experiments (FIG. 8A). To demonstrate VHL- and proteasome-dependency, we pre-treated with the NAE1 inhibitor MLN4924, which inhibits neddylation of Cullin 2, with the VHL inhibitor VH298, and with the proteasome inhibitor MG132. In addition, to demonstrate that the on-target activity on BromoTag-Brd2 is due to recruitment of the BromoTag, we pre-treated with ET-JQ1-OMe which binds with high affinity to the Brd4^{BD2 L387A} variant (K_{d}=65 nM) but exhibits undetectable binding to the wild-type domain (Bond, A. G., 2020, (supra). In this experiment, we separately exposed our heterozygous BromoTag-Brd2 HEK293 cells to the different inhibitors for a short 1 h prior to subsequent treatment with 200 nM 46 before continuing treatment for a further 3 h, to minimize potential confounding effects due to inhibitors cytotoxicity. As expected, the on-target degradation activity by 46 was completely abrogated upon pre-treatments with MLN4924 or VH298, demonstrating dependency on CRL2^{VHL} activity (FIG. 8A). The cellular activity of MLN4924 and VH298 in this experiment was confirmed by observing significant accumulation of HIF-1a, and blockade of Cul2 neddylation upon MLN4924 treatment. The on-target degradation activity was shown to be proteasome-dependent as it was blocked upon pre-treatment with MG132. A similar outcome was also seen from the pre-treatment with ET-JQ1-OMe (FIG. 8A), confirming the on-target degradation is exquisitely driven by target engagement with the BromoTag, and not contributed by potentially adventitious weaker recruitment of the wild-type bromodomains of the endogenous Brd2 protein.

We next sought to monitor the duration of the on-target degradation activity of 46 using washout experiments. BromoTag-Brd2 HEK293 cells were treated with 200 nM 46 for 3 h, rinsed twice with phosphate-buffered saline (PBS), and fresh media was replenished without PROTAC. Following complete depletion after 3 h, protein levels of BromoTag-Brd2 were shown to recover 24 h after washout (FIG. 8B). This effect was in stark contrast to the complete and durable on-target degradation for up to 72 h without washout. This result confirms the reversible nature of our BromoTag system. Noticeably, Brd2 expression begins to decline 24 h post recovery, possibly reflecting long-term regulation of Brd2 protein levels.

To qualify our degrader 46 as suitable chemical probe for cellular biological investigation, we considered it important to assess potential cytotoxicity that might confound biological effects and responses and mask the desired on-target pharmacology. To this end, we elected as probe criteria that the compound does not exhibit any cytotoxicity at around and up to 10-fold higher than the concentrations at which it is to be used in cells. The remarkable selectivity and lack of off target BET degradation activity of 46 encouraged us that the compound should not be cytotoxic, but we decided to test this in parent HEK293 cells, as well as more BET-sensitive MV-4-11 and 22RV1 cell lines. To enable a suitable control to discount any potential non-degrading off-target engagement activity, we synthesized compound cis-AGB1 (52) bearing the cis- instead of trans-hydroxyproline group to abrogate binding to VHL (Scheme 6), a well-established strategy to yield negative non-degrading control compounds (Zengerle, M, 2015, (supra)). To monitor cell viability, HEK293, MV-4-11 & 22RV1 cells were plated in a 96-well plate format and treated with vehicle control (DMSO), 46, its non-degrading control (52), and their non-bumped control compounds MZ1 and cis-MZ1, as well as the positive control cytotoxic agent staurosporine, in a dose-dependent manner and up to 10 μM. Cellular ATP levels as a proxy of viable cells was then measured using Cell Titre Glo® 2.0 luminescent reagent (FIG. 8C). Reassuringly, and as expected, 46 showed lack of cytotoxicity up to the high concentrations used of 1-10 μM, in all three cell lines. The remarkable sparing of off-target degradation of endogenous BET proteins by 46 is starkly evidenced by comparing it with MZ1 (EC₅₀ of ˜20 nM) in the highly BET-sensitive MV-4-11 cells. Together this data qualifies 46 as mechanistically clean and bona-fide BromoTag degrader for cellular investigation.

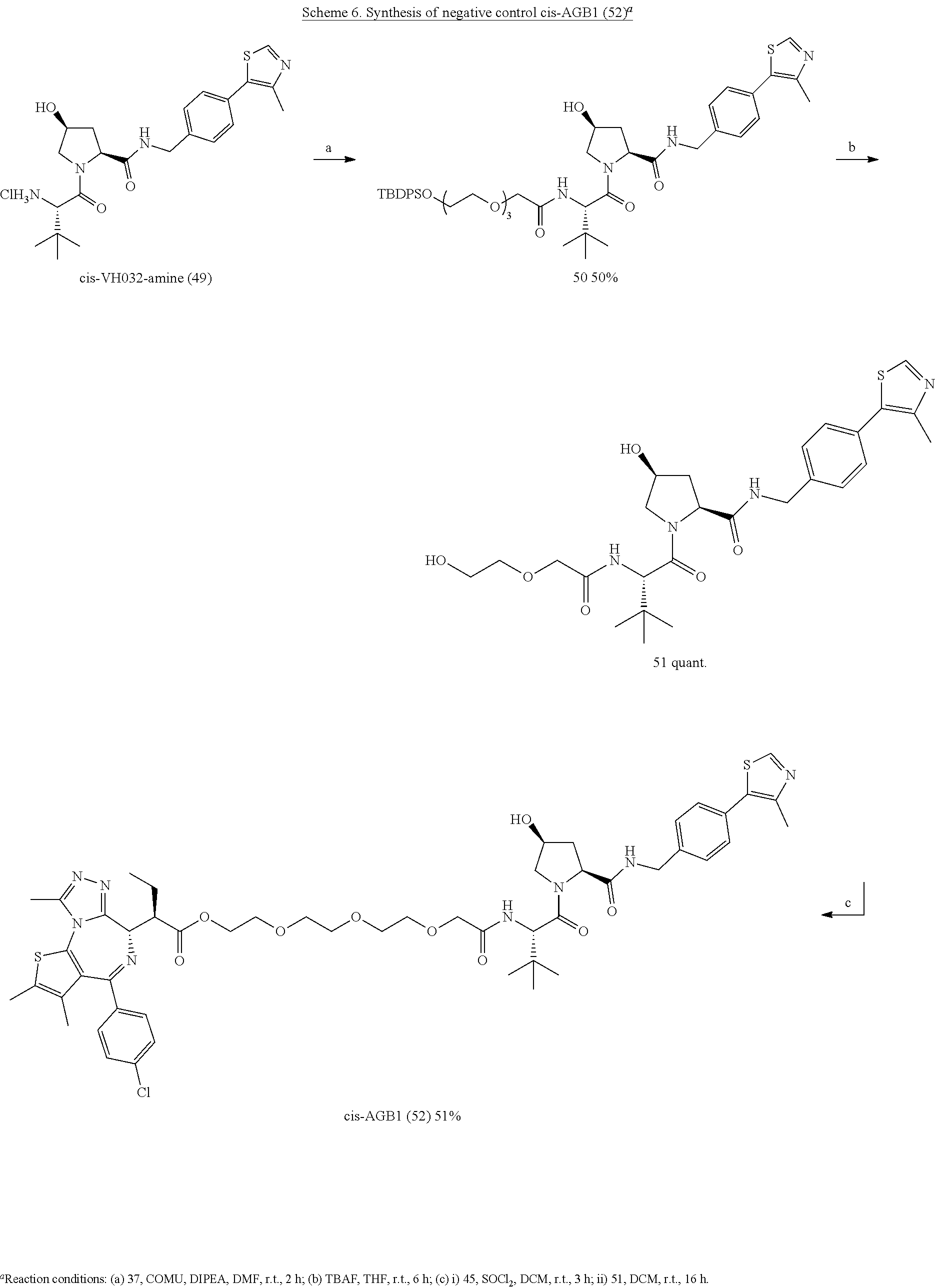

We next evaluated the plasma stability of 46 by incubating in mouse plasma at 37° C. and measuring the levels of 46 remaining at several time points over 1 h. Compound 46 showed excellent plasma stability with no significant changes to levels of 46 throughout the experiment. Finally, to further qualify 46 as appropriate for in vivo studies, we next assess its pharmacokinetic (PK) profile in mice (FIG. 9, Table 4 & 5). 46 was shown to have good PK profiles in mice for both intravenous (IV) (Table 4) and subcutaneous (SC) (Table 5) 5 mg/kg injections. 46 has comparable PK profiles as seen for parent compound MZ1 (1) with a relatively low clearance rate (CL) of 47.2 mL/min/kg and good half-lives (T_{1/2}) of 1.49 h and 1.65 h in IV and SC respectively (compared with 1.05 h and 2.95 h for 1) (Table 4 & 5) (Boehringer Ingelheim—opnMe. https://opnme.com/molecules/bet-mz-1 (accessed 24/08/2021). Strikingly, 46 was able to maintain a plasma concentration above its BromoTag-Brd2 DC_{50, 6 h} of <15 nM when dosed at 5 mg/kg for −4 h IV injection and for >8 h SC injection (FIG. 9), making it suitable for in vivo studies to assess the functional consequences of BromoTagged target protein degradation in genetically engineered mouse models.

TABLE 4
Pharmacokinetic Study of AGB1 in Mice with IV Dosing compared to MZ1

| | IV (5 mg/kg) | | | | | |
|---|---|---|---|---|---|---|
| | CL | | | AUCₗₐₛₜ | AUC_{inf} | |
| Compound | (mL/min/kg) | Vₛₛ (L/kg) | T_{1/2} (h) | (μM · h) | (μM · h) | MRT_{inf} (h) |
| AGB1 (46) | 47.2 | 1.10 | 1.49 | 1.71 | 1.72 | 0.390 |
| MZ1 (1)^{a} | 19.7 | 0.38 | 1.04 | — | 4.51 | 0.340 |

^{a}values obtained from ref.²⁸

TABLE 5
Pharmacokinetic Study of AGB1 in Mice
with SC Dosing compared to MZ1

| | SC (5 mg/kg) | | | | | |
|---|---|---|---|---|---|---|
| | | | | AUCₗₐₛₜ | AUC_{inf} | |
| | | | | (μM · | (μM · | |
| Compound | Tₘₐₓ (h) | Cₘₐₓ (μM) | T_{1/2} (h) | h) | h) | F (%) |
| AGB1 (46) | 0.500 | 0.700 | 1.65 | 2.33 | 2.40 | 140 |
| MZ1 (1)^{a} | 0.500 | 2.07 | 2.95 | — | 3.76 | 83 |

^{a}values obtained from ref.²⁸

Degradation of a variety of target proteins. We employed the techniques as described above, in order to demonstrate the generic applicability of the described process to the degradation of a variety of target proteins. We performed CRISPR knock-in of the BromoTag, as described, into the endogenous loci of five additional proteins. The results are shown in Table 6 below, where it can be seen that CRISPR knock-in of the BromoTag demonstrates a broad scope of proteins that are targetable by the BromoTag degron system. The target proteins of Table 6 are all different, are all known to have different cellular roles and function, intracellular expression, and distinctive turnover rates within cells.

TABLE 6
Degradation profile of five different target proteins using AGB1

| | | AGB1 | | |
|---|---|---|---|---|
| BromoTag'ed Target Protein | Tag | pDC₅₀ | Dₘₐₓ (%) | t_{1/2} (min) |
| BRD2 (P25440) | N-term | 7.8 | 97 | 40 |
| BRD4 (O60885) | N-term | 7.9 | >95 | n.d. |
| MCM2 (P49736) | N-term | 8.0 | >95 | n.d. |
| MCM4 (P33991) | N-term | 8.1 | >99 | 13 |
| PPM1H (Q9ULR3) | C-term | >7.0 | >95 | 30 |
| VPS34/PK3C3 (Q8NEB9) | N-term | 8.0 | >99 | n.d. |

As seen in the table above, in all instances where tagging has been successful, we have been able to induce degradation of our BromoTag conjugates using our PROTAC compound AGB1 in cells. These results also indicate that our tag can induce degradation of the BromoTag in both N- and C-terminal loci. We also demonstrate that we can cause degradation of our BromoTag-protein conjugates to greater than 95% loss of total target protein within a treatment window of 100 nM-1 nM. For those targets tested, we show that AGB1 can produce a half-life degradation of between 13-40 minutes.

To assess the proteome-wide cellular selectivity of AGB1 for its Bromo-tagged target protein, multiplexed tandem mass tag (TMT) labeling mass spectrometry proteomic experiments were performed to monitor protein levels in a quantitative and unbiased fashion. Bromotag-BRD2 CRISPR knock-in HEK293 cells were treated in triplicate with DMSO, 1 μM AGB1, or 1 μM cis-AGB1 for 2 h. Among the >6,621 proteins quantified, BRD2 was found as most significantly degraded by SIM1, while no detectable depletion was detected for BRD3, BRD4 or indeed any other endogenous protein. No significant changes in BRD2 protein abundance were observed in cells treated with cis-AGB1. The same selectivity pattern was observed with a second Bromotagg'ed target CRISPR knock-in cell line. Together, the data support AGB1 as a degrader with exquisite selectivity for the Bromotag and no detectable off-target effects.

### CONCLUSIONS AND FUTURE PERSPECTIVE

Through careful structural guided design, we have developed AGB1 (46), as our super-fast, highly selective, and potent B&H-PROTAC degrader for our new inducible degron system, BromoTag. We show that AGB1 (46) not only forms a strong, cooperative ternary complex between VHL and the BromoTag (Brd4^{BD2 L387A}) but also completely degrades BromoTagged target proteins with low nanomolar potency and exquisite selectivity over wild-type BET proteins and proteome-wide. We also shown that AGB1 (46) is not cytotoxic in several cancer relevant cell lines, further exemplifying its superior selectivity over off-target endogenous BET proteins. AGB1 (46) has also shown excellent plasma stability and acceptable pharmacokinetics for it to be suitable for later in vivo studies in mouse models. We therefore qualify AGB1 (46) and our new BromoTag system as a useful tool to probe biology. We envisage that BromoTag could also be used in tandem with other inducible degrons such as dTAG, AID or HaloPROTACs, as an orthogonal system to simultaneously deplete more than one protein at once.

As described in the background section, XY-06-007, a compound comprising a “bump” as part of a segment that binds to Brd4 and a CRBN-based ligand, has been developed by R. P. Nowak et al. (2021, supra). Our data suggests that the MZ1-like highly cooperative and stable ternary complex formed by AGB1 with VHL and our BromoTag, underscores its fast and profound tagged-target protein degradation that is more significant with AGB1 than XY-06-007. XY-06-007 and AGB1 differ significantly both in the chemistry (I-BET762 rather than JQ1-based, methyl rather than ethyl bump, respectively) and biology (CRBN- rather than VHL-based, Brd4^{BD}1 L94V tag instead of Brd4^{BD2} L387A, respectively). Therefore, the method described herein and that of Nowak et al. provide two distinct methods to induce degradation of bromodomain-tagged proteins, which add to the growing arsenal of inducible degron technologies available to study the effect and implications of rapid and highly selective degradation of a target protein.

1. A degrader compound of formula (IA) 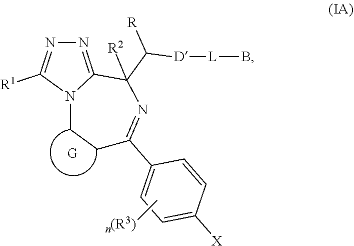
wherein
G is a 5-membered heteroarene optionally substituted with one or two substituents selected from the group consisting of methyl, halo, hydroxy, thiol, halomethyl, amino, methoxy, methylamino, dimethylamino, ethyl, haloethyl, amido, isopropyl, and methylthio, or G is a 6-membered arene or heteroarene optionally substituted with methyl, halo, hydroxy and thiol;
R is C₁₋₄alkyl or C₁₋₄haloalkyl;
R¹ is any one selected from the group consisting of C₁₋₄alkyl, C₁₋₄haloalkyl, H and halo;
R² is H, C₁₋₃alkyl, C₁₋₃haloalkyl or halo;
R³ is independently selected from halo, hydroxyl, thiol, amido, NR⁴R⁵, C(O)NR⁴R⁵, C₁₋₆alkyl, C₁₋₆haloalkyl, C₁₋₆alkoxy and C₁₋₆alkylthio;
R⁴ and R⁵ are independently selected from H and C₁₋₃alkyl;
n is 0, 1, 2, 3 or 4;
X is halo;
D′ is the product of a reactive group, D, with a pro-linker to form D′-L, L is a molecule capable of binding D′ to B and B is a molecule capable of binding to an E3 ubiquitin ligase.

2. The compound of claim 1, wherein G is a 5-membered heteroarene.

3. The compound of claim 1, wherein G is thiophene.

4. The compound of claim 2 wherein G is substituted with:
(i) one or more substituents selected from the group consisting of methyl, halo, hydroxy, thiol, halomethyl, amino, methoxy, methylamino, dimethylamino, ethyl, haloethyl, amido, isopropyl, tert-butyl and methylthio; or
(ii) one or more substituents selected from the group consisting of methyl, halo, hydroxy, thiol, halomethyl and amino.

5. The compound of claim 1, wherein G is substituted two times with methyl.

6. The compound of claim 1, wherein R³ is independently selected from fluoro, hydroxyl, thiol, amido, NR⁴R⁵, C(O)NR⁴R⁵, C₁₋₄alkyl, C₁₋₄fluoroalkyl, C₁₋₄alkoxy and C₁₋₄alkylthio.

7. The compound of claim 1, wherein X is chloro and n is 0.

8. The compound of claim 1, wherein R¹ is any one selected from the group consisting of:
(i) C₁₋₄alkyl and C₁₋₄fluororalkyl; or
(ii) methyl or trifluoromethyl.

9. The compound of claim 1, wherein R² is H.

10. The compound of claim 1, wherein R is ethyl.

11. The degrader compound of claim 1, wherein D′ is any one selected from the group consisting of (CH₂)ₚC(O), (CH₂)_{q}NH, (CH₂)_{q}S, (CH₂)_{q}O, (CH₂)_{q} and 1,2,3-triazolylene, wherein p is an integer from 0 to 4 or is 0 and q is an integer from 1 to 4 or is 1.

12. The degrader compound of claim 1, wherein D′ is C(O).

13. The degrader compound of claim 1, wherein the degrader compound is of formula (IVA):

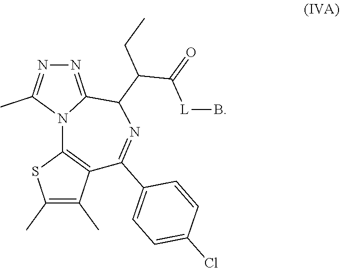

14. The degrader compound of claim 1, wherein L is of formula (VIA) 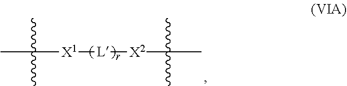
wherein
the wavy lines indicate the positions of attachment;
X¹ is optionally present and is any one selected from the group consisting of O(CH₂)ₛ, NH(CH₂)ₛ and C(O)(CH₂)ₛ;
X² is optionally present and is selected from the group consisting of O(CH₂)ᵤC(O), (CH₂)ᵤNH, (CH₂)ᵤO and (CH₂)ᵤC(O);
L′ is selected from the group consisting of O(CH₂)ₜ, CH₂, alkynylene, triazolylene, piperazinylene and piperidinylene;
s is an integer from 0 to 4, u is an integer from 1 to 4 and t is an integer from 1 to 4.

15. The degrader compound of claim 14, wherein X¹ is any one selected from the group consisting of O(CH₂)₂ and HN(CH₂)₂; X² is OCH₂C(O) or CH₂NH; and L′ is O(CH₂)ₜ, wherein t is 2 or 3.

16. The degrader compound of claim 1, wherein B is any one of the structures represented by any one of formulae (VIIA) to (XIA): 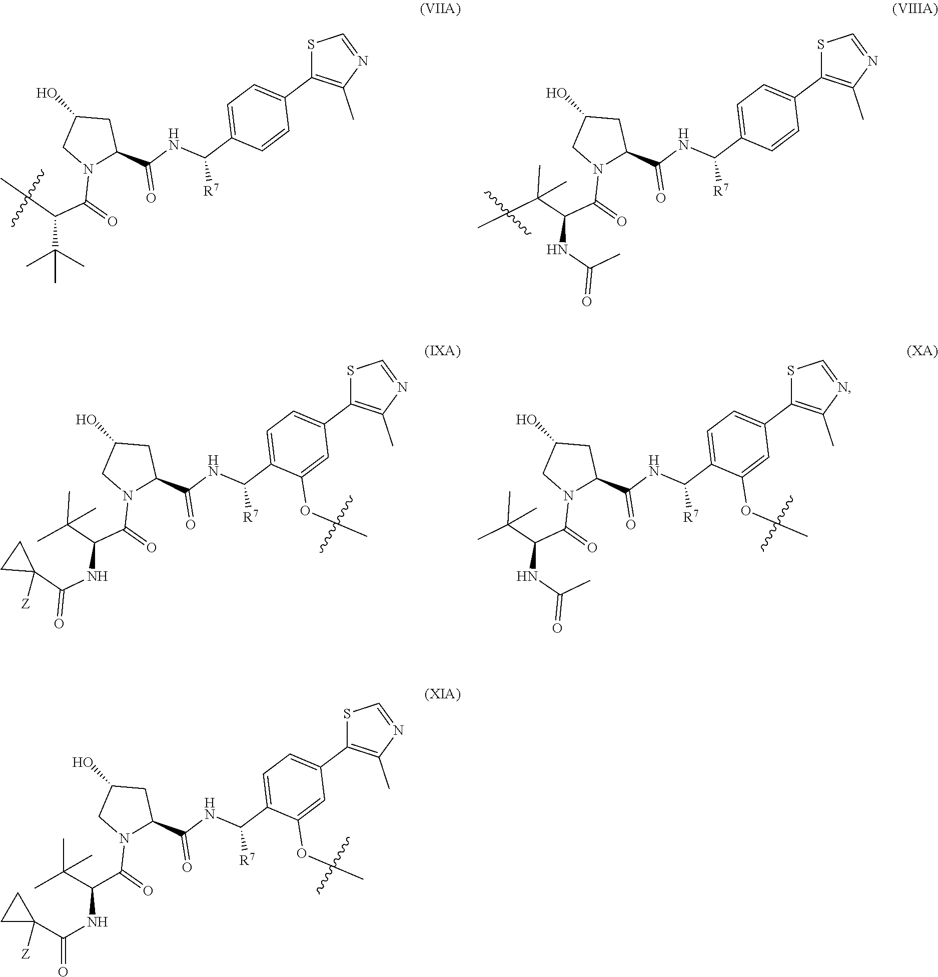
wherein R⁷ is H or methyl and Z is F or CN.

17. The degrader compound of claim 1, wherein the degrader compound is any one of formulae (XIXA) to (XXIA):

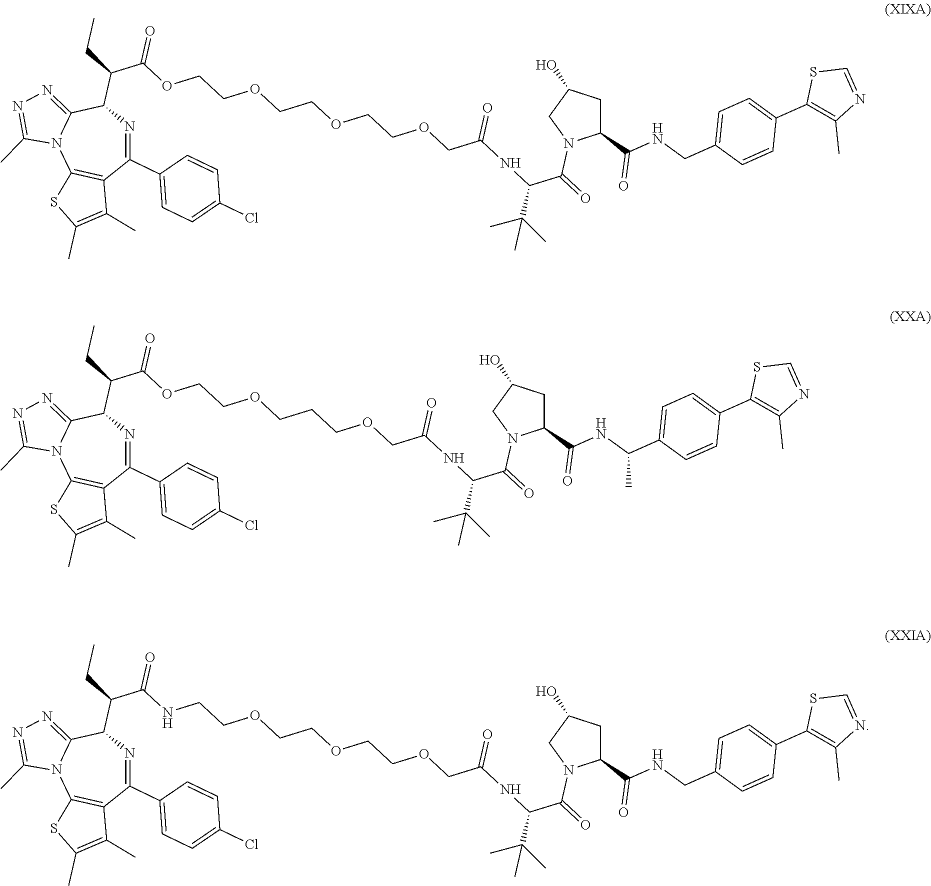 [SMILES: CC[C@@H](C(=O)OCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)OCCOCCCOCC(=O)N[C@H](C(=O)N1C[C@H](O)C[C@H]1C(=O)N[C@@H](C)c1ccc(-c2scnc2C)cc1)C(C)(C)C)[C@@H]1N=C(c2ccc(Cl)cc2)c2c(sc(C)c2C)-n2c(C)nnc21 ; CC[C@@H](C(=O)NCCOCCOCCOCC(=O)N[C@H](C(=O)N1C[C@H](O)C[C@H]1C(=O)NCc1ccc(-c2scnc2C)cc1)C(C)(C)C)[C@@H]1N=C(c2ccc(Cl)cc2)c2c(sc(C)c2C)-n2c(C)nnc21]

18.-20. (canceled)

21. A method of studying an effect of degrading a target protein within a cell, the method comprising:
endogenously expressing a fusion protein comprising the target protein, fused to a polypeptide comprising a hole-modified mutant bromodomain;
contacting the fusion protein with the degrader compound of claim 1; and
observing any effect upon the cell of degrading the target protein.

22. The method according to claim 21, wherein the target protein is endogenously tagged with one or more hole-modified mutant bromodomain(s) from the Bromo and Extraterminal domain (BET) proteins, Brd2, Brd3, Brd4 and BrdT, or a bromodomain containing fragment thereof.

23. The method according to claim 22, wherein the mutation occurs in one of more bromodomains which are present in the protein, or a bromodomain containing fragment thereof.

24. The method according to claim 22, wherein the hole-modified mutant bromodomain is a Brd4_{BD2}L387A, Brd4_{BD2}L387V, Brd4_{BD1}L94A, Brd4_{BD1}L94V, Brd2_{BD2}L383A, Brd2_{BD2}L383V, Brd2_{BD1}L110A, Brd2_{BD1}L110V, Brd3_{BD2}L344A, Brd3_{BD2}L344V, Brd3_{BD1}L70A, Brd3_{BD1}L70V, Brd4_{BD2}L306A, Brd4_{BD2}L306V, Brd4_{BD1}L63A, or Brd4_{BD1}L63V bromodomain.

25. The method according to claim 22, wherein the hole-modified mutant Brd4 bromodomain comprises a Brd4_{BD2}L387A or Brd4_{BD2}L387V bromodomain.
